(19) United States
(12) Patent Application Publication
(10) Pub. No.: US 2026/0049084 A1
(43) Pub. Date: Feb. 19, 2026

(54) ENPP1 MODULATORS AND PRODUCTS AND USES THEREOF

(71) Applicant: Gossamer Bio Services, Inc., San Diego, CA (US)

(72) Inventors: Martin William ROWBOTTOM, San Diego, CA (US); Kai LU, San Diego, CA (US); Theodore MICHELS, San Diego, CA (US); Michelle KASEM, San Diego, CA (US)

(21) Appl. No.: 18/865,105

(22) PCT Filed: May 12, 2023
(86) PCT No.: PCT/US2023/022091

§ 371 Date: Nov. 12, 2024

Related U.S. Application Data
(60) Provisional application No. 63/341,991, filed on May 13, 2022.

(51) Int. Cl. C07D 487/04; A61K 31/437; A61K 31/4545; A61K 31/519; C07D 471/04

(52) U.S. Cl. CPC C07D 487/04; A61K 31/437; A61K 31/4545; A61K 31/519; C07D 471/04

(57) ABSTRACT

Disclosed herein are compounds having the structure of Formula (I): or a pharmaceutically acceptable salt, solvate, hydrate, isomer, tautomer, racemate, or isotope thereof, wherein A, Q¹, R², R³, Q⁴, R⁵, R⁶, R⁷, R and n are as defined herein. Pharmaceutical compositions comprising them, processes for preparing them and uses of them to treat or prevent diseases, disorders and conditions are also provided. The compounds are inhibitors of ENPP1 and are useful in the treatment of diseases, disorders and conditions related to ENPP1 activity. In particular, the compounds are useful for treating a disease or condition selected from an infection (including but not limited to bacterial infections and viral infections), proliferative disorders (such as cancer or a tumor), cardiovascular disease (including cardiac injury), diabetes or insulin resistance, chondrocalcinosis, calcium pyrophosphate deposition disorder (pseudogout), or hypophosphatasia.

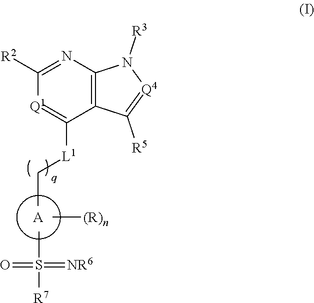

### FIELD OF THE INVENTION

The present invention generally relates to compounds that modulate ectonucleotide pyrophosphatase/phosphodiesterase 1 (ENPP1). Provided herein are compounds, compositions comprising such compounds, and methods of their use. The invention further pertains to pharmaceutical compositions comprising at least one compound according to the invention that are useful for the treatment of conditions, diseases, or disorders in which ENPP1 is involved, including infections (including but not limited to bacterial infections and viral infections), proliferative disorders (such as cancer or tumors), cardiovascular disease (including cardiac injury), diabetes or insulin resistance, chondrocalcinosis, calcium pyrophosphate deposition disorder (pseudogout), or hypophosphatasia.

### BACKGROUND

Cyclic GMP-AMP synthase (cGAS) belongs to the nucleotidyl transferase family and is a universal DNA sensor. It produces the signaling molecule cGAMP (or cyclic guanosine monophosphate-adenosine monophosphate or (2′-5′, 3′-5′) cyclic GMP-AMP or 2′, 3′-cGAMP or) from ATP and GTP in the presence of cytoplasmic DNA derived from microbial infection (viruses, bacteria), damaged mitochondria or cancer cells. cGAMP binds to and activates STING protein (the stimulator of interferon genes) to produce type I interferon (IFN), antiviral and immune-stimulatory cytokines, and other co-stimulatory molecules, that trigger the immune response. Thus, the cGAS enzyme, cGAMP messenger and STING are involved in host defense against infection and the immune control of tumor development.

The Ectonucleotide Pyrophosphatase/Phosphodiesterase (ENPP) family members include seven isoforms, ENPP1-7, which are type II transmembrane glycoproteins or ectoenzymes.

ENPP1 (Plasma cell membrane glycoprotein-1, PC-1) is present in a wide range of tissues and cell types, including lymphocytes, macrophages, liver, brain, heart, kidney, vascular smooth muscle cells, and chondrocytes. It has broad specificity and hydrolyses a variety of substrates including the phosphodiester & pyrophosphate bonds of nucleotides & nucleotide sugars.

ENPP1 naturally hydrolyzes cGAMP and therefore counteracts the innate immune response against infectious agents, damaged cells and cancer cells and has been implicated in a number of physiological processes and pathophysiological conditions.

ENPP1 expression is upregulated in certain hepatocellular carcinomas, glioblastomas, melanomas, testicular, pancreatic and thyroid and breast cancers and is associated with resistance to chemotherapy. Aberrant ENPP1 expression has been detected in lung and kidney fibrosis, breast cancers, bone metastasis, Hodgkin's lymphoma, hepatocellular carcinoma, follicular lymphoma, glioblastoma, and in other malignant tumor tissues.

Mutations in ENPP1 have been associated with insulin signaling and resistance and ENPP1 upregulation is linked to insulin resistance and type 2 diabetes.

Furthermore, ENPP1 activity has been implicated in diseases caused by bacteria and/or viruses; virus infection is facilitated by ENPP1 overexpression and is attenuated by ENPP1 silencing.

Inhibitors of ENPP1 may be useful for the treatment of inflammatory diseases associated with elevated nucleotidase levels, reduced nucleoside triphosphate, reduced cGAMP or reduced nucleoside monophosphate ester levels or diseases associated therewith.

Therefore, ENPP1 is an attractive target for the development of novel therapeutics, including anticancer, cardiovascular, diabetes, obesity and anti-fibrotic therapeutics or may be used to enhance the immune responses against cancer cells and tumors. Modulators of ENPP1 may be useful in treating bacterial and/or viral diseases and conditions, and also as vaccine boosters.

### BRIEF SUMMARY

In one embodiment, a compound is provided having the structure of Formula (I):

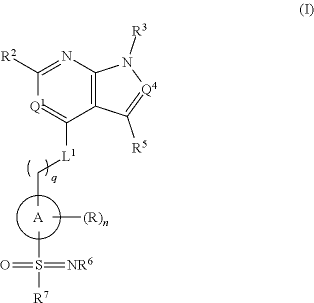

or a pharmaceutically acceptable salt, solvate, hydrate, isomer, tautomer, racemate, or isotope thereof, wherein
Q¹ is N or CR¹;
Q⁴ is N or CR⁴;
L¹ is —O— or —NR^{A}—
A is carbocycle or heterocycle;
R¹ is H, —CN, halo, C₁₋₄alkyl, C₁₋₄haloalkyl, C₂₋₄alkenyl, or C₂₋₄alkynyl;
R² is H, —CN, halo, C₁₋₄alkyl, C₁₋₄haloalkyl, carbocycle, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H, halo, or —CN;
R⁴ is H, —CN, halo, C₁₋₄alkyl, C₁₋₄haloalkyl, C₂₋₄alkenyl, C₂₋₄alkynyl, or C₃₋₅cycloalkyl;
R⁵ is H, —CN, halo, C₁₋₄alkyl, C₁₋₄haloalkyl, carbocycle, or heterocycle;
R⁶ is H, C₁₋₄alkyl, C₁₋₄haloalkyl, C₃₋₆cycloalkyl, —C₁₋₄alkyl-C₃₋₆cycloalkyl, or —CN;
R⁷ is —NR^{7a}R^{7b} or —C₁₋₄alkyl-R^{7c};
R^{7a} is H, C₁₋₄alkyl, or —CN;
R^{7b} is H, C₁₋₄alkyl, or —CN;
R^{7c} is H, —CN, halo, —NRR, —C(O)OR′, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or C₁₋₄haloalkyl;
R′ is H, C₁₋₄alkyl, or C₁₋₄haloalkyl;
R″ is H, C₁₋₄alkyl, or C₁₋₄haloalkyl;
n is 0, 1, 2, or 3; and
q is 0 or 1.

In some embodiments, a pharmaceutical composition is provided comprising a compound having the structure of Formula (I), or a pharmaceutically acceptable salt, solvate, hydrate, isomer, tautomer, racemate, or isotope thereof, and at least one pharmaceutically acceptable excipient.

In some embodiments, the compounds of Formula (I), or a pharmaceutically acceptable salt, solvate, hydrate, isomer, tautomer, racemate or isotope thereof, are useful as inhibitors of ENPP1.

In some embodiments, the compounds of Formula (I), or a pharmaceutically acceptable salt, solvate, hydrate, isomer, tautomer, racemate or isotope thereof, are useful in therapy. In some embodiments, the therapy is treatment of disorders including infections (including but not limited to bacterial infections and viral infections), proliferative disorders (such as cancer or tumors), cardiovascular disease (including cardiac injury), diabetes or insulin resistance, chondrocalcinosis, calcium pyrophosphate deposition disorder (pseudogout), or hypophosphatasia.

In some embodiments, a method of modulating the activity of ENPP1 is provided comprising contacting the kinase with an effective amount of a compound having the structure of Formula (I), or a pharmaceutically acceptable salt, solvate, hydrate, isomer, tautomer, racemate, isotope, or pharmaceutical composition thereof.

In some embodiments, a method for treating a disease or disorder associated with the activity of ENPP1 is provided, the method comprising administering to a subject in need thereof an effective amount of a compound having the structure of Formula (I), or a pharmaceutically acceptable salt, solvate, hydrate, isomer, tautomer, racemate, isotope, or pharmaceutical composition thereof.

In some embodiments, the use of a compound having the structure of Formula (I), or a pharmaceutically acceptable salt, solvate, hydrate, isomer, tautomer, racemate, isotope, or pharmaceutical composition thereof, is provided in the manufacture of a medicament. In some embodiments the medicament is useful for the treatment of diseases or disorders including infections (including but not limited to bacterial infections and viral infections), proliferative disorders (such as cancer or tumors), cardiovascular disease (including cardiac injury), diabetes or insulin resistance, chondrocalcinosis, calcium pyrophosphate deposition disorder (pseudogout), or hypophosphatasia.

In some embodiments, the compounds of Formula (I) and compositions comprising the compounds of Formula (I) may be used in treating, preventing, or curing diseases or disorders including infections (including but not limited to bacterial infections and viral infections), proliferative disorders (such as cancer or tumors), cardiovascular disease (including cardiac injury), diabetes or insulin resistance, chondrocalcinosis, calcium pyrophosphate deposition disorder (pseudogout), or hypophosphatasia. Pharmaceutical compositions comprising these compounds are useful in treating, preventing, or slowing the progression of diseases or disorders in a variety of therapeutic areas including infections (including but not limited to bacterial infections and viral infections), proliferative disorders (such as cancer or tumors), cardiovascular disease (including cardiac injury), diabetes or insulin resistance, chondrocalcinosis, calcium pyrophosphate deposition disorder (pseudogout), or hypophosphatasia.

In some embodiments, processes are provided for the preparation of the compounds of Formula (I) or a pharmaceutically acceptable salt, solvate, hydrate, isomer, tautomer, racemate, or isotope thereof.

In some embodiments, synthetic intermediates useful for the preparation of the compounds of Formula (I), or a pharmaceutically acceptable salt, solvate, hydrate, isomer, tautomer, racemate, or isotope thereof, are provided.

These and other features of the invention will be set forth in expanded form as the disclosure continues. The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

### DETAILED DESCRIPTION

As mentioned above, described herein are compounds that have activity as inhibitors of ectonucleotide pyrophosphatase/phosphodiesterase 1 (ENPP1). Further described herein are products comprising such compounds and their methods of use. In some embodiments, the compounds of the present invention that have activity as inhibitors of ENPP1 have selectivity over other enzymes, such as carbonic anhydrase.

Unless defined otherwise, all technical and scientific terms used herein have the same meaning as is commonly understood by one of skill in the art to which the claimed subject matter belongs. It is to be understood that the detailed description is exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe additional embodiments. It is also to be understood that each individual element of the embodiments is meant to be combined with any and all other elements from any embodiment to describe an additional embodiment.

As used herein, ranges and amounts can be expressed as “about” a particular value or range. About also includes the exact amount. Hence “about 100 μL” means “about 100 μL” and also “100 μL.” In some embodiments, about means within 5% of the value. Hence, “about 100 μL” means 95-105 μL. In some embodiments, about means within 4% of the value. In some embodiments, about means within 3% of the value. In some embodiments, about means within 2% of the value. In some embodiments, about means within 1% of the value. Generally, the term “about” includes an amount that would be expected to be within experimental error.

### Compounds

In one embodiment, a compound is provided having the structure of Formula (I):

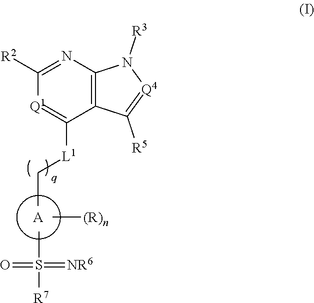

or a pharmaceutically acceptable salt, solvate, hydrate, isomer, tautomer, racemate, or isotope thereof, wherein
Q¹ is N or CR¹;
   Q⁴ is N or CR⁴;
   L¹ is —O— or —NR^{A}—
   A is carbocycle or heterocycle;
   R¹ is H, —CN, halo, C₁₋₄alkyl, C₁₋₄haloalkyl, C₂₋₄alkenyl, or C₂₋₄alkynyl;
   R² is H, —CN, halo, C₁₋₄alkyl, C₁₋₄haloalkyl, carbocycle, or heterocycle;
   R³ is H, —C₁₋₄alkyl-R^{3a}, C₁₋₄haloalkyl-R^{3a}, C₂₋₄alkenyl, or —C₂₋₄alkynyl;
   R^{3a} is H, halo, or —CN;
   R⁴ is H, —CN, halo, C₁₋₄alkyl, C₁₋₄haloalkyl, C₂₋₄alkenyl, C₂₋₄alkynyl, or C₃₋₅cycloalkyl;
   R⁵ is H, —CN, halo, C₁₋₄alkyl, C₁₋₄haloalkyl, carbocycle, or heterocycle;
   R⁶ is H, C₁₋₄alkyl, C₁₋₄haloalkyl, C₃₋₆cycloalkyl, —C₁₋₄alkyl-C₃₋₆cycloalkyl, or —CN;
   R⁷ is —NR^{7a}R^{7b} or —C₁₋₄alkyl-R^{7c};
   R^{7a} is H, C₁₋₄alkyl, or —CN;
   R^{7b} is H, C₁₋₄alkyl, or —CN;
   R^{7c} is H, —CN, halo, —NRR, —C(O)OR′, or —OR′;
   R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
   R^{A} is H, C₁₋₄alkyl, or C₁₋₄haloalkyl;
   R′ is H, C₁₋₄alkyl, or C₁₋₄haloalkyl;
   R″ is H, C₁₋₄alkyl, or C₁₋₄haloalkyl;
   n is 0, 1, 2, or 3; and
   q is 0 or 1.
   As used herein, “alkyl” means a straight chain or branched saturated hydrocarbon group. “Lower alkyl” means a straight chain or branched alkyl group having from 1 to 8 carbon atoms, in some embodiments from 1 to 6 carbon atoms, in some embodiments from 1 to 4 carbon atoms, and in some embodiments from 1 to 2 carbon atoms. Examples of straight chain lower alkyl groups include, but are not limited to, methyl, ethyl, n-propyl, n-butyl, n-pentyl, n-hexyl, n-heptyl, and n-octyl groups. Examples of branched lower alkyl groups include, but are not limited to, isopropyl, iso-butyl, sec-butyl, t-butyl, neopentyl, isopentyl, and 2,2-dimethylpropyl groups.

“Alkenyl” groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to —CH═CH₂, —CH═CH(CH₃), —CH═C(CH₃)₂, —C(CH₃)═CH₂, —C(CH₃)═CH(CH₃), —C(CH₂CH₃)═CH₂, —CH═CHCH₂CH₃, —CH═CH(CH₂)₂CH₃, —CH═CH(CH₂)₃CH₃, —CH═CH(CH₂)₄CH₃, vinyl, cyclohexenyl, cyclopentenyl, cyclohexadienyl, butadienyl, pentadienyl, and hexadienyl among others.

“Alkynyl” groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃), among others.

As used herein, “alkylene” means a divalent alkyl group. Examples of straight chain lower alkylene groups include, but are not limited to, methylene (i.e., —CH₂—), ethylene (i.e., —CH₂CH₂—), propylene (i.e., —CH₂CH₂CH₂—), and butylene (i.e., —CH₂CH₂CH₂CH₂—). As used herein, “heteroalkylene” is an alkylene group of which one or more carbon atoms is replaced with a heteroatom such as, but not limited to, N, O, S, or P.

“Alkoxy” refers to an alkyl as defined above joined by way of an oxygen atom (i.e., —O-alkyl). Examples of lower alkoxy groups include, but are not limited to, methoxy, ethoxy, n-propoxy, n-butoxy, isopropoxy, sec-butoxy, tert-butoxy, and the like.

The terms “carbocyclic” and “carbocycle” denote a ring structure wherein the atoms of the ring are carbon. Carbocycles may be monocyclic or polycyclic. Carbocycle encompasses both saturated and unsaturated rings. Carbocycle encompasses both cycloalkyl and aryl groups. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N substituents wherein N is the size of the carbocyclic ring with for example, alkyl, amino, hydroxy, cyano, carboxy, nitro, thio, alkoxy, and halogen groups.

“Cycloalkyl” groups are alkyl groups forming a ring structure, which can be substituted or unsubstituted. Examples of cycloalkyl include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Unless specifically indicated to the contrary, the cycloalkyl ring can be substituted. Representative substituted cycloalkyl groups can be mono-substituted, such as, but not limited to, 1-, 2-, 3-, or 4-substituted cyclobutyl, or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, straight or branched chain alkyl groups as defined above, amino, hydroxy, cyano, carboxy, nitro, thio, alkoxy, and halogen groups.

“Aryl” groups are cyclic aromatic hydrocarbons that do not contain heteroatoms. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The terms “aryl” and “aryl groups” include fused rings wherein at least one ring, but not necessarily all rings, are aromatic, such as fused aromatic-aliphatic ring systems (e.g., indanyl, tetrahydronaphthyl, and the like). Unless specifically indicated to the contrary, aryl can be substituted.

“Carbocyclealkyl” refers to an alkyl as defined above with one or more hydrogen atoms replaced with carbocycle. Examples of carbocyclealkyl groups include, but are not limited to, benzyl and the like. Unless specifically indicated to the contrary, carbocyclealkyl can be substituted.

As used herein, “heterocycle” or “heterocyclyl” groups include aromatic and non-aromatic ring compounds (heterocyclic rings) containing 3 or more ring members, of which one or more is a heteroatom such as, but not limited to, N, O, S, or P. A heterocycle group as defined herein can be a heteroaryl group or a partially or completely saturated cyclic group including at least one ring heteroatom. In some embodiments, heterocycle groups include 3 to 20 ring members, whereas other such groups have 3 to 15 ring members. At least one ring contains a heteroatom, but every ring in a polycyclic system need not contain a heteroatom. For example, a dioxolanyl ring and a benzodioxolanyl ring system (methylenedioxyphenyl ring system) are both heterocycle groups within the meaning herein. A heterocycle group designated as a C₂-heterocycle can be a 5-membered ring with two carbon atoms and three heteroatoms, a 6-membered ring with two carbon atoms and four heteroatoms and so forth. Likewise, a C₄-heterocycle can be a 5-membered ring with one heteroatom, a 6-membered ring with two heteroatoms, and so forth. The number of carbon atoms plus the number of heteroatoms sums up to equal the total number of ring atoms. A saturated heterocyclic ring refers to a heterocyclic ring containing no unsaturated carbon atoms. Unless specifically indicated to the contrary, the heterocycle can be substituted with as many as N substituents wherein N is the size of the heterocyclic ring with for example, alkyl, amino, hydroxy, cyano, carboxy, nitro, thio, alkoxy, and halogen groups. Representative substituted heterocycle groups can be mono-substituted, such as, but not limited to, 2-, or 3-substituted oxetan-3-yl or 2-, 3-, or 4-substituted tetrahydropyran-4-yl.

“Heteroaryl” groups are aromatic ring compounds containing 5 or more ring members, of which, one or more is a heteroatom such as, but not limited to, N, O, and S. A heteroaryl group designated as a C₂-heteroaryl can be a 5-membered ring with two carbon atoms and three heteroatoms, a 6-membered ring with two carbon atoms and four heteroatoms and so forth. Likewise, a C₄-heteroaryl can be a 5-membered ring with one heteroatom, a 6-membered ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl and 2,3-dihydro indolyl. Unless specifically indicated to the contrary, the heteroaryl can be substituted with as many as N substituents wherein N is the size of the heteroaryl ring.

“Heterocyclealkyl” refers to an alkyl as defined above with one or more hydrogen atoms replaced with heterocycle. Examples of heterocyclealkyl groups include, but are not limited to, morpholinoethyl and the like. Unless specifically indicated to the contrary, heterocyclealkyl can be substituted.

“Halo” or “halogen” refers to fluorine, chlorine, bromine, and iodine.

“Haloalkyl” refers to an alkyl as defined above with one or more hydrogen atoms replaced with halogen. Examples of lower haloalkyl groups include, but are not limited to, —CF₃, —CH₂CF₃, and the like.

“Haloalkoxy” refers to an alkoxy as defined above with one or more hydrogen atoms replaced with halogen. Examples of lower haloalkoxy groups include, but are not limited to —OCF₃, —OCH₂CF₃, and the like.

“Hydroxyalkyl” refers to an alkyl as defined above with one or more hydrogen atoms replaced with —OH. Examples of lower hydroxyalkyl groups include, but are not limited to —CH₂OH, —CH₂CH₂OH, and the like.

As used herein, the term “substituted” or “optionally substituted” refers to a group (e.g., an alkyl, carbocycle, or heterocycle) having 0, 1, or more substituents, such as 0-25, 0-20, 0-10 or 0-5 substituents. Substituents include, but are not limited to —OR^{a}, —NR^{a}R^{b}, —S(O)₂R^{a}, —S(O)₂OR^{a}, halogen, cyano, alkyl, haloalkyl, alkoxy, carbocycle, heterocycle, carbocyclealkyl, or heterocyclealkyl, wherein each R^{a} and R^{b} is, independently, H, alkyl, haloalkyl, carbocycle, or heterocycle, or R^{a} and R^{b}, together with the atom to which they are attached, form a 3-8 membered carbocycle or heterocycle.

In one embodiment, a compound of Formula (I) is provided, or a pharmaceutically acceptable salt, solvate, hydrate, isomer, tautomer, racemate, or isotope thereof, wherein A is a carbocycle.

In one embodiment, a compound of Formula (I) is provided, or a pharmaceutically acceptable salt, solvate, hydrate, isomer, tautomer, racemate, or isotope thereof, wherein A is cycloalkyl. In one embodiment, A is cyclopentyl, cyclohexyl, or cycloheptyl.

In one embodiment, a compound of Formula (I) is provided, or a pharmaceutically acceptable salt, solvate, hydrate, isomer, tautomer, racemate, or isotope thereof, wherein A is aryl.

In one embodiment, a compound of Formula (I) is provided, or a pharmaceutically acceptable salt, solvate, hydrate, isomer, tautomer, racemate, or isotope thereof, wherein Q¹ is N. In another embodiment, Q¹ is CR¹.

In one embodiment, a compound of Formula (I) is provided, or a pharmaceutically acceptable salt, solvate, hydrate, isomer, tautomer, racemate, or isotope thereof, wherein Q⁴ is N. In another embodiment, Q⁴ is CR⁴.

In one embodiment, a compound having the structure of Formula (II) is provided:

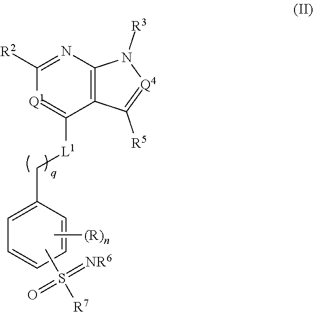

or a pharmaceutically acceptable salt, solvate, hydrate, isomer, tautomer, racemate, or isotope thereof, wherein
Q¹ is N or CR¹;
Q⁴ is N or CR⁴;
L¹ is —O— or —NR^{A}—;
R¹ is H, —CN, halo, C₁₋₄alkyl, C₁₋₄haloalkyl, C₂₋₄alkenyl, or C₂₋₄alkynyl;
R² is H, —CN, halo, C₁₋₄alkyl, C₁₋₄haloalkyl, carbocycle, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H, halo, or —CN;
R⁴ is H, —CN, halo, C₁₋₄alkyl, C₁₋₄haloalkyl, C₂₋₄alkenyl, C₂₋₄alkynyl, or C₃₋₅cycloalkyl;
R⁵ is H, —CN, halo, C₁₋₄alkyl, C₁₋₄haloalkyl, carbocycle, or heterocycle;
R⁶ is H, C₁₋₄alkyl, C₁₋₄haloalkyl, C₃₋₆cycloalkyl, —C₁₋₄alkyl-C₃₋₆cycloalkyl, or —CN;
R⁷ is —NR^{7a}R^{7b} or —C₁₋₄alkyl-R^{7c};
R^{7a} is H, C₁₋₄alkyl, or —CN;
R^{7b} is H, C₁₋₄alkyl, or —CN;
R^{7c} is H, —CN, halo, —NRR′, —C(O)OR′, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or haloalkyl;
R′ is H, C₁₋₄alkyl, or C₁₋₄haloalkyl;
R″ is H, C₁₋₄alkyl, or C₁₋₄haloalkyl;
n is 0, 1, 2, or 3; and
q is 0 or 1.

In one embodiment, a compound of Formula (II) is provided, or a pharmaceutically acceptable salt, solvate, hydrate, isomer, tautomer, racemate, or isotope thereof, wherein Q¹ is N. In another embodiment, Q¹ is CR¹.

In one embodiment, a compound of Formula (II) is provided, or a pharmaceutically acceptable salt, solvate, hydrate, isomer, tautomer, racemate, or isotope thereof, wherein Q⁴ is N. In another embodiment, Q⁴ is CR⁴.

In one embodiment, a compound is provided having the structure of any one of Formula (III), Formula (IV), Formula (V), or Formula (VI):

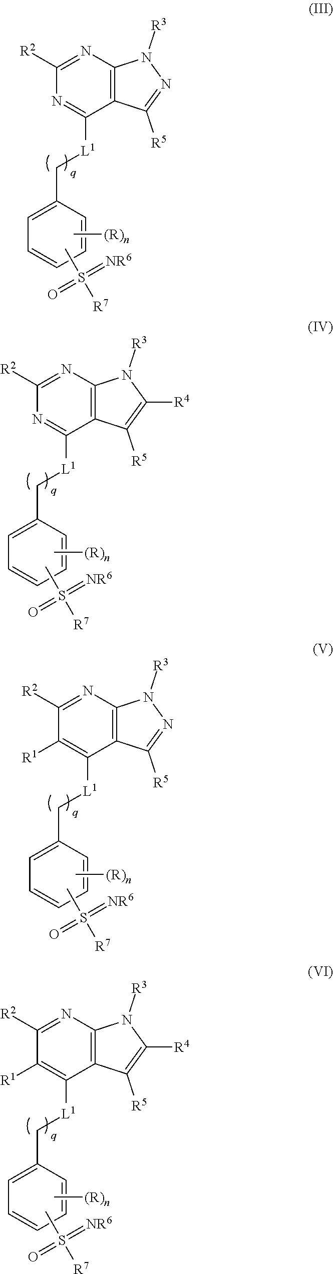

or a pharmaceutically acceptable salt, solvate, hydrate, isomer, tautomer, racemate, or isotope thereof, wherein
L¹ is —O— or —NR^{A}—;
R¹ is H, —CN, halo, C₁₋₄alkyl, C₁₋₄haloalkyl, C₂₋₄alkenyl, or C₂₋₄alkynyl;
R² is H, —CN, halo, C₁₋₄alkyl, C₁₋₄haloalkyl, carbocycle, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H, halo, or —CN;
R⁴ is H, —CN, halo, C₁₋₄alkyl, C₁₋₄haloalkyl, C₂₋₄alkenyl, C₂₋₄alkynyl, or C₃₋₅cycloalkyl;
R⁵ is H, —CN, halo, C₁₋₄alkyl, C₁₋₄haloalkyl, carbocycle, or heterocycle;
R⁶ is H, C₁₋₄alkyl, C₁₋₄haloalkyl, C₃₋₆cycloalkyl, —C₁₋₄alkyl-C₃₋₆cycloalkyl, or —CN;
R⁷ is —NR^{7a}R^{7b} or —C₁₋₄alkyl-R^{7c};
R^{7a} is H, C₁₋₄alkyl, or —CN;
R^{7b} is H, C₁₋₄alkyl, or —CN;
R^{7c} is H, —CN, halo, —NRR, —C(O)OR′, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or haloalkyl;
R′ is H, C₁₋₄alkyl, or C₁₋₄haloalkyl;
R″ is H, C₁₋₄alkyl, or C₁₋₄haloalkyl;
n is 0, 1, 2, or 3; and
q is 0 or 1.

In one embodiment, a compound of any one of Formula (I), Formula (II), Formula (III), Formula (IV), Formula (V), or Formula (VI) is provided, or a pharmaceutically acceptable salt, solvate, hydrate, isomer, tautomer, racemate, or isotope thereof, wherein q is 0. In another embodiment, q is 1.

In one embodiment, a compound of Formula (III) is provided having the structure of any one of Formula (III-A), Formula (III-B), or Formula (III-C):

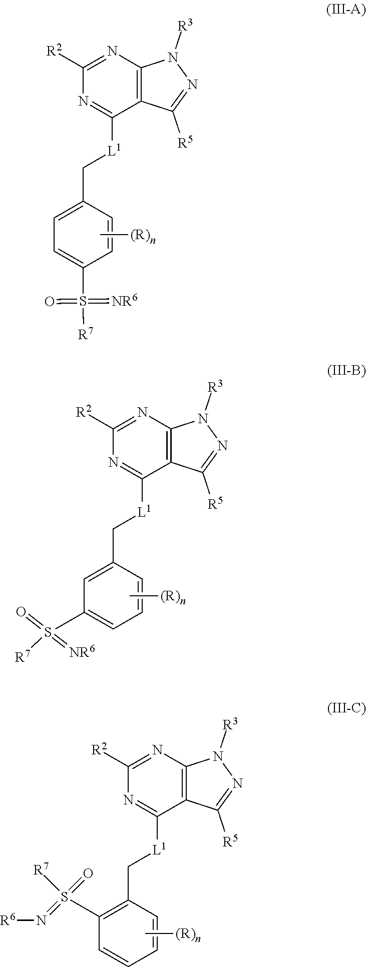

or a pharmaceutically acceptable salt, solvate, hydrate, isomer, tautomer, racemate, or isotope thereof, wherein
L¹ is —O— or —NR^{A}—;
R² is H, —CN, halo, C₁₋₄alkyl, C₁₋₄haloalkyl, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H or —CN;
R⁵ is H, halo, C₁₋₄alkyl, C₁₋₄haloalkyl, carbocycle, or heterocycle;
R⁶ is H, C₁₋₄alkyl, or —CN;
R⁷ is —NR^{7a}R^{7b} or —C₁₋₄alkyl-R^{7c};
R^{7a} is H or C₁₋₄alkyl;
R^{7b} is H or C₁₋₄alkyl;
R^{7c} is H, —CN, halo, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or haloalkyl;
R′ is H, C₁₋₄alkyl, or haloalkyl;
R″ is H, C₁₋₄alkyl, or haloalkyl; and
n is 0, 1, 2, or 3.

In one embodiment, a compound of Formula (IV) is provided having the structure of any one of Formula (IV-A), Formula (IV-B), or Formula (IV-C):

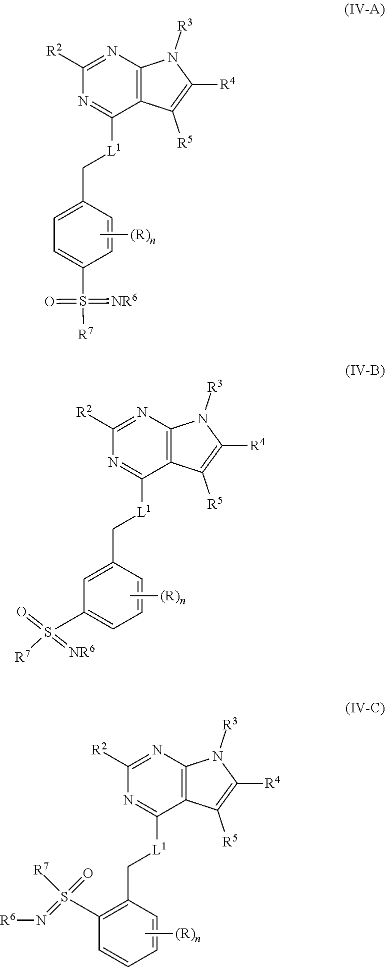

or a pharmaceutically acceptable salt, solvate, hydrate, isomer, tautomer, racemate, or isotope thereof, wherein
L¹ is —O— or —NR^{A}—;
R² is H, —CN, halo, C₁₋₄alkyl, C₁₋₄haloalkyl, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H or —CN;
R⁴ is H, —CN, halo, C₁₋₄alkyl, C₁₋₄haloalkyl, C₂₋₄alkenyl, C₂₋₄alkynyl, or C₃₋₅cycloalkyl;
R⁵ is H, halo, C₁₋₄alkyl, C₁₋₄haloalkyl, carbocycle, or heterocycle;
R⁶ is H, C₁₋₄alkyl, or —CN;
R⁷ is —NR^{7a}R^{7b} or —C₁₋₄alkyl-R^{7c};
R^{7a} is H or C₁₋₄alkyl;
R^{7b} is H or C₁₋₄alkyl;
R^{7c} is H, —CN, halo, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or haloalkyl;
R′ is H, C₁₋₄alkyl, or haloalkyl;
R″ is H, C₁₋₄alkyl, or haloalkyl; and
n is 0, 1, 2, or 3.

In one embodiment, a compound of Formula (V) is provided having the structure of any one of Formula (V-A), Formula (V-B), or Formula (V-C):

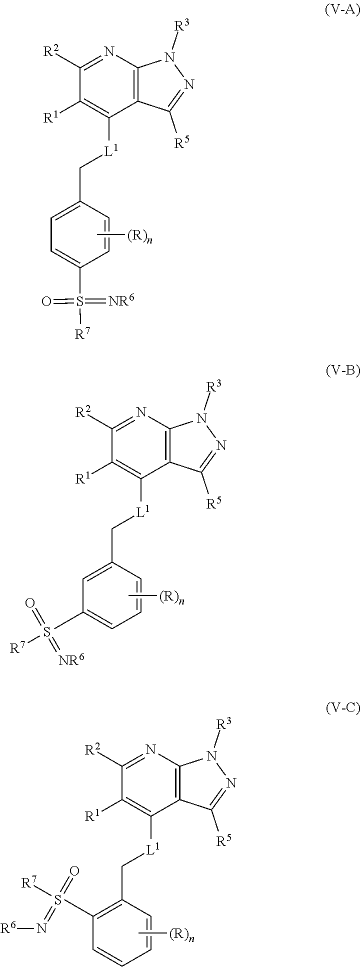

or a pharmaceutically acceptable salt, solvate, hydrate, isomer, tautomer, racemate, or isotope thereof, wherein
L¹ is —O— or —NR^{A}—;
R¹ is H, —CN, halo, C₁₋₄alkyl, C₁₋₄haloalkyl, C₂₋₄alkenyl, or C₂₋₄alkynyl;
R² is H, —CN, halo, C₁₋₄alkyl, C₁₋₄haloalkyl, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H or —CN;
R⁵ is H, halo, C₁₋₄alkyl, C₁₋₄haloalkyl, carbocycle, or heterocycle;
R⁶ is H, C₁₋₄alkyl, or —CN;
R⁷ is —NR^{7a}R^{7b} or —C₁₋₄alkyl-R^{7c};
R^{7a} is H or C₁₋₄alkyl;
R^{7b} is H or C₁₋₄alkyl;
R^{7c} is H, —CN, halo, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or haloalkyl;
R′ is H, C₁₋₄alkyl, or haloalkyl;
R″ is H, C₁₋₄alkyl, or haloalkyl; and
n is 0, 1, 2, or 3.

In one embodiment, a compound of Formula (VI) is provided having the structure of any one of Formula (VI-A), Formula (VI-B), or Formula (VI-C):

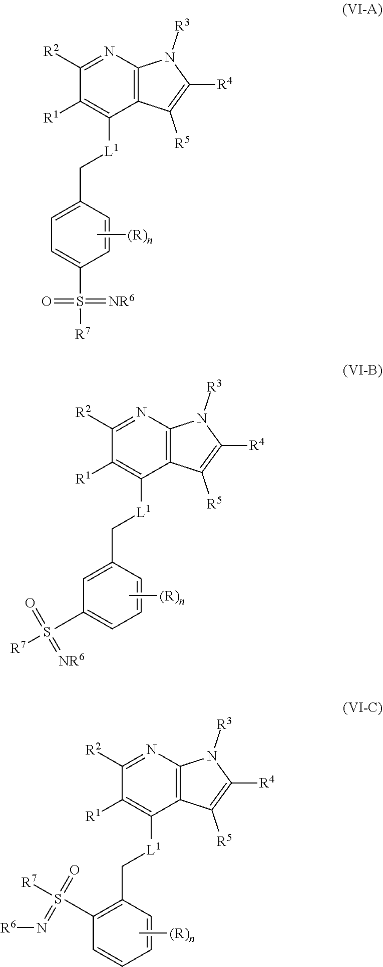

or a pharmaceutically acceptable salt, solvate, hydrate, isomer, tautomer, racemate, or isotope thereof, wherein
L¹ is —O— or —NR^{A}—;
R¹ is H, —CN, halo, C₁₋₄alkyl, C₁₋₄haloalkyl, C₂₋₄alkenyl, or C₂₋₄alkynyl;
R² is H, —CN, halo, C₁₋₄alkyl, C₁₋₄haloalkyl, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H or —CN;
R⁴ is H, —CN, halo, C₁₋₄alkyl, C₁₋₄haloalkyl, C₂₋₄alkenyl, C₂₋₄alkynyl, or C₃₋₅cycloalkyl;
R⁵ is H, halo, C₁₋₄alkyl, C₁₋₄haloalkyl, carbocycle, or heterocycle;
R⁶ is H, C₁₋₄alkyl, or —CN;
R⁷ is —NR^{7a}R^{7b} or —C₁₋₄alkyl-R^{7c};
R^{7a} is H or C₁₋₄alkyl;
R^{7b} is H or C₁₋₄alkyl;
R^{7c} is H, —CN, halo, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or haloalkyl;
R′ is H, C₁₋₄alkyl, or haloalkyl;
R″ is H, C₁₋₄alkyl, or haloalkyl; and
n is 0, 1, 2, or 3.

In one embodiment, a compound of Formula (I) is provided, or a pharmaceutically acceptable salt, solvate, hydrate, isomer, tautomer, racemate, or isotope thereof, wherein A is a heterocycle.

In one embodiment, a compound of Formula (I) is provided, or a pharmaceutically acceptable salt, solvate, hydrate, isomer, tautomer, racemate, or isotope thereof, wherein A is a non-aromatic heterocycle. In one embodiment, A is tetrahydrofuranyl, pyrrolidinyl, thiolanyl, azetidinyl, oxetanyl, thietanyl, piperidinyl, piperazinyl, 2H-pyranyl, dihydro-2H-pyranyl, tetrahydro-2H-pyranyl, 1,4-dioxane, morpholino, azepanyl, diazepanyl, oxepanyl, or thiepanyl. In another embodiment, A is pyrrolidinyl, tetrahydrofuranyl, piperidinyl, or piperazinyl.

In one embodiment, a compound of Formula (I) is provided, or a pharmaceutically acceptable salt, solvate, hydrate, isomer, tautomer, racemate, or isotope thereof, wherein A is heteroaryl. In one embodiment, A is thiophenyl, thiazolyl, isthiazolyl, thiadiazolyl, furanyl, oxazolyl, isoxazolyl, oxadiazolyl, pyrrolyl, imidazolyl, pyrazolyl, triazolyl, pyridinyl, pyrazinyl, pyrimidinyl, triazinyl, indolyl, benzimidazolyl, indazolyl, benzotriazolyl, pyrrolopyridinyl, pyrrolopyrimidinyl, purinyl, indolizinyl, pyrrolopyrazinyl, pyrrolopyriminyl, pyrrolopyridazinyl, imidazopyridinyl, pyrazolopyridinyl, imidazopyridinyl, imidazopyridazinyl, imidazopyrimidinyl, imidazopyrazinyl, triazolopyridinyl, quinolinyl, isoquinolinyl, cinnolinyl, quinazolinyl, quinoxalinyl, phthalazinyl, pyridopyrazinyl, pteridinyl, pyridopyridazinyl, naphthyridinyl, carbazolyl, benzoxazolyl, dibenzofuranyl, benzothiophenyl, or dibenzothiphenyl.

In one embodiment, a compound is provided having the structure of Formula (VII):

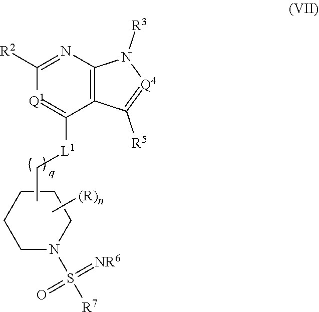

or a pharmaceutically acceptable salt, solvate, hydrate, isomer, tautomer, racemate, or isotope thereof, wherein
L¹ is —O— or —NR^{A}—;
Q¹ is N or CR¹;
Q⁴ is N or CR⁴;
R¹ is H, —CN, halo, C₁₋₄alkyl, C₁₋₄haloalkyl, C₂₋₄alkenyl, or C₂₋₄alkynyl;
R² is H, —CN, halo, C₁₋₄alkyl, C₁₋₄haloalkyl, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H or —CN;
R⁴ is H, —CN, halo, C₁₋₄alkyl, C₁₋₄haloalkyl, C₂₋₄alkenyl, C₂₋₄alkynyl, or C₃₋₅cycloalkyl;
R⁵ is H, halo, C₁₋₄alkyl, C₁₋₄haloalkyl, carbocycle, or heterocycle;
R⁶ is H, C₁₋₄alkyl, or —CN;
R⁷ is —NR^{7a}R^{7b} or —C₁₋₄alkyl-R^{7c};
R^{7a} is H or C₁₋₄alkyl;
R^{7b} is H or C₁₋₄alkyl;
R^{7c} is H, —CN, halo, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or haloalkyl;
R′ is H, C₁₋₄alkyl, or haloalkyl;
R″ is H, C₁₋₄alkyl, or haloalkyl;
n is 0, 1, 2, or 3; and
q is 0 or 1.

In one embodiment, a compound having the structure of Formula (VII) is provided, or a pharmaceutically acceptable salt, solvate, hydrate, isomer, tautomer, racemate, or isotope thereof, wherein Q¹ is N. In another embodiment, Q¹ is CR¹.

In one embodiment, a compound having the structure of Formula (VII) is provided, or a pharmaceutically acceptable salt, solvate, hydrate, isomer, tautomer, racemate, or isotope thereof, wherein Q⁴ is N. In another embodiment, Q⁴ is CR⁴.

In one embodiment, a compound of Formula (VII) is provided having the structure of any one of Formula (VIII), Formula (IX), Formula (X), or Formula (XI):

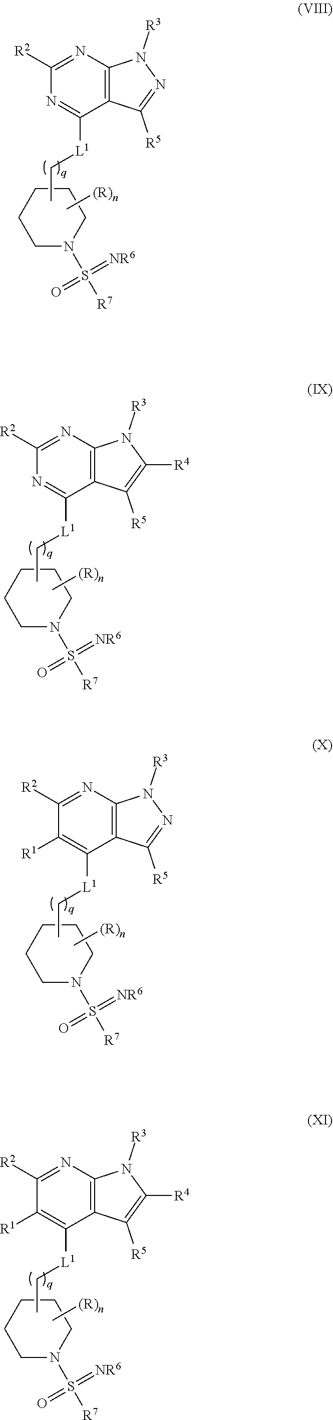

or a pharmaceutically acceptable salt, solvate, hydrate, isomer, tautomer, racemate, or isotope thereof, wherein
L¹ is —O— or —NR^{A}—;
R¹ is H, —CN, halo, C₁₋₄alkyl, C₁₋₄haloalkyl, C₂₋₄alkenyl, or C₂₋₄alkynyl;
R² is H, —CN, halo, C₁₋₄alkyl, C₁₋₄haloalkyl, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H or —CN;
R⁴ is H, —CN, halo, C₁₋₄alkyl, C₁₋₄haloalkyl, C₂₋₄alkenyl, C₂₋₄alkynyl, or C₃₋₅cycloalkyl;
R⁵ is H, halo, C₁₋₄alkyl, C₁₋₄haloalkyl, carbocycle, or heterocycle;
R⁶ is H, C₁₋₄alkyl, or —CN;
R⁷ is —NR^{7a}R^{7b} or —C₁₋₄alkyl-R^{7c};
R^{7a} is H or C₁₋₄alkyl;
R^{7b} is H or C₁₋₄alkyl;
R^{7c} is H, —CN, halo, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or haloalkyl;
R′ is H, C₁₋₄alkyl, or haloalkyl;
R″ is H, C₁₋₄alkyl, or haloalkyl;
n is 0, 1, 2, or 3; and
q is 0 or 1.

In one embodiment, a compound of Formula (VIII) is provided having the structure of any one of Formula (VIII-A), Formula (VIII-B), or Formula (VIII-C),

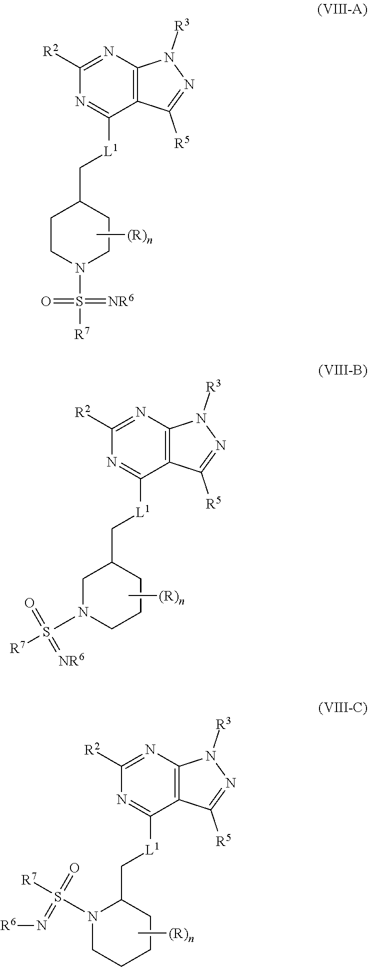

or a pharmaceutically acceptable salt, solvate, hydrate, isomer, tautomer, racemate, or isotope thereof, wherein
L¹ is —O— or —NR^{A}—;
R² is H, —CN, halo, C₁₋₄alkyl, C₁₋₄haloalkyl, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H or —CN;
R⁵ is H, halo, C₁₋₄alkyl, C₁₋₄haloalkyl, carbocycle, or heterocycle;
R⁶ is H, C₁₋₄alkyl, or —CN;
R⁷ is —NR^{7a}R^{7b} or —C₁₋₄alkyl-R^{7c};
R^{7a} is H or C₁₋₄alkyl;
R^{7b} is H or C₁₋₄alkyl;
R^{7c} is H, —CN, halo, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or haloalkyl;
R′ is H, C₁₋₄alkyl, or haloalkyl;
R″ is H, C₁₋₄alkyl, or haloalkyl; and
n is 0, 1, 2, or 3.

In one embodiment, a compound of Formula (IX) is provided having the structure of any one of Formula (IX-A), Formula (IX-B), or Formula (IX-C),

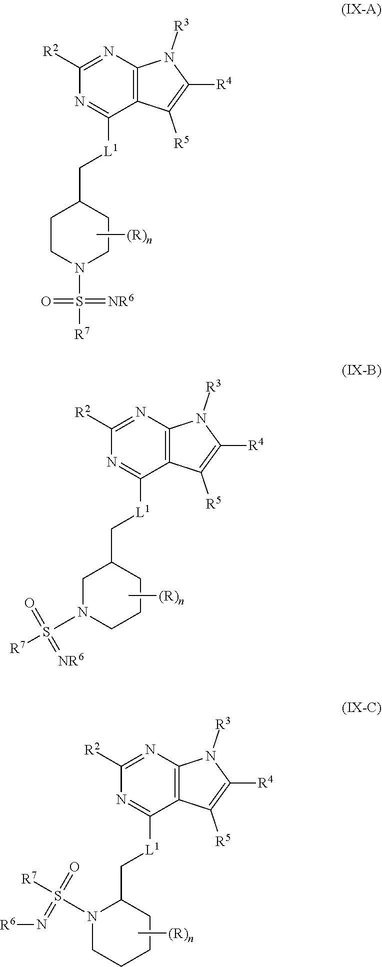

or a pharmaceutically acceptable salt, solvate, hydrate, isomer, tautomer, racemate, or isotope thereof, wherein
L¹ is —O— or —NR^{A}—;
R² is H, —CN, halo, C₁₋₄alkyl, C₁₋₄haloalkyl, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H or —CN;
R⁴ is H, —CN, halo, C₁₋₄alkyl, C₁₋₄haloalkyl, C₂₋₄alkenyl, C₂₋₄alkynyl, or C₃₋₅cycloalkyl;
R⁵ is H, halo, C₁₋₄alkyl, C₁₋₄haloalkyl, carbocycle, or heterocycle;
R⁶ is H, C₁₋₄alkyl, or —CN;
R⁷ is —NR^{7a}R^{7b} or —C₁₋₄alkyl-R^{7c};
R^{7a} is H or C₁₋₄alkyl;
R^{7b} is H or C₁₋₄alkyl;
R^{7c} is H, —CN, halo, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or haloalkyl;
R′ is H, C₁₋₄alkyl, or haloalkyl;
R″ is H, C₁₋₄alkyl, or haloalkyl; and
n is 0, 1, 2, or 3.

In one embodiment, a compound of Formula (X) is provided having the structure of any one of Formula (X-A), Formula (X-B), or Formula (X-C),

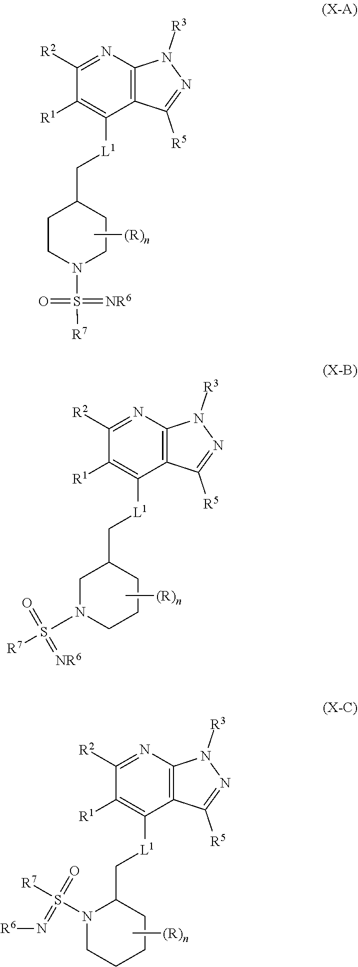

or a pharmaceutically acceptable salt, solvate, hydrate, isomer, tautomer, racemate, or isotope thereof, wherein
L¹ is —O— or —NR^{A}—;
R¹ is H, —CN, halo, C₁₋₄alkyl, C₁₋₄haloalkyl, C₂₋₄alkenyl, or C₂₋₄alkynyl;
R² is H, —CN, halo, C₁₋₄alkyl, C₁₋₄haloalkyl, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H or —CN;
R⁵ is H, halo, C₁₋₄alkyl, C₁₋₄haloalkyl, carbocycle, or heterocycle;
R⁶ is H, C₁₋₄alkyl, or —CN;
R⁷ is —NR^{7a}R^{7b} or —C₁₋₄alkyl-R^{7c};
R^{7a} is H or C₁₋₄alkyl;
R^{7b} is H or C₁₋₄alkyl;
R^{7c} is H, —CN, halo, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or haloalkyl;
R′ is H, C₁₋₄alkyl, or haloalkyl;
R″ is H, C₁₋₄alkyl, or haloalkyl; and
n is 0, 1, 2, or 3.

In one embodiment, a compound of Formula (XI) is provided having the structure of any one of Formula (XI-A), Formula (XI-B), or Formula (XI-C),

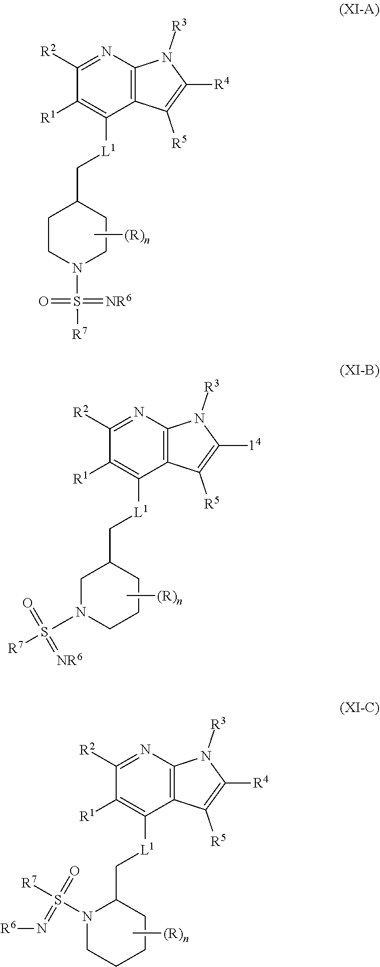

or a pharmaceutically acceptable salt, solvate, hydrate, isomer, tautomer, racemate, or isotope thereof, wherein
L¹ is —O— or —NR^{A}—
R¹ is H, —CN, halo, C₁₋₄alkyl, C₁₋₄haloalkyl, C₂₋₄alkenyl, or C₂₋₄alkynyl;
R² is H, —CN, halo, C₁₋₄alkyl, C₁₋₄haloalkyl, or heterocycle;
R³ is H, —C₁₋₄alkyl-R^{3a}, C₁₋₄haloalkyl-R^{3a}, C₂₋₄alkenyl, or —C₂₋₄alkynyl;
R^{3a} is H or —CN;
R⁴ is H, —CN, halo, C₁₋₄alkyl, C₁₋₄haloalkyl, C₂₋₄alkenyl, C₂₋₄alkynyl, or C₃₋₅cycloalkyl;
R⁵ is H, halo, C₁₋₄alkyl, C₁₋₄haloalkyl, carbocycle, or heterocycle;
R⁶ is H, C₁₋₄alkyl, or —CN;
R⁷ is —NR^{7a}R^{7b} or —C₁₋₄alkyl-R^{7c};
R^{7a} is H or C₁₋₄alkyl;
R^{7b} is H or C₁₋₄alkyl;
R^{7c} is H, —CN, halo, or —OR′;
R is, at each occurrence, independently H, —CN, halo, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, —OC(O)-heterocycle, —NR′R″, C₁₋₄alkyl-NR′R″, —NR′—C₁₋₄alkyl, —C₁₋₄haloalkyl-NR′R″, —NR′—C₁₋₄haloalkyl, —NR′-carbocycle, —NR′-heterocycle, —C(O)NR′R″, C₁₋₄alkyl-C(O)NR′R″, —C(O)NR′—C₁₋₄alkyl, —C₁₋₄haloalkyl-C(O)NR′R″, —C(O)NR′—C₁₋₄haloalkyl, —C(O)NR′-carbocycle, —C(O)NR′-heterocycle, —NR′C(O)R″, C₁₋₄alkyl-NR′C(O)R″, —NR′C(O)—C₁₋₄alkyl, —C₁₋₄haloalkyl-NR′C(O)R″, —NR′C(O)—C₁₋₄haloalkyl, —NR′C(O)-carbocycle, or —NR′C(O)-heterocycle;
R^{A} is H, C₁₋₄alkyl, or haloalkyl;
R′ is H, C₁₋₄alkyl, or haloalkyl;
R″ is H, C₁₋₄alkyl, or haloalkyl; and
n is 0, 1, 2, or 3.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein L¹ is —O—.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein L¹ is —NR^{A}—. In one embodiment, R^{A} is H. In another embodiment, R^{A} is C₁₋₄alkyl. In another embodiment, R^{A} is methyl or ethyl. In one embodiment, R^{A} is C₁₋₄haloalkyl. In another embodiment, R^{A} is —CF₃ or —CH₂CF₃.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R¹ is H.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R¹ is C₁₋₄alkyl or C₁₋₄haloalkyl.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R¹ is halo.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R¹ is —CN.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R² is H.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R² is carbocycle or heterocycle. In one embodiment, R² is thiophenyl, thiazolyl, isthiazolyl, thiadiazolyl, furanyl, oxazolyl, isoxazolyl, oxadiazolyl, pyrrolyl, imidazolyl, pyrazolyl, or triazolyl. In another embodiment, R² is imidazolyl.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R³ is H.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R³ is —C₁₋₄alkyl-R^{3a}. In one embodiment, R^{3a} is H. In another embodiment, R^{3a} is —CN.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R³ is methyl or ethyl.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R³ is —CH₂CN or —CH₂CH₂CN.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R³ is —C₁₋₄haloalkyl-R^{3a}. In another embodiment, R^{3a} is H or halo. In one embodiment, R³ is —CH₂F, —CHF₂, —CF₃, —CF₂CF₃, —CH₂CH₂F, —CH₂CHF₂, or —CH₂CF₃.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R³ is C₂₋₄alkenyl. In one embodiment, R³ is:

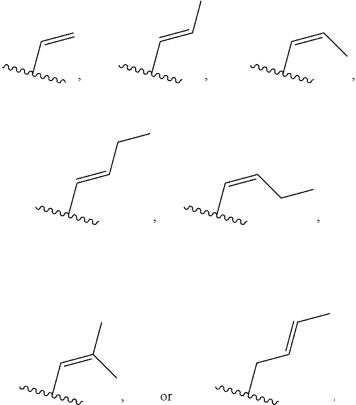

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R³ is C₂₋₄alkynyl. In one embodiment, R³ is:

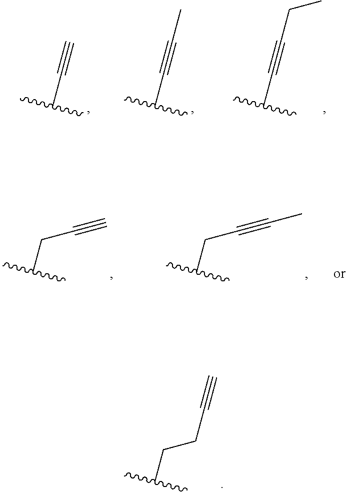

In still another embodiment, R³ is:

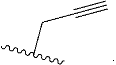 [SMILES: *CC#C]

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁵ is H.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁵ is halo. In one embodiment, R⁵ is Cl or Br.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁵ is C₁₋₄alkyl or C₁₋₄haloalkyl.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁵ is carbocycle or heterocycle. In one embodiment, R⁵ is phenyl.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁶ is H.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁶ is C₁₋₄alkyl. In one embodiment, R⁶ is methyl or ethyl.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁶ is C₃-6cycloalkyl or —C₁₋₄alkyl-C₃₋₆cycloalkyl. In one embodiment, R⁶ is cyclopropyl, cyclobutyl, —CH₂-cyclopropyl, or —CH₂-cyclobutyl.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁶ is —CN.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁷ is —NR^{7a}R^{7b}. In one embodiment, R^{7a} is H. In another embodiment, R^{7b} is H. In still another embodiment, R^{7a} is H and R^{7b} is H. In another embodiment, at least one of R^{7a} or R^{7b} is, independently, C₁₋₄alkyl. In still another embodiment, at least one of R⁷ or R^{7b} is, independently, —CN.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁷ is —C₁₋₄alkyl-R^{7c}. In one embodiment, R^{7c} is H. In another embodiment, R⁷ is methyl or ethyl.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁷ is —C₁₋₄alkyl-R^{7c} and R^{7c} is —CN. In another embodiment, R⁷ is —CH₂CN or —CH₂CH₂CN.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁷ is —C₁₋₄alkyl-R^{7c} and R^{7c} is:

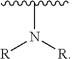

In one embodiment, R is, at each occurrence, independently H, C₁₋₄alkyl, C₁₋₄haloalkyl, carbocycle, heterocycle, —OR′, —C₁₋₄alkyl-OR′, —O—C₁₋₄alkyl, —C₁₋₄haloalkyl-OR′, —O—C₁₋₄haloalkyl, —O-carbocycle, —O-heterocycle, —C(O)R′, —C₁₋₄alkyl-C(O)R′, —C₁₋₄haloalkyl-C(O)R′, —C(O)-carbocycle, —C(O)-heterocycle, —C(O)OR′, —C₁₋₄alkyl-C(O)OR′, —C₁₋₄haloalkyl-C(O)OR′, —C(O)O-carbocycle, —C(O)O-heterocycle, —OC(O)R′, —C₁₋₄alkyl-OC(O)R′, —C₁₋₄haloalkyl-OC(O)R′, —OC(O)-carbocycle, or —OC(O)-heterocycle. In another embodiment, at least one R is H.

In one embodiment, a compound is provided having the structure of any one of Formulas (I), (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), or (XI-C), or a pharmaceutically acceptable salt, solvate, hydrate, isomer, tautomer, racemate, or isotope thereof, wherein R⁷ is —C₁₋₄alkyl-R^{7c} and R^{7c} is —C(O)OR′. In one embodiment, R^{7c} is —C(O)OR′ and R′ is H. In another embodiment, R^{7c} is —C(O)OR′ and R′ is C₁₋₄alkyl or C₁₋₄haloalkyl.

Representative compounds of Formula (I), and Formulas (II), (III), (III-A), (III-B), (III-C), (IV), (IV-A), (IV-B), (IV-C), (V), (V-A), (V-B), (V-C), (VI), (VI-A), (VI-B), (VI-C), (VII), (VIII), (VIII-A), (VIII-B), (VIII-C), (IX), (IX-A), (IX-B), (IX-C), (X), (X-A), (X-B), (X-C), (XI), (XI-A), (XI-B), and (XI-C) as applicable, include the compounds having the structure of those listed in Table 1, below, as well as pharmaceutically acceptable isomers, racemates, tautomers, hydrates, solvates, isotopes, or salts thereof. To this end, representative compounds are identified herein by their respective “Compound Number”, which is sometimes abbreviated as “Compound No.”, “Cmpd. No.” or “No.”

TABLE 1

| Cmpd. | | |
|---|---|---|
| No. | Structure | Name |
| 1A | 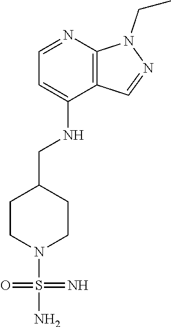 [SMILES: CCn1ncc2c(NCC3CCN(S(=N)(N)=O)CC3)ccnc21] | 4-(((1-ethyl-1H-pyrazolo[3,4-b]pyridin-4-yl) amino)methyl)piperidine-1-sulfonimidamide |
| 1B | 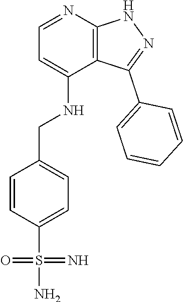 [SMILES: N=S(N)(=O)c1ccc(CNc2ccnc3[nH]nc(-c4ccccc4)c23)cc1] | 4-(((3-phenyl-1H-pyrazolo[3,4-b]pyridin-4-yl) amino)methyl)benzenesulfonimidamide |
| 1C | 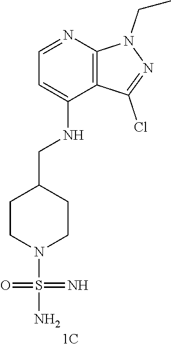 [SMILES: CCn1nc(Cl)c2c(NCC3CCN(S(=N)(N)=O)CC3)ccnc21] | 4-(((3-chloro-1-ethyl-1H-pyrazolo[3,4-b]pyridin- 4-yl)amino)methyl)piperidine-1-sulfonimidamide |
| 2A | 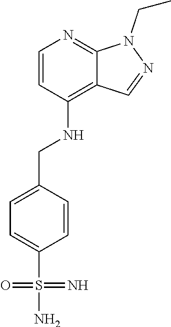 [SMILES: CCn1ncc2c(NCc3ccc(S(=N)(N)=O)cc3)ccnc21] | 4-(((1-ethyl-1H-pyrazolo[3,4-b]pyridin-4-yl) amino)methyl)benzene sulfonimidamide |
| 2B | 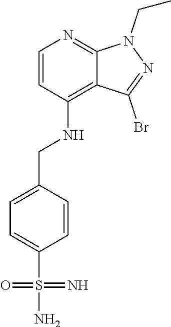 [SMILES: CCn1nc(Br)c2c(NCc3ccc(S(=N)(N)=O)cc3)ccnc21] | 4-(((3-bromo-1-ethyl-1H-pyrazolo[3,4-b]pyridin- 4-yl)amino)methyl)benzenesulfonimidamide |
| 2C | 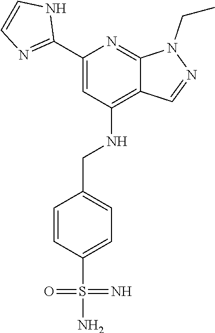 [SMILES: CCn1ncc2c(NCc3ccc(S(=N)(N)=O)cc3)cc(-c3ncc[nH]3)nc21] | 4-(((1-ethyl-6-(1H-imidazol-2-yl)-1H-pyrazolo [3,4-b]pyridin-4-yl)amino)methyl)benzene sulfonimidamide |
| 2D | 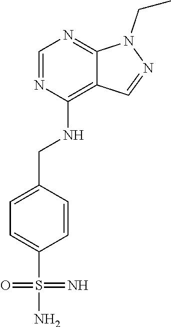 [SMILES: CCn1ncc2c(NCc3ccc(S(=N)(N)=O)cc3)ncnc21] | 4-(((1-ethyl-1H-pyrazolo[3,4-d]pyrimidin-4-yl) amino)methyl)benzenesulfonimidamide |
| 3A | 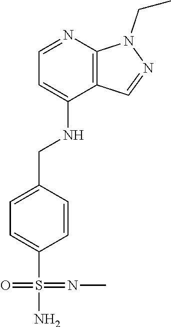 [SMILES: CCn1ncc2c(NCc3ccc(S(N)(=O)=NC)cc3)ccnc21] | 4-(((1-ethyl-1H-pyrazolo[3,4-b]pyridin-4-yl) amino)methyl)-N′- methylbenzenesulfonimidamide |
| 3B | 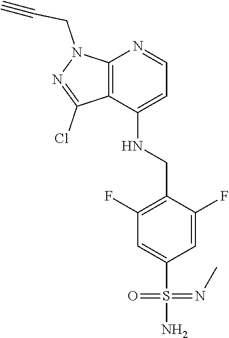 [SMILES: C#CCn1nc(Cl)c2c(NCc3c(F)cc(S(N)(=O)=NC)cc3F)ccnc21] | 4-(((3-chloro-1-(prop-2-yn-1-yl)-1H- pyrazolo[3,4-b]pyridin-4-yl)amino)methyl)-3,5- difluoro-N-methylbenzenesulfonimidamide |
| 3C | 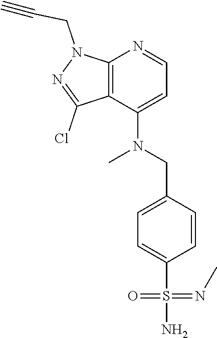 [SMILES: C#CCn1nc(Cl)c2c(N(C)Cc3ccc(S(N)(=O)=NC)cc3)ccnc21] | 4-(((3-chloro-1-(prop-2-yn-1-yl)-1H- pyrazolo[3,4-b]pyridin-4- yl)(methyl)amino)methyl)-N′- methylbenzenesulfonimidamide |
| 3D | 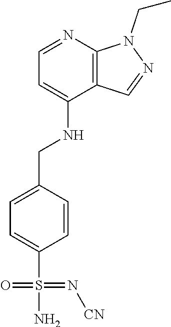 [SMILES: CCn1ncc2c(NCc3ccc(S(N)(=O)=NC#N)cc3)ccnc21] | N′-cyano-4-(((1-ethyl-1H-pyrazolo[3,4-b]pyridin- 4-yl)amino)methyl)benzenesulfonimidamide |
| 3E | 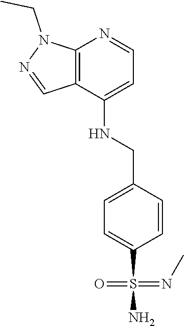 [SMILES: CCn1ncc2c(NCc3ccc([S@@](N)(=O)=NC)cc3)ccnc21] | (S)-4-(((1-ethyl-1H-pyrazolo[3,4-b]pyridin-4- yl)amino)methyl)-N′- methylbenzenesulfonimidamide |
| 3F | 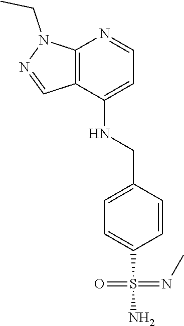 [SMILES: CCn1ncc2c(NCc3ccc([S@](N)(=O)=NC)cc3)ccnc21] | (R)-4-(((1-ethyl-1H-pyrazolo[3,4-b]pyridin-4- yl)amino)methyl)-N′- methylbenzenesulfonimidamide |
| 4A | 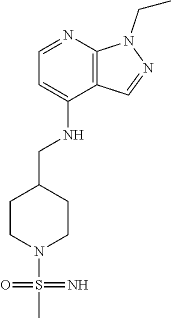 [SMILES: CCn1ncc2c(NCC3CCN(S(C)(=N)=O)CC3)ccnc21] | 1-ethyl-N-((1-(S-methylsulfonimidoyl)piperidin- 4-yl)methyl)-1H-pyrazolo[3,4-b]pyridin-4-amine |
| 5A | 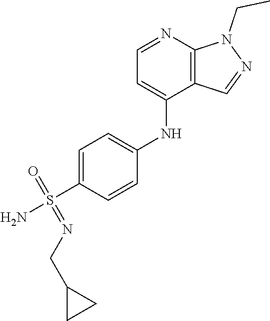 [SMILES: CCn1ncc2c(Nc3ccc(S(N)(=O)=NCC4CC4)cc3)ccnc21] | N′-(cyclopropylmethyl)-4-((1-ethyl-1H-pyrazolo [3,4-b]pyridin-4-yl)amino) benzenesulfonimidamide |
| 5B | 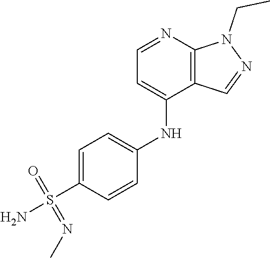 [SMILES: CCn1ncc2c(Nc3ccc(S(N)(=O)=NC)cc3)ccnc21] | 4-((1-ethyl-1H-pyrazolo[3,4-b]pyridin-4-yl) amino)-N′-methylbenzenesulfonimidamide |
| 5C | 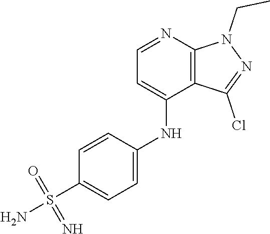 [SMILES: CCn1nc(Cl)c2c(Nc3ccc(S(=N)(N)=O)cc3)ccnc21] | 4-((3-chloro-1-ethyl-1H-pyrazolo[3,4-b]pyridin- 4-yl)amino)benzenesulfonimidamide |
| 5D | 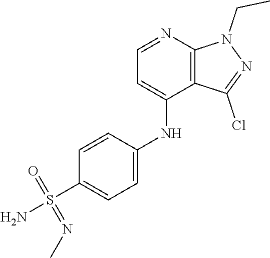 [SMILES: CCn1nc(Cl)c2c(Nc3ccc(S(N)(=O)=NC)cc3)ccnc21] | 4-((3-chloro-1-ethyl-1H-pyrazolo[3,4-b]pyridin- 4-yl)amino)-N′-methylbenzenesulfonimidamide |
| 5E | 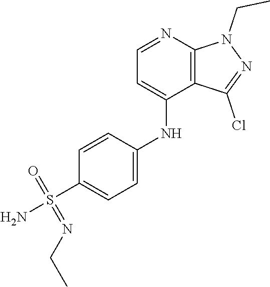 [SMILES: CCN=S(N)(=O)c1ccc(Nc2ccnc3c2c(Cl)nn3CC)cc1] | 4-((3-chloro-1-ethyl-1H-pyrazolo[3,4-b]pyridin- 4-yl)amino)-N′-ethylbenzenesulfonimidamide |
| 5F | 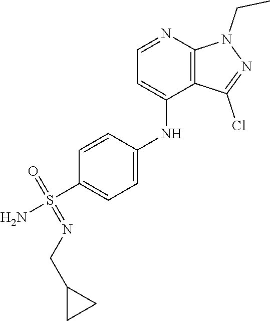 [SMILES: CCn1nc(Cl)c2c(Nc3ccc(S(N)(=O)=NCC4CC4)cc3)ccnc21] | 4-((3-chloro-1-ethyl-1H-pyrazolo[3,4-b]pyridin- 4-yl)amino)-N′-(cyclopropylmethyl)benzene sulfonimidamide |
| 5G | 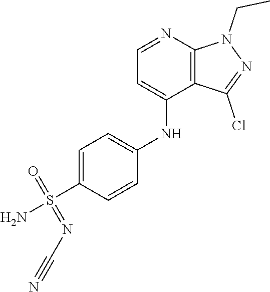 [SMILES: CCn1nc(Cl)c2c(Nc3ccc(S(N)(=O)=NC#N)cc3)ccnc21] | 4-((3-chloro-1-ethyl-1H-pyrazolo[3,4-b]pyridin- 4-yl)amino)-N′-cyanobenzenesulfonimidamide |
| 5H | 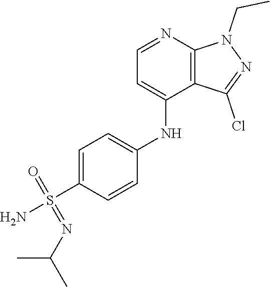 [SMILES: CCn1nc(Cl)c2c(Nc3ccc(S(N)(=O)=NC(C)C)cc3)ccnc21] | 4-((3-chloro-1-ethyl-1H-pyrazolo[3,4-b]pyridin- 4-yl)amino)-N′- isopropylbenzenesulfonimidamide |
| 5I | 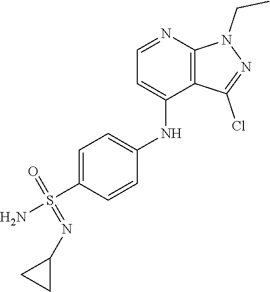 [SMILES: CCn1nc(Cl)c2c(Nc3ccc(S(N)(=O)=NC4CC4)cc3)ccnc21] | 4-((3-chloro-1-ethyl-1H-pyrazolo[3,4-b]pyridin- 4-yl)amino)-N′- cyclopropylbenzenesulfonimidamide |
| 6A | 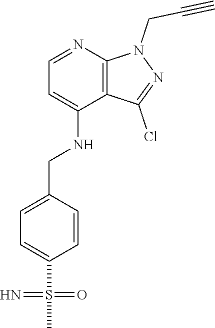 | (R)-(4-(((3-chloro-1-(prop-2-yn-1-yl)-1H- pyrazolo[3,4-b]pyridin-4-yl)amino)methyl) phenyl)(imino)(methyl)-λ⁶-sulfanone |
| 6B | 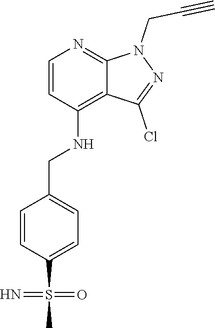 [SMILES: C#CCn1nc(Cl)c2c(NCc3ccc([S@@](C)(=N)=O)cc3)ccnc21] | (S)-(4-(((3-chloro-1-(prop-2-yn-1-yl)-1H- pyrazolo[3,4-b]pyridin-4-yl)amino)methyl) phenyl)(imino)(methyl)-λ⁶-sulfanone |
| 7A | 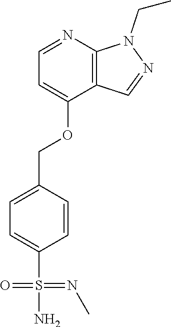 [SMILES: CCn1ncc2c(OCc3ccc(S(N)(=O)=NC)cc3)ccnc21] | 4-(((1-ethyl-1H-pyrazolo[3,4-b]pyridin-4-yl)oxy) methyl)-N′-methylbenzenesulfonimidamide |
| 8A | 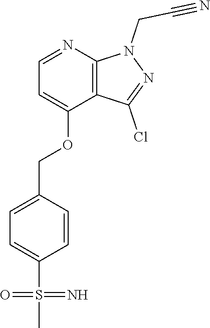 [SMILES: CS(=N)(=O)c1ccc(COc2ccnc3c2c(Cl)nn3CC#N)cc1] | 2-(3-chloro-4-((4-(S-methylsulfonimidoyl) benzyl)oxy)-1H-pyrazolo[3,4-b]pyridin-1-yl) acetonitrile |
| 9A | 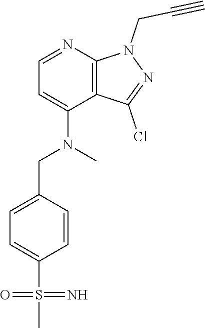 [SMILES: C#CCn1nc(Cl)c2c(N(C)Cc3ccc(S(C)(=N)=O)cc3)ccnc21] | (4-(((3-chloro-1-(prop-2-yn-1-yl)-1H-pyrazolo [3,4-b]pyridin-4-yl)(methyl)amino)methyl) phenyl)(imino)(methyl)-λ⁶-sulfanone |
| 9B | 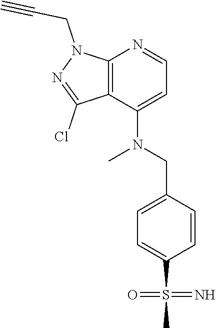 [SMILES: C#CCn1nc(Cl)c2c(N(C)Cc3ccc([S@](C)(=N)=O)cc3)ccnc21] | (R)-(4-(((3-chloro-1-(prop-2-yn-1-yl)-1H- pyrazolo[3,4-b]pyridin-4- yl)(methyl)amino)methyl)phenyl)(imino)(methyl)- λ⁶-sulfanone |
| 9C | 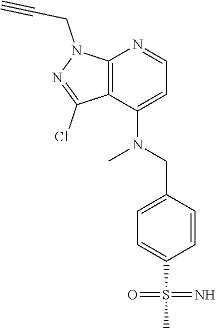 [SMILES: C#CCn1nc(Cl)c2c(N(C)Cc3ccc([S@@](C)(=N)=O)cc3)ccnc21] | (S)-(4-(((3-chloro-1-(prop-2-yn-1-yl)-1H- pyrazolo[3,4-b]pyridin-4- yl)(methyl)amino)methyl)phenyl)(imino)(methyl)- λ⁶-sulfanone |
| 9D | 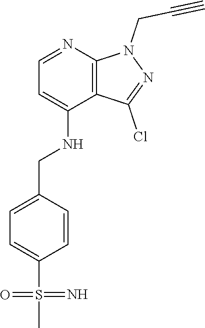 [SMILES: C#CCn1nc(Cl)c2c(NCc3ccc(S(C)(=N)=O)cc3)ccnc21] | (4-(((3-chloro-1-(prop-2-yn-1-yl)-1H- pyrazolo[3,4-b]pyridin-4- yl)amino)methyl)phenyl)(imino)(methyl)-λ6- sulfanone |
| 9E | 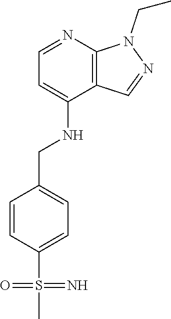 [SMILES: CCn1ncc2c(NCc3ccc(S(C)(=N)=O)cc3)ccnc21] | (4-(((1-ethyl-1H-pyrazolo[3,4-b]pyridin-4- yl)amino)methyl)phenyl)(imino)(methyl)-λ6- sulfanone |
| 9F | 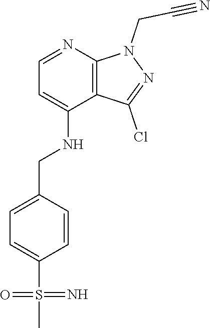 [SMILES: CS(=N)(=O)c1ccc(CNc2ccnc3c2c(Cl)nn3CC#N)cc1] | 2-(3-chloro-4-((4-(S- methylsulfonimidoyl)benzyl)amino)-1H- pyrazolo[3,4-b]pyridin-1-yl)acetonitrile |
| 9G | 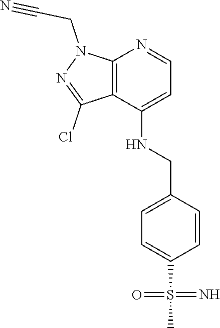 [SMILES: C[S@](=N)(=O)c1ccc(CNc2ccnc3c2c(Cl)nn3CC#N)cc1] | (S)-2-(3-chloro-4-((4-(S-methylsulfonimidoyl) benzyl)amino)-1H-pyrazolo[3,4-b]pyridin-1-yl) acetonitrile |
| 9H | 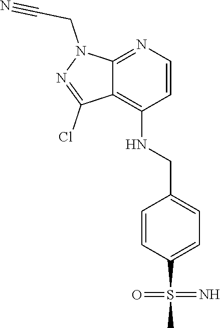 [SMILES: C[S@@](=N)(=O)c1ccc(CNc2ccnc3c2c(Cl)nn3CC#N)cc1] | (R)-2-(3-chloro-4-((4-(S- methylsulfonimidoyl)benzyl)amino)-1H- pyrazolo[3,4-b]pyridin-1-yl)acetonitrile |
| 9I | 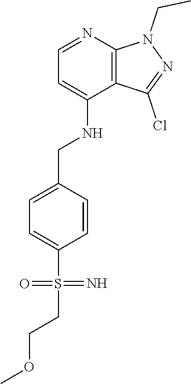 [SMILES: CCn1nc(Cl)c2c(NCc3ccc(S(=N)(=O)CCOC)cc3)ccnc21] | (4-(((3-chloro-1-ethyl-1H-pyrazolo[3,4- b]pyridin-4-yl)amino)methyl)phenyl)(imino)(2- methoxyethyl)-λ6-sulfanone |
| 9J | 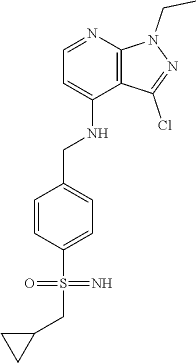 [SMILES: CCn1nc(Cl)c2c(NCc3ccc(S(=N)(=O)CC4CC4)cc3)ccnc21] | (4-(((3-chloro-1-ethyl-1H-pyrazolo[3,4-b] pyridin-4-yl)amino)methyl)phenyl) (cyclopropylmethyl)(imino)-λ⁶-sulfanone |
| 9K | 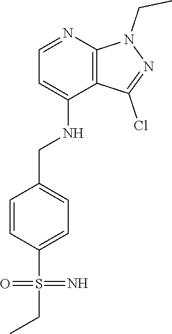 [SMILES: CCn1nc(Cl)c2c(NCc3ccc(S(=N)(=O)CC)cc3)ccnc21] | (4-(((3-chloro-1-ethyl-1H-pyrazolo[3,4-b] pyridine-4-yl)amino)methyl)phenyl)(ethyl) (imino)-λ⁶-sulfanone |
| 9L | 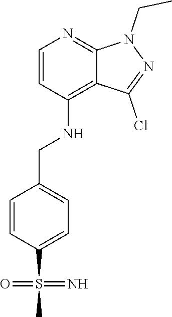 [SMILES: CCn1nc(Cl)c2c(NCc3ccc([S@](C)(=N)=O)cc3)ccnc21] | (R)-(4-(((3-chloro-1-ethyl-1H-pyrazolo[3,4-b] pyridin-4-yl)amino)methyl)phenyl)(imino) (methyl)-λ⁶-sulfanone |
| 9M | 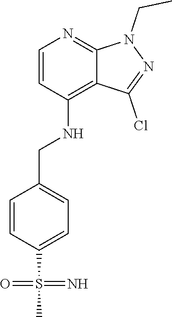 [SMILES: CCn1nc(Cl)c2c(NCc3ccc([S@@](C)(=N)=O)cc3)ccnc21] | (S)-(4-(((3-chloro-1-ethyl-1H-pyrazolo[3,4-b] pyridin-4-yl)amino)methyl)phenyl)(imino) (methyl)-λ⁶-sulfanone |
| 9N | 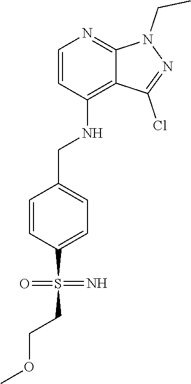 [SMILES: CCn1nc(Cl)c2c(NCc3ccc([S@](=N)(=O)CCOC)cc3)ccnc21] | R)-(4-(((3-chloro-1-ethyl-1H-pyrazolo[3,4-b] pyridin-4-yl)amino)methyl)phenyl)(imino)(2- methoxyethyl)-λ⁶-sulfanone |
| 9O | 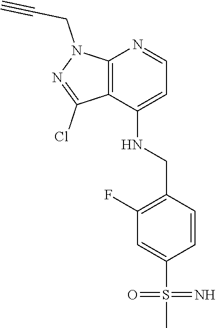 [SMILES: C#CCn1nc(Cl)c2c(NCc3ccc(S(C)(=N)=O)cc3F)ccnc21] | (S)-(4-(((3-chloro-1-ethyl-1H-pyrazolo[3,4-b] pyridin-4-yl)amino)methyl)phenyl)(imino)(2- methoxyethyl)-λ⁶-sulfanone |
| 9P | 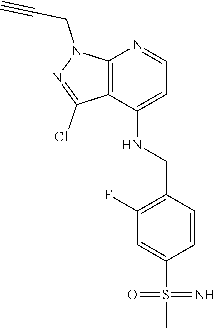 [SMILES: C#CCn1nc(Cl)c2c(NCc3ccc(S(C)(=N)=O)cc3F)ccnc21] | (4-(((3-chloro-1-(prop-2-yn-1-yl)-1H- pyrazolo[3,4-b]pyridine-4-yl)amino)methyl)-3- fluorophenyl)(imino)(methyl)-λ⁶-sulfanone |
| 9Q | 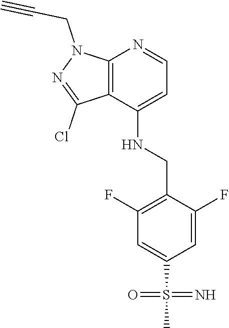 [SMILES: C#CCn1nc(Cl)c2c(NCc3c(F)cc([S@@](C)(=N)=O)cc3F)ccnc21] | (S)-(4-(((3-chloro-1-(prop-2-yn-1-yl)-1H- pyrazolo[3,4-b]pyridin-4-yl)amino)methyl)-3,5- difluorophenyl)(imino)(methyl)-λ⁶-sulfanone |
| 9R | 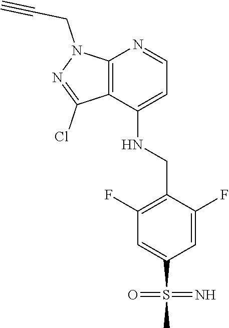 [SMILES: C#CCn1nc(Cl)c2c(NCc3c(F)cc([S@](C)(=N)=O)cc3F)ccnc21] | (R)-(4-(((3-chloro-1-(prop-2-yn-1-yl)-1H- pyrazolo[3,4-b]pyridin-4-yl)amino)methyl)-3,5- difluorophenyl)(imino)(methyl)-λ⁶-sulfanone |
| 9S | 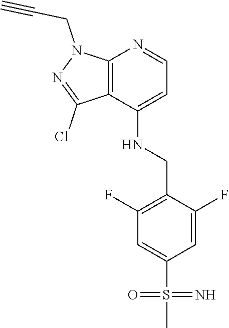 [SMILES: C#CCn1nc(Cl)c2c(NCc3c(F)cc(S(C)(=N)=O)cc3F)ccnc21] | (4-(((3-chloro-1-(prop-2-yn-1-yl)-1H- pyrazolo[3,4-b]pyridin-4-yl)amino)methyl)-3,5- difluorophenyl)(imino)(methyl)-λ⁶-sulfanone |
| 9T | 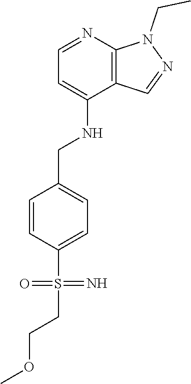 [SMILES: CCn1ncc2c(NCc3ccc(S(=N)(=O)CCOC)cc3)ccnc21] | (4-(((1-ethyl-1H-pyrazolo[3,4-b]pyridin-4- yl)amino)methyl)-phenyl)(imino)(2- methoxyethyl)-λ⁶-sulfanone |
| 10A | 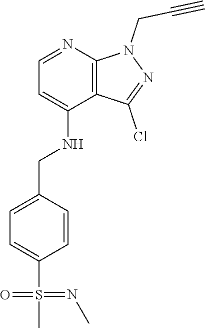 [SMILES: C#CCn1nc(Cl)c2c(NCc3ccc(S(C)(=O)=NC)cc3)ccnc21] | (4-(((3-chloro-1-(prop-2-yn-1-yl)-1H- pyrazolo[3,4-b]pyridin-4- yl)amino)methyl)phenyl)(methyl)(methylimino)- λ6-sulfanone |
| 10B | 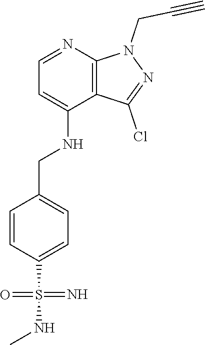 [SMILES: C#CCn1nc(Cl)c2c(NCc3ccc([S@@](=N)(=O)NC)cc3)ccnc21] | (R)-4-(((3-chloro-1-(prop-2-yn-1-yl)-1H- pyrazolo[3,4-b]pyridin-4-yl)amino)methyl)-N- methylbenzenesulfonimidamide |
| 10C | 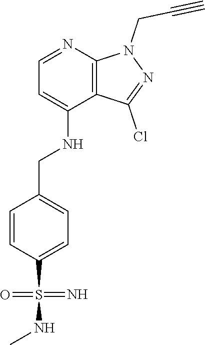 [SMILES: C#CCn1nc(Cl)c2c(NCc3ccc([S@](=N)(=O)NC)cc3)ccnc21] | (S)-4-(((3-chloro-1-(prop-2-yn-1-yl)-1H- pyrazolo[3,4-b]pyridin-4-yl)amino)methyl)-N- methylbenzenesulfonimidamide |
| 10D | 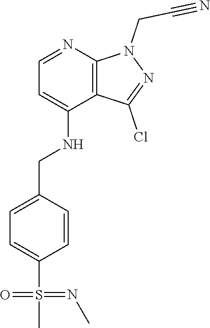 [SMILES: CN=S(C)(=O)c1ccc(CNc2ccnc3c2c(Cl)nn3CC#N)cc1] | 2-(3-chloro-4-((4-(N,S- dimethylsulfonimidoyl)benzyl)amino)-1H- pyrazolo[3,4-b]pyridin-1-yl)acetonitrile |
| 10E | 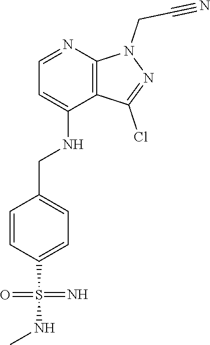 [SMILES: CN[S@](=N)(=O)c1ccc(CNc2ccnc3c2c(Cl)nn3CC#N)cc1] | (S)-4-(((3-chloro-1-(cyanomethyl)-1H- pyrazolo[3,4-b]pyridin-4-yl)amino)methyl)-N- methylbenzenesulfonimidamide |
| 10F | 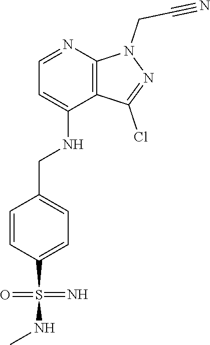 [SMILES: CN[S@@](=N)(=O)c1ccc(CNc2ccnc3c2c(Cl)nn3CC#N)cc1] | (R)-4-(((3-chloro-1-(cyanomethyl)-1H- pyrazolo[3,4-b]pyridin-4-yl)amino)methyl)-N- methylbenzenesulfonimidamide |
| 11A | 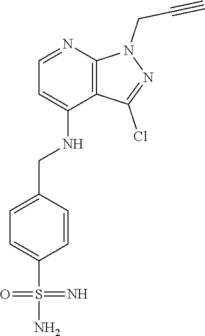 [SMILES: C#CCn1nc(Cl)c2c(NCc3ccc(S(=N)(N)=O)cc3)ccnc21] | 4-(((3-chloro-1-(prop-2-yn-1-yl)-1H- pyrazolo[3,4-b]pyridin-4- yl)amino)methyl)benzenesulfonimidamide |
| 11B | 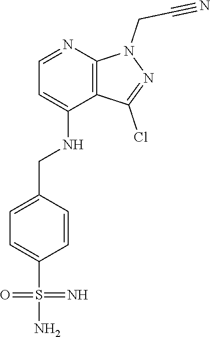 [SMILES: N#CCn1nc(Cl)c2c(NCc3ccc(S(=N)(N)=O)cc3)ccnc21] | 4-(((3-chloro-1-(cyanomethyl)-1H-pyrazolo[3,4- b]pyridin-4- yl)amino)methyl)benzenesulfonimidamide |
| 11C | 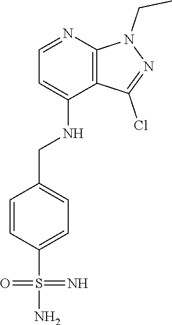 [SMILES: CCn1nc(Cl)c2c(NCc3ccc(S(=N)(N)=O)cc3)ccnc21] | 4-(((3-chloro-1-ethyl-1H-pyrazolo[3,4-b]pyridin- 4-yl)amino)methyl)benzenesulfonimidamide |
| 12A | 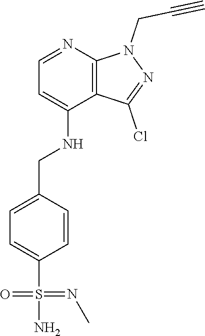 [SMILES: C#CCn1nc(Cl)c2c(NCc3ccc(S(N)(=O)=NC)cc3)ccnc21] | 4-(((3-chloro-1-(prop-2-yn-1-yl)-1H- pyrazolo[3,4-b]pyridin-4-yl)amino)methyl)-N′- methylbenzenesulfonimidamide |
| 12B | 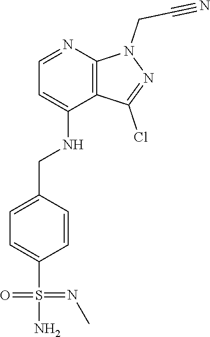 [SMILES: CN=S(N)(=O)c1ccc(CNc2ccnc3c2c(Cl)nn3CC#N)cc1] | 4-(((3-chloro-1-(cyanomethyl)-1H-pyrazolo[3,4- b]pyridin-4-yl)amino)methyl)-N′- methylbenzenesulfonimidamide |
| 12C | 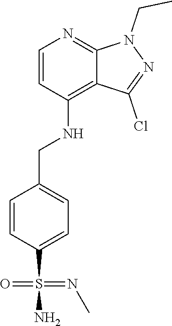 [SMILES: CCn1nc(Cl)c2c(NCc3ccc([S@@](N)(=O)=NC)cc3)ccnc21] | (S)-4-(((3-chloro-1-ethyl-1H-pyrazolo[3,4-b] pyridin-4-yl)amino)methyl)-N′-methyl benzenesulfonimidamide |
| 12D | 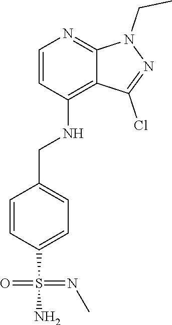 [SMILES: CCn1nc(Cl)c2c(NCc3ccc([S@](N)(=O)=NC)cc3)ccnc21] | (R)-4-(((3-chloro-1-ethyl-1H-pyrazolo[3,4-b] pyridin-4-yl)amino)methyl)-N′-methyl benzenesulfonimidamide |
| 12E | 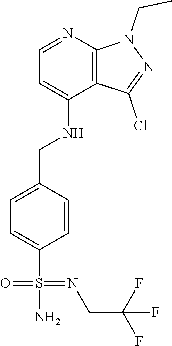 [SMILES: CCn1nc(Cl)c2c(NCc3ccc(S(N)(=O)=NCC(F)(F)F)cc3)ccnc21] | 4-(((3-chloro-1-ethyl-1H-pyrazolo[3,4-b]pyridin- 4-yl)amino)methyl)-N′-(2,2,2-trifluoroethyl) benzenesulfonimidamide |
| 12F | 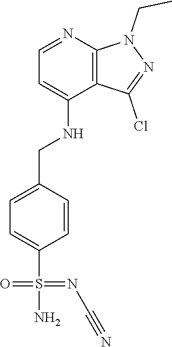 [SMILES: CCn1nc(Cl)c2c(NCc3ccc(S(N)(=O)=NC#N)cc3)ccnc21] | 4-(((3-chloro-1-ethyl-1H-pyrazolo[3,4-b]pyridin- 4-yl)amino)methyl)-N′-(2,2,2-trifluoroethyl) benzenesulfonimidamide |
| 12G | 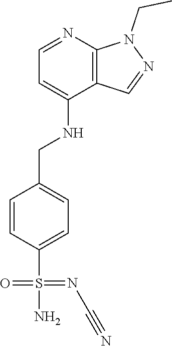 [SMILES: CCn1ncc2c(NCc3ccc(S(N)(=O)=NC#N)cc3)ccnc21] | N′-cyano-4-(((1-ethyl-1H-pyrazolo[3,4- b]pyridin-4- yl)amino)methyl)benzenesulfonimidamide |
| 12H | 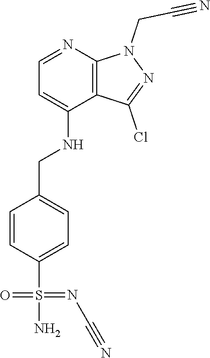 [SMILES: N#CCn1nc(Cl)c2c(NCc3ccc(S(N)(=O)=NC#N)cc3)ccnc21] | 4-(((3-chloro-1-(cyanomethyl)-1H-pyrazolo[3,4- b]pyridin-4-yl)amino)methyl)-N′- cyanobenzenesulfonimidamide |
| 12I | 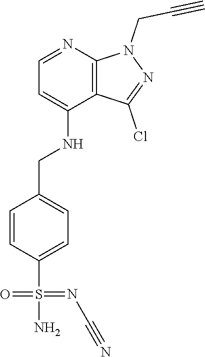 [SMILES: C#CCn1nc(Cl)c2c(NCc3ccc(S(N)(=O)=NC#N)cc3)ccnc21] | 4-(((3-chloro-1-(prop-2-yn-1-yl)-1H- pyrazolo[3,4-b]pyridin-4-yl)amino)methyl)-N′- cyanobenzenesulfonimidamide |
| 12J | 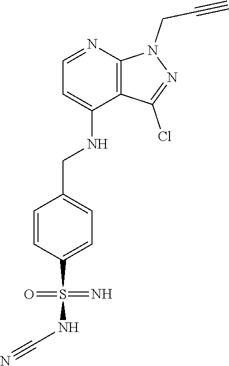 [SMILES: C#CCn1nc(Cl)c2c(NCc3ccc([S@](=N)(=O)NC#N)cc3)ccnc21] | (R)-4-(((3-chloro-1-(prop-2-yn-1-yl)-1H- pyrazolo[3,4-b]pyridin-4-yl)amino)methyl)-N- cyanobenzenesulfonimidamide |
| 12K | 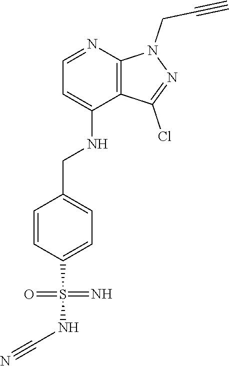 [SMILES: C#CCn1nc(Cl)c2c(NCc3ccc([S@@](=N)(=O)NC#N)cc3)ccnc21] | (S)-4-(((3-chloro-1-(prop-2-yn-1-yl)-1H- pyrazolo[3,4-b]pyridin-4-yl)amino)methyl)-N- cyanobenzenesulfonimidamide |
| 13A | 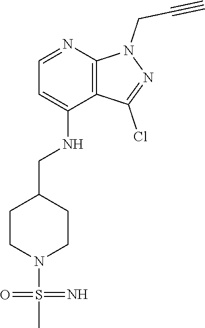 [SMILES: C#CCn1nc(Cl)c2c(NCC3CCN(S(C)(=N)=O)CC3)ccnc21] | 3-chloro-N-((1-(S- methylsulfonimidoyl)piperidin-4-yl)methyl)-1- (prop-2-yn-1-yl)-1H-pyrazolo[3,4-b]pyridin-4- amine |
| 13B | 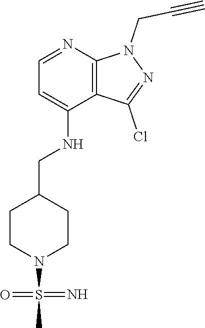 [SMILES: C#CCn1nc(Cl)c2c(NCC3CCN([S@](C)(=N)=O)CC3)ccnc21] | (S)-3-chloro-N-((1-(S- methylsulfonimidoyl)piperidin-4-yl)methyl)-1- (prop-2-yn-1-yl)-1H-pyrazolo[3,4-b]pyridin-4- amine |
| 13C | 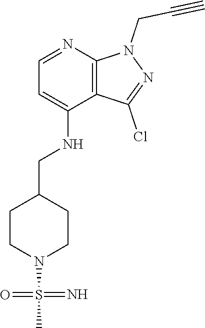 [SMILES: C#CCn1nc(Cl)c2c(NCC3CCN([S@@](C)(=N)=O)CC3)ccnc21] | (R)-3-chloro-N-((1-(S- methylsulfonimidoyl)piperidin-4-yl)methyl)-1- (prop-2-yn-1-yl)-1H-pyrazolo[3,4-b]pyridin-4- amine |
| 13D | 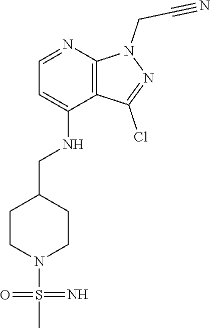 [SMILES: CS(=N)(=O)N1CCC(CNc2ccnc3c2c(Cl)nn3CC#N)CC1] | 2-(3-chloro-4-(((1-(S- methylsulfonimidoyl)piperidin-4- yl)methyl)amino)-1H-pyrazolo[3,4-b] pyridin-1- yl)acetonitrile |
| 13E | 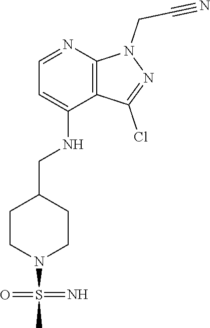 [SMILES: C[S@@](=N)(=O)N1CCC(CNc2ccnc3c2c(Cl)nn3CC#N)CC1] | (S)-2-(3-chloro-4-(((1-(S- methylsulfonimidoyl)piperidin-4- yl)methyl)amino)-1H-pyrazolo[3,4-b] pyridin-1- yl)acetonitrile |
| 13F | 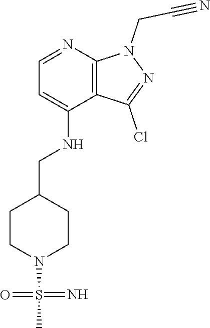 [SMILES: C[S@](=N)(=O)N1CCC(CNc2ccnc3c2c(Cl)nn3CC#N)CC1] | (R)-2-(3-chloro-4-(((1-(S- methylsulfonimidoyl)piperidin-4- yl)methyl)amino)-1H-pyrazolo[3,4-b]pyridin-1- yl)acetonitrile |
| 13G | 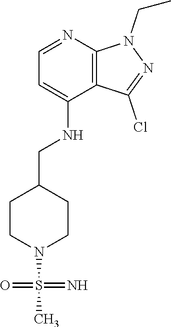 [SMILES: CCn1nc(Cl)c2c(NCC3CCN([S@@](C)(=N)=O)CC3)ccnc21] | (R)-3-chloro-1-ethyl-N-((1-(S- methylsulfonimidoyl)piperidin-4-yl)methyl)-1H- pyrazolo[3,4-b]pyridin-4-amine |
| 13H | 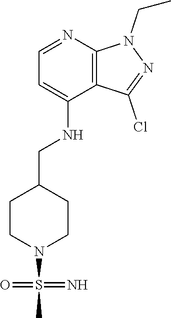 [SMILES: CCn1nc(Cl)c2c(NCC3CCN([S@](C)(=N)=O)CC3)ccnc21] | (S)-3-chloro-1-ethyl-N-((1-(S- methylsulfonimidoyl)piperidin-4-yl)methyl)-1H- pyrazolo[3,4-b]pyridin-4-amine |
| 14A | 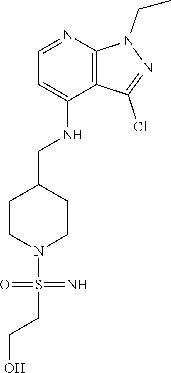 [SMILES: CCn1nc(Cl)c2c(NCC3CCN(S(=N)(=O)CCO)CC3)ccnc21] | 2-(4-(((3-chloro-1-ethyl-1H-pyrazolo[3,4- b]pyridin-4-yl)amino)methyl)piperidine-1- sulfonimidoyl)ethan-1-ol |
| 14B | 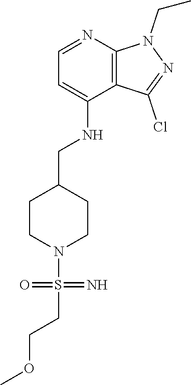 [SMILES: CCn1nc(Cl)c2c(NCC3CCN(S(=N)(=O)CCOC)CC3)ccnc21] | 3-chloro-1-ethyl-N-((1-(2- methoxyethylsulfonimidoyl)piperidin-4- yl)methyl)-1H-pyrazolo[3,4-b]pyridin-4-amine |
| 14C | 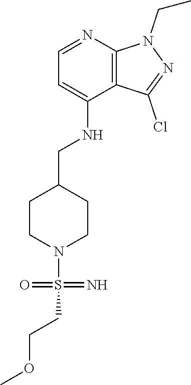 [SMILES: CCn1nc(Cl)c2c(NCC3CCN([S@@](=N)(=O)CCOC)CC3)ccnc21] | (R)-3-chloro-1-ethyl-N-((1-(2- methoxyethylsulfonimidoyl)piperidin-4-yl) methyl)-1H-pyrazolo[3,4-b]pyridin-4-amine |
| 14D | 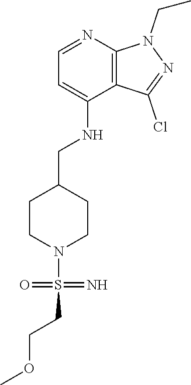 [SMILES: CCn1nc(Cl)c2c(NCC3CCN([S@](=N)(=O)CCOC)CC3)ccnc21] | (S)-3-chloro-1-ethyl-N-((1-(2- methoxyethylsulfonimidoyl)piperidin-4-yl) methyl)-1H-pyrazolo[3,4-b]pyridin-4-amine |
| 14E | 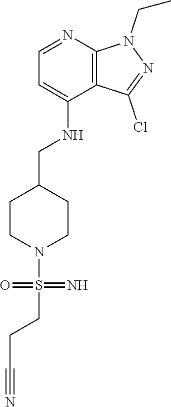 [SMILES: CCn1nc(Cl)c2c(NCC3CCN(S(=N)(=O)CCC#N)CC3)ccnc21] | 3-(4-(((3-chloro-1-ethyl-1H-pyrazolo[3,4-b] pyridin-4-yl)amino)methyl)piperidine-1- sulfonimidoyl)propanenitrile |
| 14F | 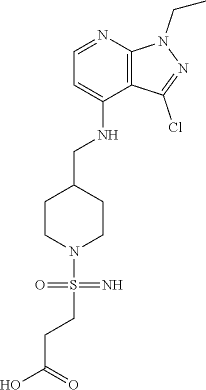 [SMILES: CCn1nc(Cl)c2c(NCC3CCN(S(=N)(=O)CCC(=O)O)CC3)ccnc21] | 3-(4-(((3-chloro-1-ethyl-1H-pyrazolo[3,4-b] pyridin-4-yl)amino)methyl)piperidine-1- sulfonimidoyl)propanoic acid |
| 14G | 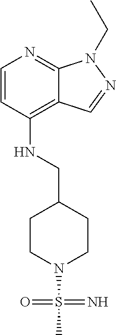 [SMILES: CCn1ncc2c(NCC3CCN([S@@](C)(=N)=O)CC3)ccnc21] | I-1-ethyl-N-((1-(S- methylsulfonimidoyl)piperidin-4-yl)methyl)-1H- pyrazolo[3,4-b]pyridin-4-amine |
| 14H | 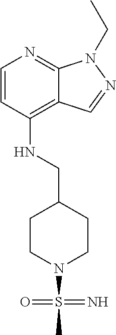 [SMILES: CCn1ncc2c(NCC3CCN([S@](C)(=N)=O)CC3)ccnc21] | (S)-1-ethyl-N-((1-(S- methylsulfonimidoyl)piperidin-4-yl)methyl)-1H- pyrazolo[3,4-b]pyridin-4-amine |
| 14I | 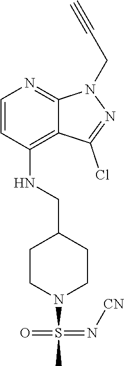 [SMILES: C#CCn1nc(Cl)c2c(NCC3CCN([S@@](C)(=O)=NC#N)CC3)ccnc21] | (S)-N-((4-(((3-chloro-1-(prop-2-yn-1-yl)-1H- pyrazolo[3,4-b]pyridin-4- yl)amino)methyl)piperidin-1-yl)(methyl)(oxo)- λ6-sulfaneylidene)cyanamide |
| 14J | 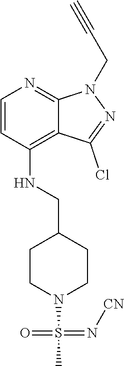 [SMILES: C#CCn1nc(Cl)c2c(NCC3CCN([S@](C)(=O)=NC#N)CC3)ccnc21] | (R)-N-((4-(((3-chloro-1-(prop-2-yn-1-yl)-1H- pyrazolo[3,4-b]pyridin-4- yl)amino)methyl)piperidin-1-yl)(methyl)(oxo)- λ6-sulfaneylidene)cyanamide |
| 14K | 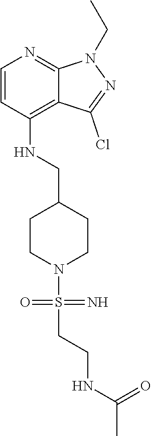 [SMILES: CCn1nc(Cl)c2c(NCC3CCN(S(=N)(=O)CCNC(C)=O)CC3)ccnc21] | N-(2-(4-(((3-chloro-1-ethyl-1H-pyrazolo[3,4-b] pyridin-4-yl)amino)methyl)piperidine-1- sulfonimidoyl)ethyl)acetamide |
| 15A | 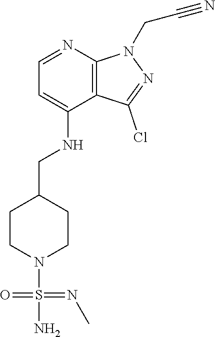 [SMILES: CN=S(N)(=O)N1CCC(CNc2ccnc3c2c(Cl)nn3CC#N)CC1] | 4-(((3-chloro-1-(cyanomethyl)-1H-pyrazolo[3,4- b]pyridin-4-yl)amino)methyl)-N′- methylpiperidine-1-sulfonimidamide |
| 15B | 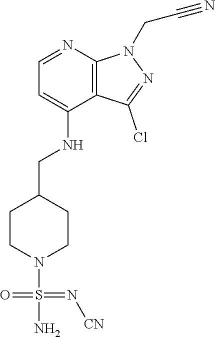 [SMILES: N#CCn1nc(Cl)c2c(NCC3CCN(S(N)(=O)=NC#N)CC3)ccnc21] | 4-(((3-chloro-1-(cyanomethyl)-1H-pyrazolo[3,4- b]pyridin-4-yl)amino)methyl)-N′- cyanopiperidine-1-sulfonimidamide |
| 15C | 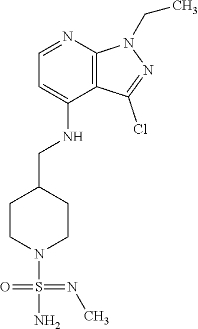 [SMILES: CCn1nc(Cl)c2c(NCC3CCN(S(N)(=O)=NC)CC3)ccnc21] | 4-(((3-chloro-1-ethyl-1H-pyrazolo[3,4-b]pyridin- 4-yl)amino)methyl)-N′-methylpiperidine-1- sulfonimidamide |
| 15D | 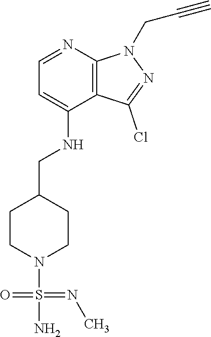 [SMILES: C#CCn1nc(Cl)c2c(NCC3CCN(S(N)(=O)=NC)CC3)ccnc21] | 4-(((3-chloro-1-(prop-2-yn-1-yl)-1H- pyrazolo[3,4-b]pyridine-4-yl)amino)methyl)-N′- methylpiperidine-1-sulfonimidamide |
| 15E | 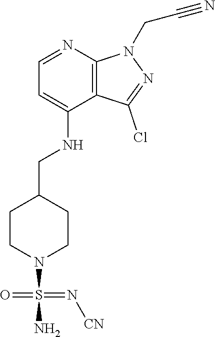 [SMILES: N#CCn1nc(Cl)c2c(NCC3CCN([S@@](N)(=O)=NC#N)CC3)ccnc21] | (S)-4-(((3-chloro-1-(cyanomethyl)-1H- pyrazolo[3,4-b]pyridine-4-yl)amino)methyl)-N′- cyanopiperidine-1-sulfonimidamide |
| 15F | 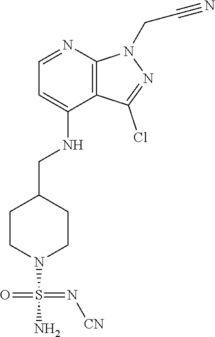 [SMILES: N#CCn1nc(Cl)c2c(NCC3CCN([S@](N)(=O)=NC#N)CC3)ccnc21] | (R)-4-(((3-chloro-1-(cyanomethyl)-1H- pyrazolo[3,4-b]pyridin-4-yl)amino)methyl)-N′- cyanopiperidine-1-sulfonimidamide |
| 15G | 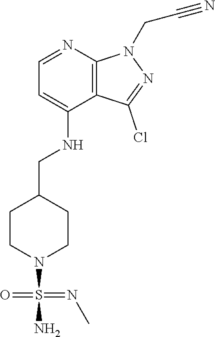 [SMILES: CN=[S@](N)(=O)N1CCC(CNc2ccnc3c2c(Cl)nn3CC#N)CC1] | (S)-4-(((3-chloro-1-(cyanomethyl)-1H- pyrazolo[3,4-b]pyridin-4-yl)amino)methyl)-N′- methylpiperidine-1-sulfonimidamide |
| 15H | 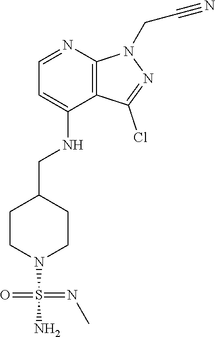 [SMILES: CN=[S@@](N)(=O)N1CCC(CNc2ccnc3c2c(Cl)nn3CC#N)CC1] | (R)-4-(((3-chloro-1-(cyanomethyl)-1H- pyrazolo[3,4-b]pyridin-4-yl)amino)methyl)-N′- methylpiperidine-1-sulfonimidamide |
| 15I | 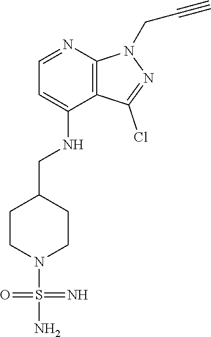 [SMILES: C#CCn1nc(Cl)c2c(NCC3CCN(S(=N)(N)=O)CC3)ccnc21] | 4-(((3-chloro-1-(prop-2-yn-1-yl)-1H- pyrazolo[3,4-b]pyridin-4- yl)amino)methyl)piperidine-1-sulfonimidamide |
| 16A | 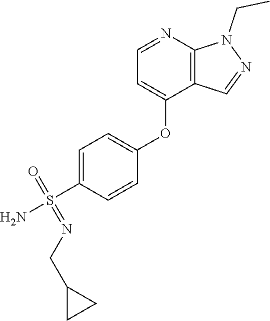 [SMILES: CCn1ncc2c(Oc3ccc(S(N)(=O)=NCC4CC4)cc3)ccnc21] | N′-(cyclopropylmethyl)-4-((1-ethyl-1H-pyrazolo [3,4-b]pyridin-4-yl)oxy)benzenesulfonimidamide |

“Isomer” is used herein to encompass all chiral, diastereomeric or racemic forms of a structure, unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized to be substantially free of their enantiomeric or diastereomeric partners, and these are all within the scope of certain embodiments of the disclosure. The isomers resulting from the presence of a chiral center comprise a pair of non-superimposable isomers that are called “enantiomers.” Single enantiomers of a pure compound are optically active (i.e., they can rotate the plane of plane polarized light and designated R or S). The term also encompasses isomers arising from substitution patterns across double bonds, in particular (R)- and (Z)-isomers, or cis- and trans-isomers. E-Z configuration describes the absolute stereochemistry across double bonds having two, three or four substituents. Following the Cahn-Ingold-Prelog priority rules (CIP rules), each substituent on a double bond is assigned a priority, and the positions of the higher of the two substituents on each carbon determined. If the two groups of higher priority are on the same side of the double bond (cis to each other), the bond is assigned Z (“zusammen”, German for “together”). If the two groups of higher priority are on opposite sides of the double bond (trans to each other), the bond is assigned E (“entgegen”, German for “opposite”). Each isomer may be isolated separately or exist as mixtures. The mixtures may be predominantly one isomer, e.g., 99.9%, or 99% or 90%, predominantly the other isomer, enriched in one or the other of the isomer (e.g., an 80/20 mixture, or a 40/60 mixture), or be approximately equal mixtures.

“Isolated optical isomer” means a compound which has been substantially purified from the corresponding optical isomer(s) of the same formula. For example, the isolated isomer may be at least about 80%, at least 80% or at least 85% pure. In other embodiments, the isolated isomer is at least 90% pure or at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5% or 99.9%.

The terms “racemate” and “racemic mixture” refer to an equal mixture of two enantiomers. A racemate is labeled “(±)” because it is not optically active (i.e., will not rotate plane-polarized light in either direction since its constituent enantiomers cancel each other out).

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other that water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound of Formulas (I) includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has several isotopes, fluorine 19 is longest-lived. Thus, an isotope of a compound having the structure of Formulas (I) includes, but not limited to, compounds of Formulas (I) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

The term “pharmaceutically acceptable” refers an agent that has been approved for human consumption and is generally non-toxic. For example, the term “pharmaceutically acceptable salt” refers to nontoxic inorganic or organic acid and/or base addition salts (see, e.g., Lit et al., Salt Selection for Basic Drugs, Int. J. Pharm., 33, 201-217, 1986) (incorporated by reference herein).

Pharmaceutically acceptable base addition salts of compounds of the disclosure include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′ dibenzylethylenediamine, chloroprocaine, choline, diethanolamine, ethylenediamine, meglumine (N-methylglucamine), and procaine.

Pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, βhydroxybutyric, salicylic, -galactaric, and galacturonic acid.

Although pharmaceutically unacceptable salts are not generally useful as medicaments, such salts may be useful, for example as intermediates in the synthesis of the compounds described herein, for example in their purification by recrystallization.

In some embodiments, the compounds are pharmaceutically acceptable salts. In some embodiments, the compounds are isomers. In some embodiments, the compounds are racemates. In some embodiments, the compounds are solvating. In some embodiments, the compounds are hydrates. In some embodiments, the compounds are isotopes.

### Pharmaceutical Compositions

Also disclosed herein are pharmaceutical compositions comprising a compound as described herein, or a pharmaceutically acceptable salt, solvate, hydrate, isomer, tautomer, racemate, or isotope thereof. In some embodiments, the pharmaceutical compositions further comprise a pharmaceutically acceptable carrier, diluent, or excipient. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid, or lower alkyl ethers of cellulose, silicic acid, fatty acids, fatty acid amines, fatty acid monoglycerides and diglycerides, pentaerythritol fatty acid esters, polyoxyethylene, hydroxymethylcellulose, and polyvinylpyrrolidone. Similarly, the carrier or diluent can include any sustained release material known in the art, such as glyceryl monostearate or glyceryl distearate, alone or mixed with a wax.

As used herein, the term “pharmaceutical composition” refers to a composition containing one or more of the compounds described herein, or a pharmaceutically acceptable isomer, racemate, hydrate, solvate, homolog or salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein. Conventional procedures and ingredients for the selection and preparation of suitable formulations are described, for example, in Remington: The Science and Practice of Pharmacy, 21^{st} Ed., Gennaro, Ed., Lippencott Williams & Wilkins (2005) and in The United States Pharmacopeia: The National Formulary (USP 36 NF31), published in 2013.

In other embodiments, there are provided methods of making a composition of a compound described herein including formulating a compound of the disclosure with a pharmaceutically acceptable carrier or diluent. In some embodiments, the pharmaceutically acceptable carrier or diluent is suitable for oral administration. In some such embodiments, the methods can further include the step of formulating the composition into a tablet or capsule. In other embodiments, the pharmaceutically acceptable carrier or diluent is suitable for parenteral administration. In some such embodiments, the methods further include the step of lyophilizing the composition to form a lyophilized preparation.

As used herein, the term “pharmaceutically acceptable carrier” refers to any ingredient other than the disclosed compounds, or a pharmaceutically acceptable isomer, racemate, hydrate, solvate, homolog or salt thereof (e.g., a carrier capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The formulations can be mixed with auxiliary agents which do not deleteriously react with the active compounds. Such additives can include wetting agents, emulsifying, and suspending agents, salt for influencing osmotic pressure, buffers and/or coloring substances, preserving agents, sweetening agents, or flavoring agents. The compositions can also be sterilized if desired.

The route of administration can be any route which effectively transports the active compound of the disclosure to the appropriate or desired site of action, such as oral, nasal, pulmonary, buccal, subdermal, intradermal, transdermal, or parenteral, e.g., rectal, depot, subcutaneous, intravenous, intraurethral, intramuscular, intranasal, ophthalmic solution, or an ointment, the oral route being preferred.

Dosage forms can be administered once a day, or more than once a day, such as twice or thrice daily. Alternatively, dosage forms can be administered less frequently than daily, such as every other day, or weekly, if found to be advisable by a prescribing physician. Dosing regimens include, for example, dose titration to the extent necessary or useful for the indication to be treated, thus allowing the patient's body to adapt to the treatment and/or to minimize or avoid unwanted side effects associated with the treatment. Other dosage forms include delayed or controlled-release forms. Suitable dosage regimens and/or forms include those set out, for example, in the latest edition of the Physicians' Desk Reference, incorporated herein by reference.

Inhibiting ENPP1 Activity and Treating Diseases Associated with ENPP1

In certain embodiments, described herein, are methods for inhibiting the activity of at least one ectonucleotide pyrophosphatase/phosphodiesterase 1 comprising contacting ENPP1 with a compound as described herein, or a pharmaceutically acceptable salt, solvate, hydrate, isomer, tautomer, racemate, isotope, or composition thereof. In certain embodiments, methods of treating a subject having a disease or disorder associated with the activity of ENPP1 are disclosed, the method comprising administering to a subject in need thereof a pharmaceutically effective amount of a compound as described herein, or a pharmaceutically acceptable salt, solvate, hydrate, isomer, tautomer, racemate, isotope, or composition thereof.

As used herein, the term “administering” or “administration” refers to providing a compound, a pharmaceutical composition comprising the same, to a subject by any acceptable means or route, including (for example) by oral, parenteral (e.g., intravenous), or topical administration.

As used herein, the term “treatment” refers to an intervention that ameliorates a sign or symptom of a disease or pathological condition. As used herein, the terms “treatment”, “treat” and “treating,” with reference to a disease, pathological condition, or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed. The terms cover the treatment of a disease-state in a mammal, particularly in a human, and include:
(a) preventing the disease-state from occurring in a mammal, in particular, when such mammal is predisposed to the disease state but has not yet been diagnosed as having it;
(b) inhibiting the disease-state, i.e., arresting its development; and/or
(c) relieving the disease-state, i.e., causing regression of the disease state.

As used herein, the term “ENPP1-mediated” or “ENPP1-modulated” or “ENPP1-dependent” diseases or disorders means any disease or other deleterious condition in which ENPP1, or a mutant thereof, is known to play a role. Accordingly, another embodiment of the present application relates to treating or lessening the severity of one or more diseases in which ENPP1, or a mutant thereof, are known to play a role. Specifically, the present application relates to a method of treating or lessening the severity of a disease or condition selected from an infection (including but not limited to bacterial infections and viral infections), proliferative disorders (such as cancer or a tumor), cardiovascular disease (including cardiac injury), diabetes or insulin resistance, chondrocalcinosis, calcium pyrophosphate deposition disorder (pseudogout), or hypophosphatasia, wherein said method comprises administering to a patient in need thereof a compound of Formula (I), or a pharmaceutically acceptable salt, solvate, hydrate, isomer, tautomer, racemate, isotope, or composition thereof, according to the present application.

As used herein, the term “subject” refers to an animal (e.g., a mammal, such as a human). A subject to be treated according to the methods described herein may be one who has been diagnosed with a viral infection or proliferative disorder, such as cancer or a tumor. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the term “effective amount” refers to a quantity of a specified agent sufficient to achieve a desired effect in a subject being treated with that agent. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure.

As used herein, the term “therapeutically effective amount” or “pharmaceutically effective amount” is intended to include an amount of a compound of the present invention alone or an amount of a compound of the present invention in combination with other active ingredients effective to act as an inhibitor of ENPP1 or effective to treat or prevent an infection (including but not limited to bacterial infections and viral infections), proliferative disorders (such as cancer or a tumor), cardiovascular disease (including cardiac injury), diabetes or insulin resistance, chondrocalcinosis, calcium pyrophosphate deposition disorder (pseudogout), or hypophosphatasia.

As used herein, the terms “modulate”, or “modulating” refer to the ability to increase or decrease the activity of one or more pyrophosphatase or phosphodiesterase. Accordingly, compounds of the invention can be used in methods of modulating a pyrophosphatase or phosphodiesterase by contacting the pyrophosphatase or phosphodiesterase with any one or more of the compounds or compositions described herein. In some embodiments, the compounds can act as inhibitors of one or more pyrophosphatase or phosphodiesterase. In some embodiments, the compounds can act to stimulate the activity of one or more pyrophosphatase or phosphodiesterase. In further embodiments, the compounds of the invention can be used to modulate activity of a pyrophosphatase or phosphodiesterase in an individual in need of modulation of the receptor by administering a modulating amount of a compound as described herein.

As used herein, the term “contacting” refers to the bringing together of indicated moieties in an in vitro system or an in vivo system. For example, “contacting” the ENPP1 enzyme with a compound of Formula (I) includes the administration of a compound of the present invention to an individual or patient, such as a human, having ENPP1, as well as, for example, introducing a compound of Formula (I) into a sample containing a cellular or purified preparation containing ENPP1 enzyme.

The term “ENPP1 inhibitor” or “ENPP1 modulator” refers to an agent capable of inhibiting the enzymatic activity of ectonucleotide pyrophosphatase/phosphodiesterase 1. The ENPP1 inhibitor may be a reversible or irreversible ENPP1 inhibitor. A “reversible ENPP1 inhibitor” is a compound that reversibly inhibits ENPP1 enzyme activity either at the catalytic site or at a non-catalytic site and “an irreversible ENPP1 inhibitor” is a compound that irreversibly destroys ENPP1 enzyme activity by forming a covalent bond with the enzyme.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

The compounds of Formula (I) can inhibit the activity of ENPP1. For example, the compounds of Formula (I) can be used to inhibit activity of ENPP1 in a cell or in an individual in need of modulation of ENPP1 by administering an inhibiting amount of a compound of Formula (I), or a salt thereof. In some embodiments, the compound of the present disclosure is cell permeable. In some embodiments, the compounds of Formula (I) inhibit the activity of ENPP1. In some embodiments, ENPP1 activity is inhibited by about 10%, about 20%, about 30%, about 40%, about 50%, about 60%, about 70%, about 80%, about 90%, or about 100%, including all ranges and values therebetween.

In some embodiments are methods of inhibiting ENPP1 comprising contacting ENPP1 with an effective amount of a compound of Formula (I), or a pharmaceutically acceptable salt, solvate, hydrate, isomer, tautomer, racemate, isotope, or pharmaceutical composition thereof.

The compounds of Formula (I) and pharmaceutical compositions comprising at least one compound of Formula (I) are useful in treating or preventing any disease or condition associated with ENPP1. In some embodiments, the invention provides a method of treating a patient suffering from or susceptible to a medical condition that is associated with ENPP1. A number of medical conditions can be treated. The method comprises administering to a subject in need thereof, an effective amount of a compound of Formula (I), or a pharmaceutically acceptable salt, solvate, hydrate, isomer, tautomer, racemate, isotope, or pharmaceutical composition thereof. In some embodiments the ENPP1 dependent condition is an infection (including but not limited to bacterial infections and viral infections), a proliferative disorder (such as cancer or a tumor), cardiovascular disease (including cardiac injury), diabetes or insulin resistance, chondrocalcinosis, calcium pyrophosphate deposition disorder (pseudogout), or hypophosphatasia.

In some embodiments, the compounds of Formula (I) and pharmaceutical compositions comprising at least one compound of Formula (I) are useful as vaccine boosters.

In some embodiments, the ENPP1 dependent condition is a disorder of uncontrolled cellular proliferation. In some embodiments, the disorder of uncontrolled proliferation is a cancer or a tumor. In some embodiments, the present disclosure provides a method of treating cancer or a tumor in a subject in need thereof comprising administering to the subject a therapeutic amount of a compound of Formula (I), or a pharmaceutically acceptable salt, solvate, hydrate, isomer, tautomer, racemate, isotope, or composition thereof, according to the present application.

In some embodiments, the cancer is a solid tumor. In some embodiments, the cancer is selected from adrenal, liver, kidney, bladder, breast, colon, gastric, ovarian, cervical, uterine, esophageal, colorectal, prostate, pancreatic, lung (both small cell and non-small cell), thyroid, carcinomas, sarcomas, glioblastomas, melanoma and various head and neck tumors. In some embodiments, the solid tumor is breast cancer, lung cancer, or glioblastoma.

In some embodiments of the present disclosure, the cancer is a hematologic malignancy. In some embodiments, the hematologic malignancy is a leukemia, a lymphoma, or a myeloma. In some embodiments, the hematologic malignancy is a B-cell malignancy. In certain embodiments, the hematologic malignancy is multiple myeloma.

In some embodiments of the present disclosure, the cancer is a relapsed or refractory cancer. In some embodiments of the present disclosure, the cancer is a metastatic cancer.

In some embodiments, the ENPP1 dependent condition is a bacterial infection. In some embodiments, the present disclosure provides a method of treating or preventing a bacterial infection in a subject in need thereof comprising administering to the subject a therapeutic amount of a compound of Formula (I), or a pharmaceutically acceptable salt, solvate, hydrate, isomer, tautomer, racemate, isotope, or composition thereof, according to the present application.

In some embodiments, the bacterial infection is a gram-positive infection. In other embodiments, the bacterial infection is a gram-negative infection. In some embodiments, the gram-positive infection is an infection caused by S. aureus (e.g., methicillin-susceptible or methicillin-resistant) or E. faecium. In other embodiments, the gram-negative infection is an infection caused by K. pneumoniae, P. aeruginosa, E. cloacae, or A. baumannii. In some embodiments, the bacterial infection is multidrug-resistant. In some embodiments, the bacterial infection is caused by M. tuberculosis. Accordingly, in various embodiments, the compounds and compositions of the present disclosure are effective in treating tuberculosis.

In some embodiments, the ENPP1 dependent condition is a viral infection. In some embodiments, the present disclosure provides a method of treating or preventing a viral infection in a subject in need thereof comprising administering to the subject a therapeutic amount of a compound of Formula (I), or a pharmaceutically acceptable salt, solvate, hydrate, isomer, tautomer, racemate, isotope, or composition thereof, according to the present application.

In some embodiments, the viral infection is due to a DNA virus. In some embodiments, the viral infection is a due to a herpesvirus. In certain embodiments, the herpesvirus is selected from herpes simplex viruses 1 (HSV-1), herpes simplex viruses 2 (HSV-2), varicella-zoster virus (VZV), Epstein-Barr virus (EBV), human cytomegalovirus (HCMV), human herpesvirus 6A (HHV-6A), human herpesvirus 6B (HHV-6B), human herpesvirus 7 (HHV-7), and Kaposi's sarcoma-associated herpesvirus (KSHV). In a specific embodiment, the herpesvirus is herpes simplex viruses 1 (HSV-1). In some embodiments of the present disclosure, the viral infection is a due to a retrovirus. In some embodiments, the retrovirus is human immunodeficiency virus (HIV). In some embodiments, the viral infection is a due to a hepatitis virus. In certain embodiments, the hepatitis virus is hepatitis B virus (HBV) or hepatitis D virus (HDV). In certain other embodiments, the viral infection is due to vaccinia virus (VACV), adenovirus, or human papillomaviruses (HPV). In some embodiments of the present disclosure, the viral infection is due to an RNA virus. In certain embodiments, the viral infection is due to dengue fever virus, yellow fever virus, Ebola virus, Marburg virus, Venezuelan encephalitis virus, or zika virus.

In some embodiments are provided uses of a compound of Formula (I), or a pharmaceutically acceptable salt, solvate, hydrate, isomer, tautomer, racemate, isotope, or pharmaceutical composition thereof in the manufacture of a medicament. In some embodiments the medicament is for the treatment of an infection (including but not limited to bacterial infections and viral infections), a proliferative disorder (such as cancer or a tumor), cardiovascular disease (including cardiac injury), diabetes or insulin resistance, chondrocalcinosis, calcium pyrophosphate deposition disorder (pseudogout) or hypophosphatasia

The present invention further provides methods of treating diseases associated with activity or expression, including abnormal activity and/or overexpression, of ENPP1 in an individual (e.g., patient) by administering to the individual in need of such treatment a therapeutically effective amount or dose of a compound of Formula (I) or a pharmaceutical composition thereof. Example diseases can include any disease, disorder or condition that is directly or indirectly linked to expression or activity of ENPP1 enzyme, such as over expression or abnormal activity. A ENPP1 associated disease can also include any disease, disorder or condition that can be prevented, ameliorated, or cured by modulating ENPP1 enzyme activity. Examples of ENPP1 associated diseases and disorders include an infection (including but not limited to bacterial infections and viral infections), a proliferative disorder (such as cancer or a tumor), cardiovascular disease (including cardiac injury), diabetes or insulin resistance, chondrocalcinosis, calcium pyrophosphate deposition disorder (pseudogout), or hypophosphatasia.

The compounds of Formula (I) and pharmaceutical compositions comprising at least one compound of Formula (I) may be administered to animals, preferably mammals (e.g., domesticated animals, cats, dogs, mice, rats), and more preferably humans. Any method of administration may be used to deliver the compound or pharmaceutical composition to the patient. In certain embodiments, the compound of Formula (I) or pharmaceutical composition comprising at least one compound of Formula (I) is administered orally. In other embodiments, the compound of Formula (I) or pharmaceutical composition comprising at least one compound of Formula (I) is administered parenterally.

Also described herein are uses of a compound of formula (I), or a pharmaceutically acceptable salt, solvate, hydrate, isomer, tautomer, racemate, isotope, or composition thereof, for inhibiting the activity of ENPP1.

Also described herein are uses of a compound of formula (I), or a pharmaceutically acceptable salt, solvate, hydrate, isomer, tautomer, racemate, isotope, or composition thereof, for treating a disease or disorder associated with the activity ENPP1. Examples of ENPP1 associated diseases or disorders include infection (including but not limited to bacterial infections and viral infections), proliferative disorders (such as cancer or a tumor), cardiovascular disease (including cardiac injury), diabetes or insulin resistance, chondrocalcinosis, calcium pyrophosphate deposition disorder (pseudogout), or hypophosphatasia

### Combination Therapy

One or more additional pharmaceutical agents or treatment methods such as, for example, anti-viral agents; chemotherapeutics, anti-oncology agents, or other anti-cancer agents; immune enhancers; immunosuppressants; radiation; anti-tumor and anti-viral vaccines; cytokine therapy (e.g., IL2 and GM-CSF); and/or tyrosine kinase inhibitors can be optionally used in combination with the compounds of Formula (I) for treatment of ENPP1 associated diseases, disorders, or conditions. The agents can be combined with the present compounds in a single dosage form, or the agents can be administered simultaneously or sequentially as separate dosage forms.

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral mutes, intravenous mutes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally, or all therapeutic agents may be administered by intravenous injection. Combination therapy also can embrace the administration of the therapeutic agents as described above in further combination with other biologically active ingredients and non-drug therapies (e.g., surgery or radiation treatment.) Where the combination therapy further comprises a non-drug treatment, the non-dmg treatment may be conducted at any suitable time so long as a beneficial effect from the co-action of the combination of the therapeutic agents and non-dmg treatment is achieved. For example, in appropriate cases, the beneficial effect is still achieved when the non-drug treatment is temporally removed from the administration of the therapeutic agents, perhaps by days or even weeks.

In some embodiments, the present invention provides a combined preparation of a compound of Formula (I), and/or a pharmaceutically acceptable salt thereof, a stereoisomer thereof or a tautomer thereof; and additional therapeutic agent(s) for simultaneous, separate, or sequential use in the treatment and/or prophylaxis of multiple ENPP1 associated diseases or disorders.

### Routes of Administration

The compounds of this invention can be administered for any of the uses described herein by any suitable means, for example, orally, such as tablets, capsules (each of which includes sustained release or timed release formulations), pills, powders, granules, elixirs, tinctures, suspensions (including nanosuspensions, micro suspensions, spray-dried dispersions), syrups, and emulsions; sublingually; buccally; parenterally, such as by subcutaneous, intravenous, intramuscular, or intratarsal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

### Kits

The present invention also includes pharmaceutical kits useful, for example, in the treatment or prevention of ENPP1 associated diseases or disorders, and other diseases referred to herein, which include one or more containers containing a pharmaceutical composition comprising a therapeutically effective amount of a compound of Formula (I), or a pharmaceutically acceptable salt, solvate, hydrate, isomer, tautomer, racemate, or isotope thereof. Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

### Compound Synthesis

Compounds having the structure of Formulas (I) can be synthesized using standard synthetic techniques known to those of skill in the art. For example, compounds of the present disclosure can be synthesized using the general synthetic procedures set forth in Schemes 1-5.

Modifications to these methods will be apparent to one skilled in the art. To this end, the reactions, processes, and synthetic methods described herein are not limited to the specific conditions described in the following experimental section, but rather are intended as a guide to one with suitable skill in this field. For example, reactions may be carried out in any suitable solvent, or other reagents to perform the transformation[s] necessary. Generally, suitable solvents are protic or aprotic solvents which are substantially non-reactive with the reactants, the intermediates, or products at the temperatures at which the reactions are carried out (i.e., temperatures which may range from the freezing to boiling temperatures). A given reaction may be carried out in one solvent or a mixture of more than one solvent. Depending on the particular reaction, suitable solvents for a particular work-up following the reaction may be employed.

Unless otherwise indicated, conventional methods of mass spectroscopy (MS), liquid chromatography-mass spectroscopy (LCMS), NMR, HPLC, protein chemistry, biochemistry, recombinant DNA techniques, and pharmacology are employed. Compounds are prepared using standard organic chemistry techniques such as those described in, for example, March's Advanced Organic Chemistry, 7^{th} Edition, John Wiley and Sons, Inc (2013). Alternate reaction conditions for the synthetic transformations described herein may be employed such as variation of solvent, reaction temperature, reaction time, as well as different chemical reagents and other reaction conditions. As necessary, the use of appropriate protecting groups may be required. The incorporation and cleavage of such groups may be carried out using standard methods described in Peter G. M. Wuts and Theodora W. Green, Protecting Groups in Organic Synthesis, 4^{th} Edition, Wiley-Interscience. (2006). All starting materials and reagents are commercially available or readily prepared.

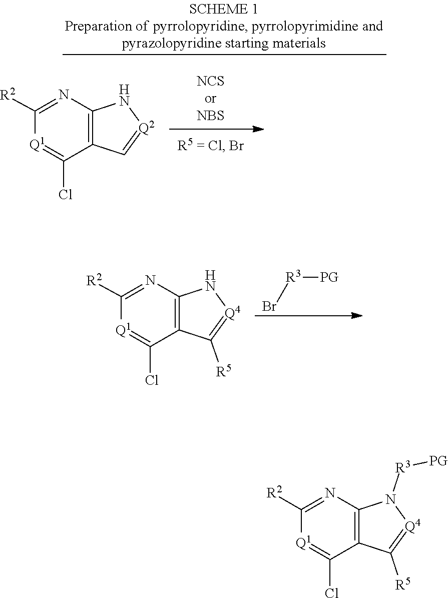

4-chloro-3-R⁵-1-R³(PG)-6-R²-1H-pyrrolo[2,3-b]pyridine; 4-chloro-5-R⁵-7-R³(PG)-2-R²-7H-pyrrolo[2,3-d]pyrimidine; 4-chloro-3-R⁵-1-R³(PG)-6-R²-1H-pyrazolo[3,4-b]pyridine; and 4-chloro-3-R⁵-1-R³(PG)-6-R²-1H-pyrazolo[3,4-d]pyrimidine may be prepared according to Scheme 1. Treatment of 4-chloro-2-R²-1H-pyrrolo[2,3-b]pyridine, 4-chloro-2-R²-7H-pyrrolo[2,3-d]pyrimidine, 4-chloro-6-R²-1H-pyrazolo[3,4-b]pyridine or 4-chloro-6-R²-1H-pyrazolo[3,4-d]pyrimidine with a halogenating agent, e.g. NCS or NBS introduces R⁵ as a halogen. Optionally protected R³ is introduced as shown.

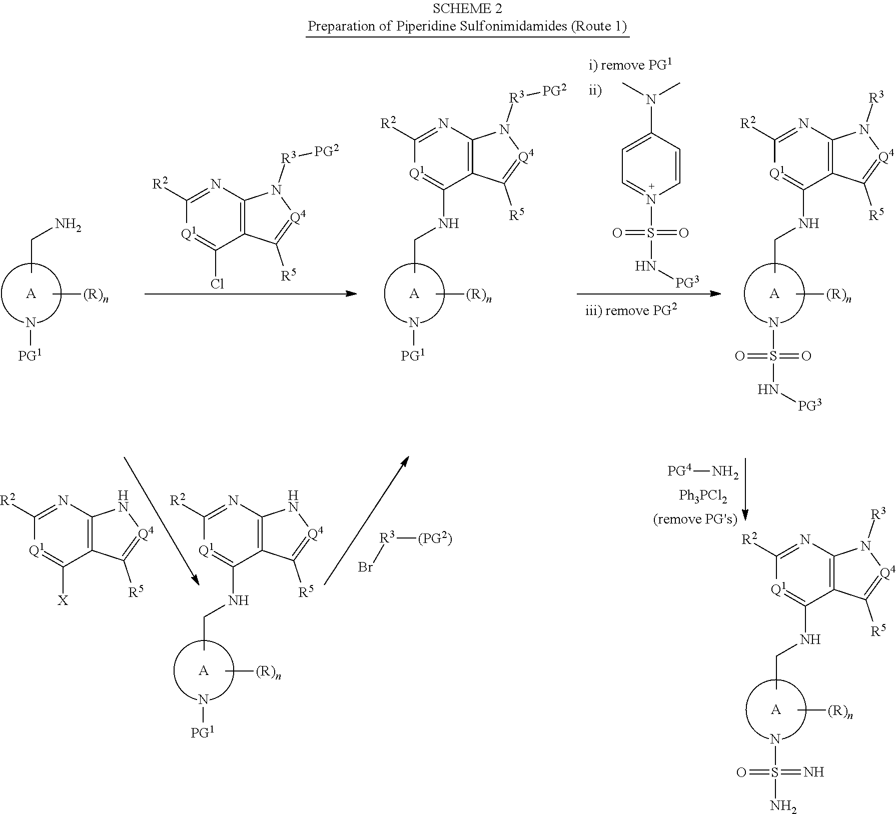

As shown in Scheme 2, cyclic amine-containing methanamine derivatives are coupled with pyrrolopyridine, pyrrolopyrimidine and pyrazolopyridine derivatives, using protecting groups as required. The piperidine amine is deprotected and subsequently converted to the sulfonyl urea which is then activated via chlorination and then reacted with a suitably protected amine. Removal of all protecting groups provides the desired piperidine sulfonimidamides, as shown in Scheme 2.

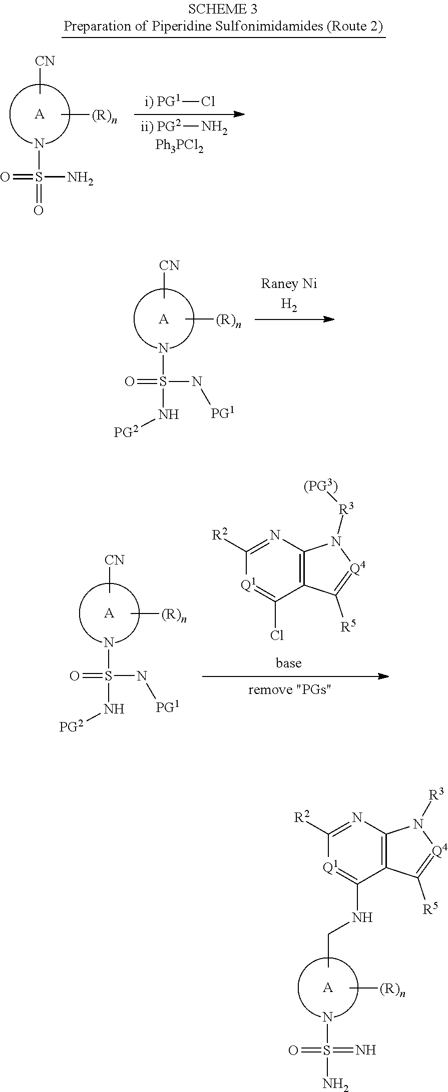

In an alternate synthetic route, nitrile-containing cyclic amine-1-sulfonamide derivatives are converted to the sulfonimidamides, using protecting groups as necessary. Reduction of the nitrile group to the amine, followed by coupling with pyrrolopyridine, pyrrolopyrimidine and pyrazolopyridine derivatives and subsequent removal of protecting groups, as required, provides the desired final compounds, as shown in Scheme 3.

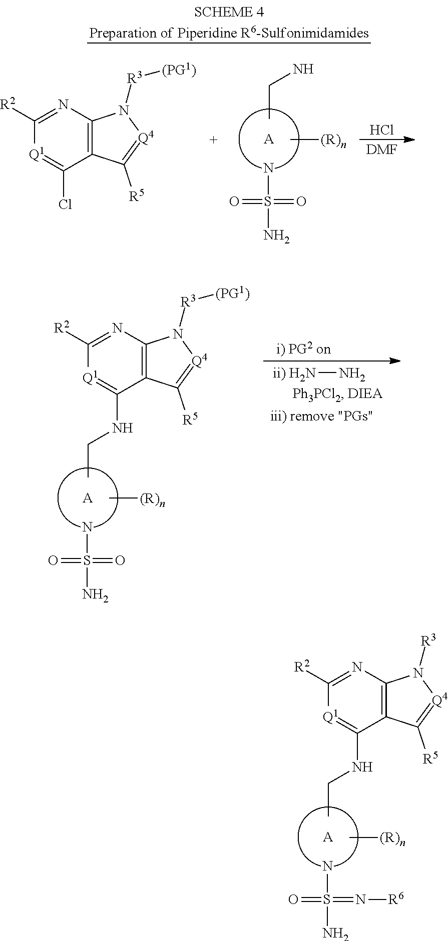

Cyclic amine-sulfonyl urea derivatives are coupled with pyrrolopyridine, pyrrolopyrimidine and pyrazolopyridine derivatives, using protecting groups as required. The sulfonylurea amine is protected, as required, and then activated via chlorination (for example) then reacted with an R⁶ substituted amine to form the R⁶ substituted sulfonimidamide. Removal of any remaining protecting groups provides the desired compounds, as shown in Scheme 4. The final racemic product may be separated into pure enantiomers using techniques typical for chiral separation.

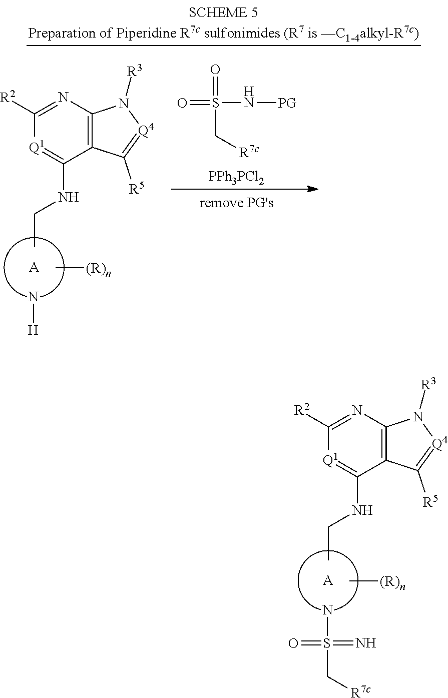

R^{7c} containing sulfonamide is activated (via chlorination) via treatment with PPh₃Cl₂, for example, and subsequently reacted with pyrrolopyridine, pyrrolopyrimidine or pyrazolopyridine are-substituted cyclic amines, which after removal of protecting groups as required, provides the desired sulfonimides, as shown in Scheme 5. The final racemic product may be separated into pure enantiomers using techniques typical for chiral separation.

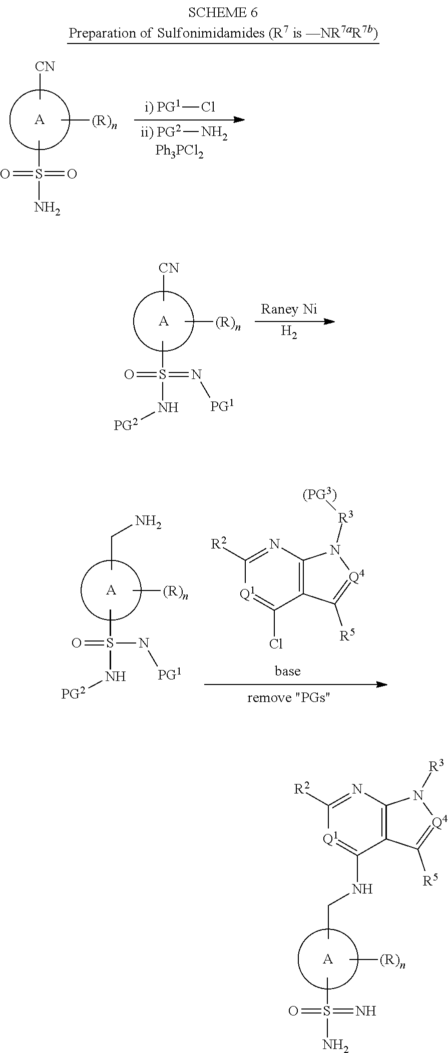

The sulfonamide group of cyclic nitrile-containing sulfonamide derivatives is protected with an appropriate protecting group. The protected sulfonamide is then converted to a sulfonimidamide by treatment with a chlorinating (activating) agent such as Ph₃PCl₂ and then reacted with a suitable amine, using protecting groups as required. The nitrile group is then reduced to the methanamine which is then coupled with pyrrolopyridine, pyrrolopyrimidine and pyrazolopyridine derivatives. Removal of all protecting groups provides the desired sulfonimidamides, as shown in Scheme 6.

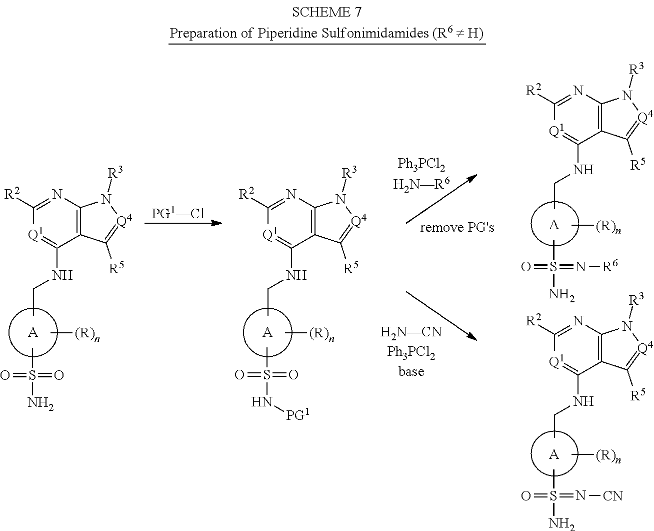

Protected sulfonamides are first chlorinated (activated) using a reagent such as Ph₃PCl₂, then subsequently reacted with R⁶ amines to provide R⁶ substituted sulfonimidamides, then globally deprotected to afford final substituted sulfonimidamides, as shown in Scheme 7. The final racemic product may be separated into pure enantiomers using techniques typical for chiral separation.

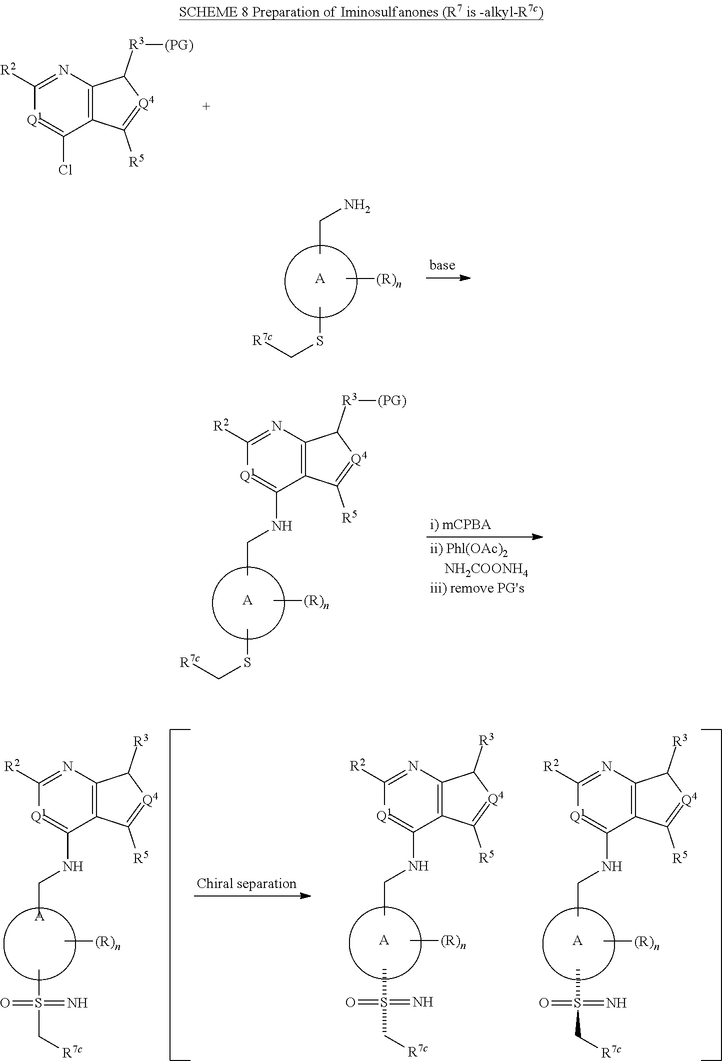

R^{7c} substituted sulfide cyclic methanamines are coupled with pyrrolopyridine, pyrrolopyrimidine and pyrazolopyridine derivatives. The sulfide group is oxidized to the sulfoxide, which is then converted to the iminosulfanone via treatment with, for example, PhI(Oac)₂ and NH₂CO₂NH₄, employing protecting groups as required, as shown in Scheme 8. The final product may be separated into pure enantiomers using techniques typical for chiral separation.

### EXAMPLES

The following examples are provided for illustrative purposes only and not to limit the scope of the claims provided herein. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1

### Synthesis of 4-(((1-ethyl-1H-pyrazolo[3,4-b]pyridin-4-yl)amino)methyl)piperidine-1-sulfonimidamide (Compound 1A)

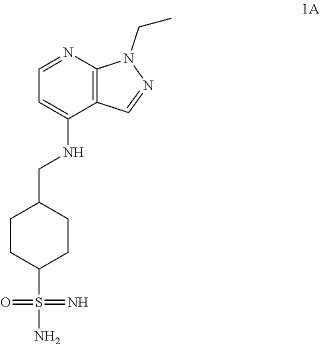 [SMILES: CCn1ncc2c(NCC3CCC(S(=N)(N)=O)CC3)ccnc21]

### Step 1: tert-butyl 4-(((1-ethyl-1H-pyrazolo[3,4-b]pyridine-4-yl)amino)methyl) piperidine-1-carboxylate

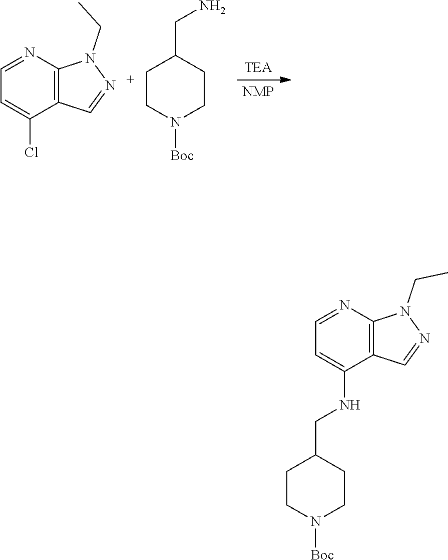

To a solution of 4-chloro-1-ethyl-1H-pyrazolo[3,4-b]pyridine (3.0 g, 16.52 mmol) and tert-butyl 4-(aminomethyl)piperidine-1-carboxylate (8.85 g, 41.29 mmol) in NMP (50 mL) was added triethylamine (10.03 g, 99.11 mmol). The reaction mixture was stirred at 130° C. for 16 h and cooled to room temperature. The cooled reaction mixture was quenched with water (100 mL) and then extracted with ethyl acetate (150 mL×3). The organic layers were combined, washed with brine (200 mL), dried over sodium sulfate, filtered, and concentrated under vacuum. The residue was purified by column chromatography on silica gel eluting with ethyl acetate in petroleum ether (57%) to give tert-butyl 4-(((1-ethyl-1H-pyrazolo[3,4-b]pyridine-4-yl)amino)methyl)piperidine-1-carboxylate (5.0 g, 82%) as a yellow oil. LCMS (ESI) m/z=360.15 (M+H).

### Step 2: 1-ethyl-N-(piperidin-4-ylmethyl)-1H-pyrazolo[3,4-b]pyridine-4-amine hydrochloride

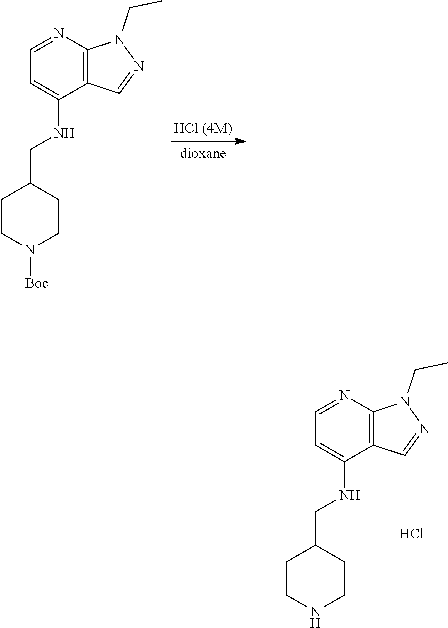 [SMILES: CCn1ncc2c(NCC3CCN(C(=O)OC(C)(C)C)CC3)ccnc21 ; CCn1ncc2c(NCC3CCNCC3)ccnc21 ; Cl]

A mixture of tert-butyl 4-(((1-ethyl-1H-pyrazolo[3,4-b]pyridine-4-yl)amino) methyl)piperidine-1-carboxylate (5.0 g, 13.91 mmol) and hydrogen chloride (4 M in dioxane, 80 mL) was stirred at 20° C. for 2 h. The resulting mixture was concentrated under vacuum to give 1-ethyl-N-(piperidin-4-ylmethyl)-1H-pyrazolo[3,4-b]pyridine-4-amine hydrochloride (7.0 g, crude) as a yellow solid. LCMS (ESI) m/z=260.10 (M+H).

### Step 3: tert-butyl ((4-(((1-ethyl-1H-pyrazolo[3,4-b]pyridine-4-yl)amino)methyl) piperidin-1-yl)sulfonyl)carbamate

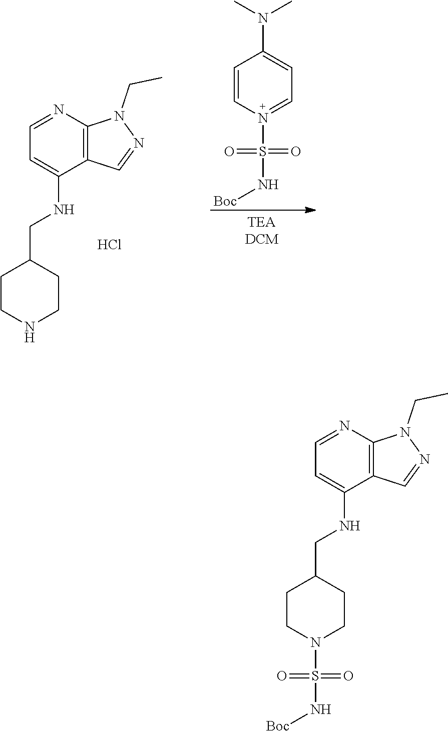

To a solution of 1-ethyl-N-(piperidin-4-ylmethyl)-1H-pyrazolo[3,4-b]pyridine-4-amine hydrochloride (1.0 g, crude) and 1-(N-(tert-butoxycarbonyl)sulfamoyl)-4-(dimethylamino) pyridine-1-ium (767 mg, 2.54 mmol) in DCM (20 mL) was added triethylamine (2.05 g, 20.28 mmol). The reaction mixture was stirred at 25° C. for 2 h, quenched with water (30 mL), and then extracted with DCM (80 mL×3). The organic layers were combined, washed with brine (100 mL), dried over sodium sulfate, filtered, and concentrated under vacuum. The residue was purified by column chromatography (silica gel, methanol:dichloromethane=6%: 94%) to give tert-butyl ((4-(((1-ethyl-1H-pyrazolo[3,4-b]pyridine-4-yl)amino)methyl)piperidin-1-yl)sulfonyl)carbamate (450 mg, 30%) as a yellow solid. LCMS (ESI) m/z=439.15 (M+H).

### Step 4: 4-(((1-ethyl-1H-pyrazolo[3,4-b]pyridine-4-yl)amino)methyl)piperidine-1-sulfonamide hydrochloride

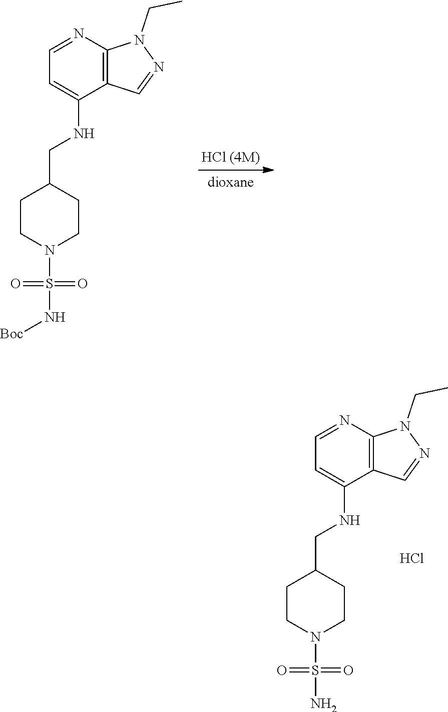 [SMILES: CCn1ncc2c(NCC3CCN(S(=O)(=O)NC(=O)OC(C)(C)C)CC3)ccnc21 ; CCn1ncc2c(NCC3CCN(S(N)(=O)=O)CC3)ccnc21 ; Cl]

A mixture of tert-butyl ((4-(((1-ethyl-1H-pyrazolo[3,4-b]pyridine-4-yl)amino) methyl)piperidin-1-yl)sulfonyl)carbamate (450 mg, 1.03 mmol) and hydrogen chloride (4 M in dioxane, 10 mL) was stirred at 20° C. for 2 h. Then the resulted mixture was concentrated under vacuum to give 4-(((1-ethyl-1H-pyrazolo[3,4-b]pyridine-4-yl)amino)methyl)piperidine-1-sulfonamide hydrochloride (500 mg, crude) as a yellow solid. LCMS (ESI) m/z=339.00 (M+H).

### Step 5: N-(tert-butyldimethylsilyl)-4-(((1-ethyl-1H-pyrazolo[3,4-b]pyridine-4-yl)amino) methyl)piperidine-1-sulfonamide

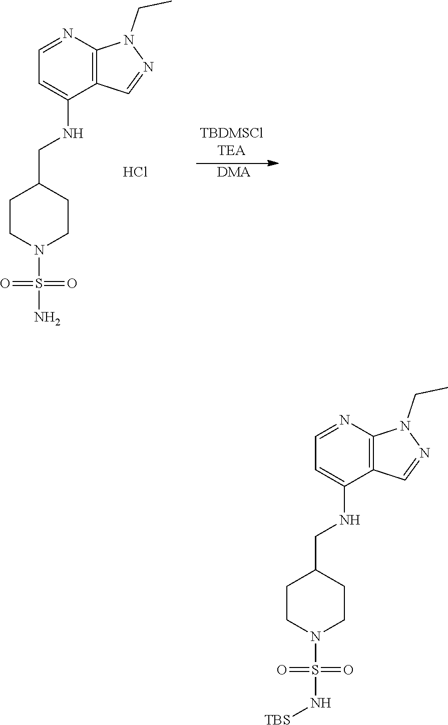

To a solution of 4-(((1-ethyl-1H-pyrazolo[3,4-b]pyridine-4-yl)amino)methyl) piperidine-1-sulfonamide hydrochloride (450 mg, crude) and TBDMSCl (271 mg, 1.80 mmol) in DMA (5 mL) was added triethylamine (486 mg, 4.80 mmol). The reaction mixture was stirred under N₂ at 20° C. for 16 h, quenched with water (20 mL), and then extracted with ethyl acetate (60 mL×3). The organic layers were combined, washed with brine (80 mL), dried over sodium sulfate, filtered, and concentrated under vacuum. The residue was purified by column chromatography to give N-(tert-butyldimethylsilyl)-4-(((1-ethyl-1H-pyrazolo[3,4-b]pyridine-4-yl)amino)methyl)piperidine-1-sulfonamide (330 mg, 57%) as a yellow solid. LCMS (ESI) m/z=453.15 (M+H).

### Step 6: N′-(tert-butyldimethylsilyl)-4-(((1-ethyl-1H-pyrazolo[3,4-b]pyridine-4-yl)amino)methyl)-N-(4-methoxybenzyl)piperidine-1-sulfonimidamide

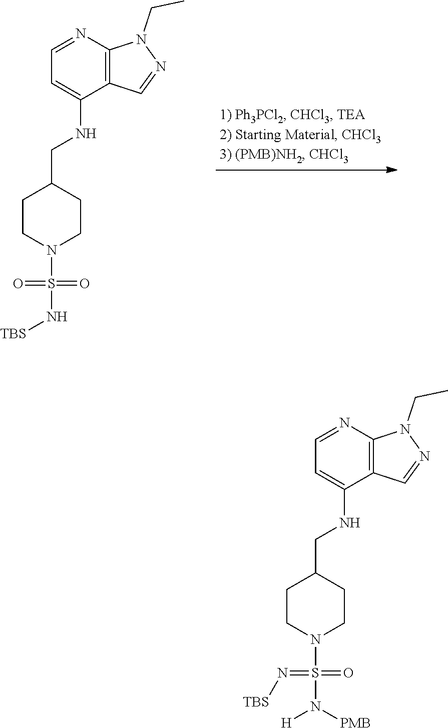

To a mixture of PPh₃Cl₂ (920 mg, 2.76 mmol) in CHCl₃ (10 mL) was added triethylamine (559 mg, 5.52 mmol) under N₂. The reaction mixture was stirred at 0° C. for 15 min. N-(tert-butyldimethylsilyl)-4-(((1-ethyl-1H-pyrazolo[3,4-b]pyridine-4-yl)amino)methyl) piperidine-1-sulfonamide (250 mg, 0.55 mmol) was added. After stirring at 0° C. for 30 min, PMBNH₂ (758 mg, 5.52 mmol) was added. The reaction mixture was allowed to stir for another 30 min at 0° C., and then 30 min at 20° C. The reaction mixture was quenched with water (30 mL) and extracted with ethyl acetate (60 mL×3). The organic layers were combined, washed with brine (80 mL), dried over sodium sulfate, filtered, and concentrated under vacuum. The residue was purified by column chromatography to give N′-(tert-butyldimethylsilyl)-4-(((1-ethyl-1H-pyrazolo[3,4-b]pyridine-4-yl)amino)methyl)-N-(4-methoxybenzyl)piperidine-1-sulfonimidamide (200 mg, crude) as a yellow oil. LCMS (ESI) m/z=572.30 (M+H).

### Step 7: 4-(((1-ethyl-1H-pyrazolo[3,4-b]pyridine-4-yl)amino)methyl)piperidine-1-sulfonimidamide

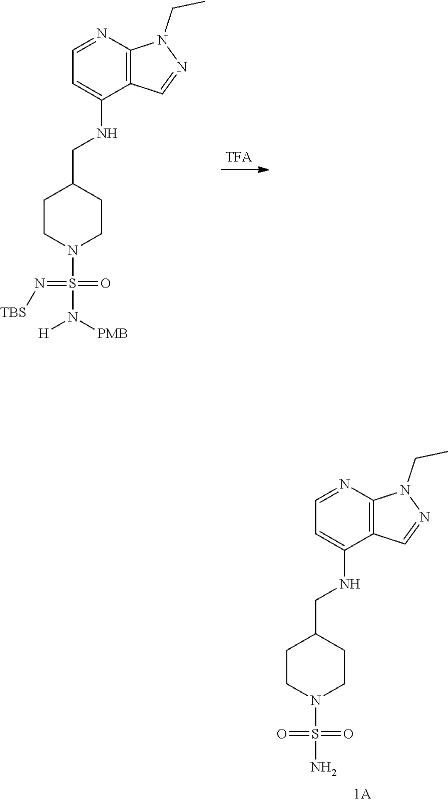

A solution of N′-(tert-butyldimethylsilyl)-4-(((1-ethyl-1H-pyrazolo[3,4-b]pyridine-4-yl)amino)methyl)-N-(4-methoxybenzyl)piperidine-1-sulfonimidamide (170 mg, crude) in TFA (10 mL) was stirred at 75° C. for 16 h. The cooled reaction mixture was concentrated under vacuum. The residue was purified by Prep-HPLC (Column: Xbridge Prep C18 OBD Column, 19×150 mm, 5 μm; Mobile Phase A: Water (0.05% TFA), Mobile Phase B: I; Flow rate: 25 mL/min; Gradient: 5% B to 25% B in 7 min, 25% B; Wave Length: 254 nm; RT (min): 6.0 min) to give 4-(((1-ethyl-1H-pyrazolo[3,4-b]pyridinedin-4-yl)amino)methyl)piperidine-1-sulfonimidamide (Compound 1A) (as TFA salt, 58.6 mg, 42%) as an off-white solid. LCMS (ESI) m/z=338.10 (M+H); ¹H NMR (300 MHz, DMSO-d₆) δ 8.87 (s, 2H), 8.38 (s, 1H), 8.14 (d, J=6.6 Hz, 1H), 6.55 (d, J=6.7 Hz, 1H), 4.40 (q, J=7.2 Hz, 2H), 3.71 (d, J=12.1 Hz, 2H), 3.40 (s, 2H), 2.84 (t, J=12.0 Hz, 2H), 1.99-1.78 (m, 3H), 1.44-1.25 (m, 5H).

### Synthesis of 4-(((3-phenyl-1H-pyrazolo[3,4-b]pyridinedin-4-yl)amino)methyl) benzenesulfonimidamide (Compound 1B)

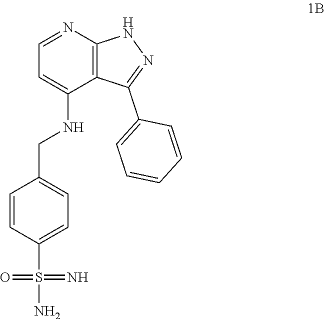 [SMILES: N=S(N)(=O)c1ccc(CNc2ccnc3[nH]nc(-c4ccccc4)c23)cc1]

Compound 1B was prepared according to similar procedures as described herein, particularly as described for Compound 1A.

¹H NMR (400 MHz, DMSO-d6) δ 8.29 (d, J=6.8 Hz, 1H), 8.02 (d, J=8.1 Hz, 2H), 7.82-7.58 (m, 8H), 6.42 (d, J=6.9 Hz, 1H), 4.84 (d, J=6.1 Hz, 2H); LCMS (ESI) m/z=379.15 (M+H).

### Synthesis of 4-(((3-chloro-1-ethyl-1H-pyrazolo[3,4-b]pyridine-4-yl)amino)methyl)piperidine-1-sulfonimidamide (Compound 1C)

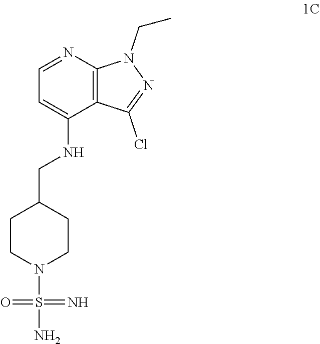 [SMILES: CCn1nc(Cl)c2c(NCC3CCN(S(=N)(N)=O)CC3)ccnc21]

Compound 1C was prepared according to similar procedures as described herein, particularly as described for Compound 1A.

¹H NMR (400 MHz, METHANOL-d4) δ ppm 8.14 (br d, J=7.25 Hz, 1H) 6.79-6.90 (m, 1H) 4.34-4.46 (m, 2H) 3.90 (br d, J=12.26 Hz, 2H) 3.60 (br d, J=6.00 Hz, 2H) 2.97 (br t, J=11.38 Hz, 2H) 1.92-2.09 (m, 3H) 1.44-1.58 (m, 5H). LCMS (ESI) m/z=372.1 (M+H).

### Example 2

### Synthesis of 4-(((1-ethyl-1H-pyrazolo[3,4-b]pyridine-4-yl)amino)methyl)benzene sulfonimidamide (Compound 2A)

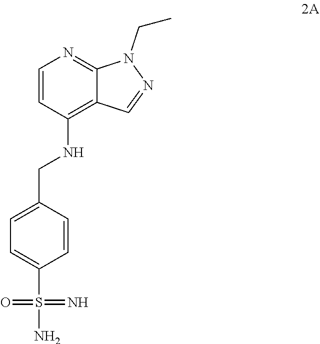 [SMILES: CCn1ncc2c(NCc3ccc(S(=N)(N)=O)cc3)ccnc21]

### Step 1: N-(tert-butyldimethylsilyl)-4-cyanobenzenesulfonamide

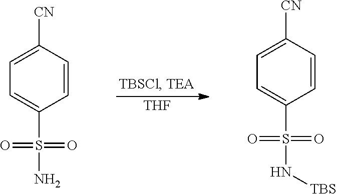

To a stirred mixture of 4-cyanobenzenesulfonamide (3.00 g, 16.47 mmol) and TEA (6.9 mL, 49.40 mmol) in THE (50 mL) was added TBSCl (3.10 g, 20.58 mmol) at 0° C. The reaction mixture was stirred for 16 h at 20° C. The reaction mixture was then filtered, and the filtrate was concentrated under vacuum. The residue was purified by column chromatography (silica gel, ethyl acetate:petroleum ether=0%: 100% to 30%: 70%) to give N-(tert-butyldimethylsilyl)-4-cyanobenzenesulfonamide (3.5 g, 71%) as a white solid. LCMS (ESI) m/z=295.00 (M+H).

### Step 2: N′-(tert-butyldimethylsilyl)-4-cyano-N-(4-methoxybenzyl)benzene sulfonimidamide

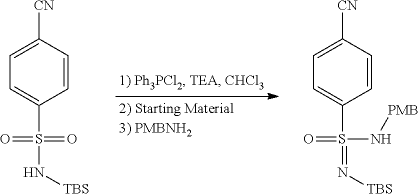

To a stirred suspension of Ph₃PCl₂ (6.31 g, 18.92 mmol) in dry CHCl₃ (70 mL) under N₂ atmosphere was added TEA (5.63 g, 55.66 mmol). The mixture was stirred for 30 min at room temperature, and a white suspension formed. The reaction mixture was cooled to 0° C. and a solution of N-(tert-butyldimethylsilyl)-4-cyanobenzenesulfonamide (3.3 g, 11.13 mmol) in dry CHCl₃ (30 mL) was added. The reaction mixture was stirred for 1 h at 0° C. To the reaction mixture was added PMBNH₂ (6.11 g, 44.53 mmol) in one portion. The mixture was stirred at 0° C. for 30 min then warmed to room temperature and stirred for 16 h. After completion, the mixture was filtered, and the filtrate was concentrated under vacuum. The residue was purified by column chromatography (silica gel, ethyl acetate:petroleum ether=0%: 100% to 30%: 70%) to afford N′-(tert-butyldimethylsilyl)-4-cyano-N-(4-methoxybenzyl)benzene sulfonimidamide (4.0 g, 86%) as a light-yellow solid. LCMS (ESI) m/z=416.05 (M+H).

### Step 3: 4-(aminomethyl′-N′-(tert-butyldimethylsilyl)-N-(4-methoxybenzyl) benzenesulfonimidamide

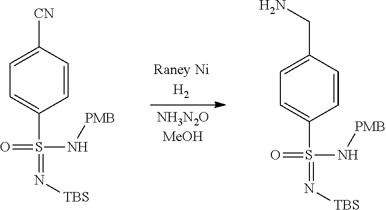

A mixture of N′-tert-butyldimethylsilyl-4-cyano-N-(4-methoxybenzyl)benzene sulfonimidamide (4.0 g, 9.62 mmol) and Raney Ni (50% slurry in water) (4.0 g) in NH₃·H₂O (10 mL, 30%) and methanol (40 mL) was stirred under hydrogen (2 atm) at room temperature for 2 h. The reaction mixture was filtered. The filtrate was concentrated under vacuum to give 4-(aminomethyl′-N′-(tert-butyldimethylsilyl)-N-(4-methoxybenzyl)benzenesulfonimidamide (3.5 g, crude) as a yellow solid. LCMS (ESI) m/z=420.10 (M+H).

### Step 4: N′-(tert-butyldimethylsilyl)-4-(((6-chloro-1-ethyl-1H-pyrazolo[3,4-b]pyridine-4-yl)amino)methyl)-N-(4-methoxybenzyl)benzenesulfonimidamide

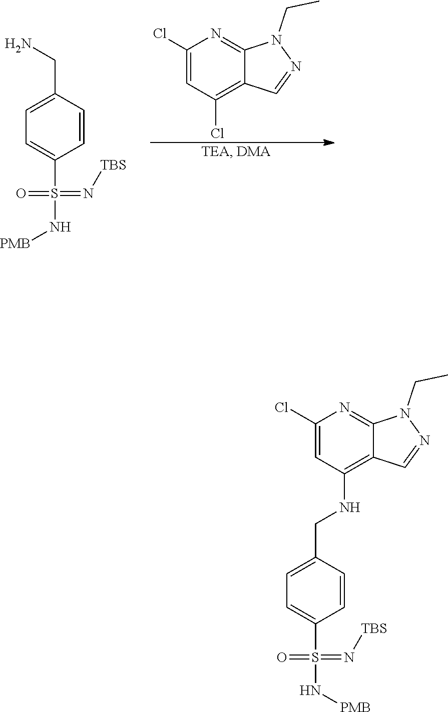

To a stirred mixture of 4,6-dichloro-1-ethyl-1H-pyrazolo[3,4-b]pyridine (250 mg, 1.16 mmol) and 4-(aminomethyl′-N′-(tert-butyldimethylsilyl)-N-(4-methoxybenzyl) benzenesulfonimidamide (728 mg, 1.74 mmol) in DMA (10 mL) was added triethylamine (0.5 mL, 3.58 mmol). The reaction mixture was stirred for 16 h at 80° C. and then cooled to room temperature. The cooled reaction mixture was quenched by the addition of water (30 mL) and extracted with ethyl acetate (30 mL×3). The combined organic layers were washed with brine (40 mL), dried over anhydrous Na₂SO₄, and concentrated under vacuum. The residue was purified by column chromatography (silica gel, methanol:dichloromethane=0%: 100% to 4%: 96%) to afford N′-(tert-butyldimethylsilyl)-4-(((6-chloro-1-ethyl-1H-pyrazolo[3,4-b]pyridine-4-yl)amino) methyl)-N-(4-methoxybenzyl)benzenesulfonimidamide (350 mg, 50%) as a yellow solid. LCMS (ESI) m/z=599.15 (M+H).

### Step 5: 4-(((1-ethyl-1H-pyrazolo[3,4-b]pyridine-4-yl)amino)methyl)-N-(4-methoxybenzyl)benzenesulfonimidamide

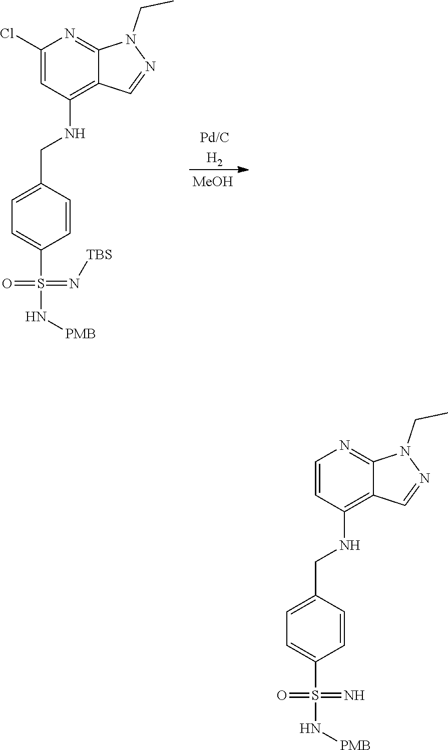

A mixture of N′-(tert-butyldimethylsilyl)-4-(((6-chloro-1-ethyl-1H-pyrazolo [3,4-b]pyridinedin-4-yl)amino)methyl)-N-(4-methoxybenzyl)benzenesulfonimidamide (350 mg, 0.58 mmol) and 10% palladium on carbon (300 mg) in methanol (15 mL) was stirred under hydrogen (2 atm) for 16 h at 25° C. The mixture was filtered, and the filtrate was concentrated under vacuum. The residue was purified by column chromatography (silica gel, methanol:dichloromethane=0%: 100% to 7%: 93%) to give 4-(((1-ethyl-1H-pyrazolo[3,4-b]pyridine-4-yl)amino)methyl)-N-(4-methoxybenzyl)benzenesulfonimidamide (80 mg, 30%) as a yellow solid. LCMS (ESI) m/z=451.10 (M+H).

### Step 6: 4-(((1-ethyl-1H-pyrazolo[3,4-b]pyridine-4-yl)amino)methyl) benzenesulfonimidamide

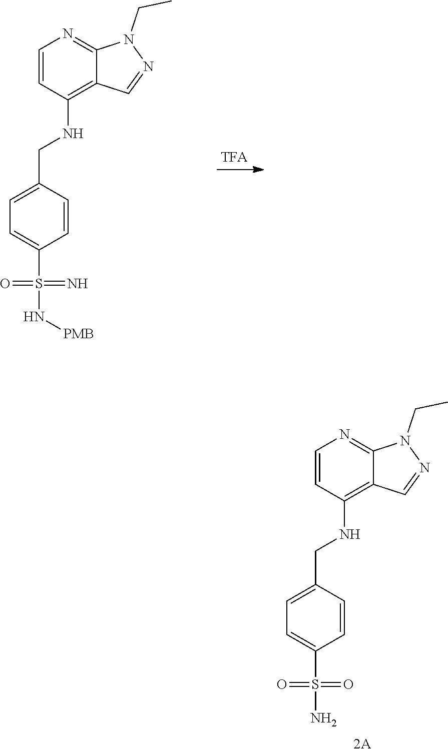

A mixture of 4-(((1-ethyl-1H-pyrazolo[3,4-b]pyridine-4-yl)amino)methyl)-N-(4-methoxybenzyl)benzenesulfonimidamide (80 mg, 0.18 mmol) in TFA (5 mL) was stirred at 80° C. for 16 h. After completion, the volatiles were removed under reduced pressure. The residue was purified by Prep-HPLC (Column: Xbridge Prep OBD C18 Column, 30×150 mm, 5 m; Mobile Phase A: Water (0.05% TFA), Mobile Phase IACN; Flow rate: 60 mL/min; Gradient: 5% B to 15% B in 7 min, 15% B; Wave Length: 254 nm; RT(min): 6.0) to afford 4-(((1-ethyl-1H-pyrazolo [3,4-b]pyridine-4-yl)amino)methyl)benzenesulfonimidamide (Compound 2A) (17 mg, 20%, as TFA salt) as a white solid. LCMS (ESI) m/z=331.05 (M+H); ¹H NMR (300 MHz, DMSO-d₆) δ 9.42 (s, 1H), 8.36 (s, 1H), 8.14 (d, J=6.6 Hz, 1H), 8.01 (d, J=8.4 Hz, 2H), 7.71 (d, J=8.4 Hz, 2H), 6.43 (d, J=6.3 Hz, 1H), 4.87 (d, J=5.4 Hz, 2H), 4.40 (q, J=7.2 Hz, 2H), 1.38 (t, J=7.2 Hz, 3H). ¹⁹F NMR (300 MHz, DMSO-d₆) δ −73.95.

### Synthesis of Compounds 2B-2D

Compounds 2B, 2C and 2D, listed below, were prepared according to similar procedures as described herein, particularly as described for compound 2A (m/z means m/z (ES+)(M+H)⁺).

TABLE 2

| Cmpd. | | | | |
|---|---|---|---|---|
| No. | Structure | Name | ¹H NMR | m/z |
| 2B | 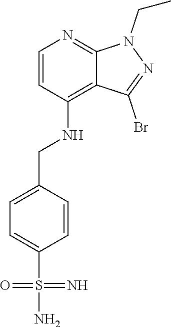 [SMILES: CCn1nc(Br)c2c(NCc3ccc(S(=N)(N)=O)cc3)ccnc21] | 4-(((3-bromo-1-ethyl-1H- pyrazolo[3,4-b]pyridine-4-y1) amino)methyl) benzenesulfonimidamide | (400 MHz, DMSO-d₆) δ 9.49 (brs, 3H), 8.05-8.01 (m, 3H), 7.75 (d, J = 8.3 Hz, 2H), 7.46-7.42 (m, 1H), 6.11 (dd, J = 5.7, 1.8 Hz, 1H), 4.80 (d, J = 6.2 Hz, 2H), 4.34 (q, J = 7.2 Hz, 2H), 1.37 (t, J = 7.2 Hz, 3H) | 410.95, 412.95 |
| 2C | 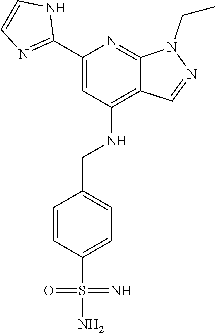 [SMILES: CCn1ncc2c(NCc3ccc(S(=N)(N)=O)cc3)cc(-c3ncc[nH]3)nc21] | 4-(((1-ethyl-6-(1H-imidazol-2-y1)- 1H-pyrazolo[3,4-b]pyridine-4-y1) amino)methyl) benzenesulfonimidamide | (300 MHz, DMSO-d₆) δ 8.62 (t, J = 6.0 Hz,1H), 8.28 (s, 1H), 8.00 (d, J = 8.7 Hz, 2H), 7.79-7.71 (m, 4H), 7.07 (s, 1H), 4.76 (d, J = 6.3 Hz, 2H), 4.48 (q, J = 7.2 Hz, 2H), 1.43 (t, J = 7.2 Hz, 3H) | 397.1 |
| 2D | 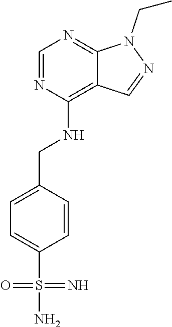 [SMILES: CCn1ncc2c(NCc3ccc(S(=N)(N)=O)cc3)ncnc21] | 4-(((1-ethyl-1H-pyrazolo[3,4-d] pyrimidin-4-y1)amino)methy1) benzenesulfonimidamide | (400 MHz, Methanol-d₄) δ 8.50- 8.39 (m, 2H), 8.23-8.11 (m, 2H), 7.92-7.81 (m, 2H), 5.08 (s, 2H), 4.50 (q, J = 7.2 Hz, 2H), 1.50 (t, J = 7.2 Hz, 3H) | 331.95 |

### Example 3

### Synthesis of 4-(((1-ethyl-1H-pyrazolo[3,4-b]pyridine-4-yl)amino)methyl)-N′-methylbenzenesulfonimidamide (Compound 3A)

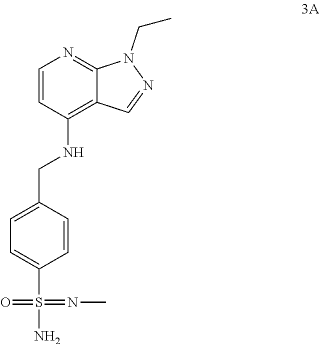 [SMILES: CCn1ncc2c(NCc3ccc(S(N)(=O)=NC)cc3)ccnc21]

### Step 1: N-(tert-butyldimethylsilyl)-4-(((1-ethyl-1H-pyrazolo[3,4-b]pyridine-4-yl)amino) methyl)benzenesulfonamide

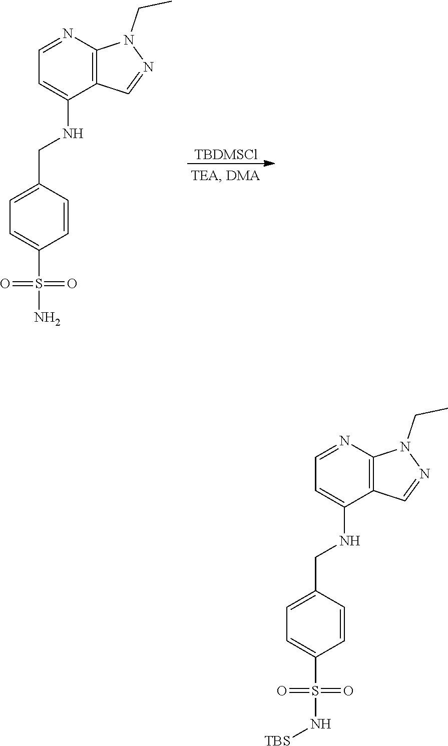

To a mixture of 4-(((1-ethyl-1H-pyrazolo[3,4-b]pyridine-4-yl)amino)methyl) benzenesulfonamide (500 mg, 1.51 mmol) and triethylamine (336 mg, 3.32 mmol) in DMA (15 mL) was added a solution of tert-butyldimethylsilyl chloride (284 mg, 1.89 mmol, 0.35 mL) in DMA (5 mL) at 0° C. under nitrogen atmosphere. The reaction mixture was stirred at 25° C. for 15 hours, quenched with water, and extracted with ethyl acetate (100 mL×3). The combined organic layers were washed with brine (100 mL×3) and dried over anhydrous sodium sulfate. The solid was filtered off and the filtrate was concentrated under reduced pressure to give N-(tert-butyldimethylsilyl)-4-(((1-ethyl-1H-pyrazolo[3,4-b]pyridine-4-yl)amino)methyl) benzenesulfonamide (600 mg, 79%) as a light-yellow solid. LCMS (ESI) m/z=891.3 (2M+H).

### Step 2: N′-(tert-butyldimethylsilyl)-4-(((1-ethyl-1H-pyrazolo[3,4-b]pyridine-4-yl) amino)methyl)-N-methylbenzenesulfonimidamide

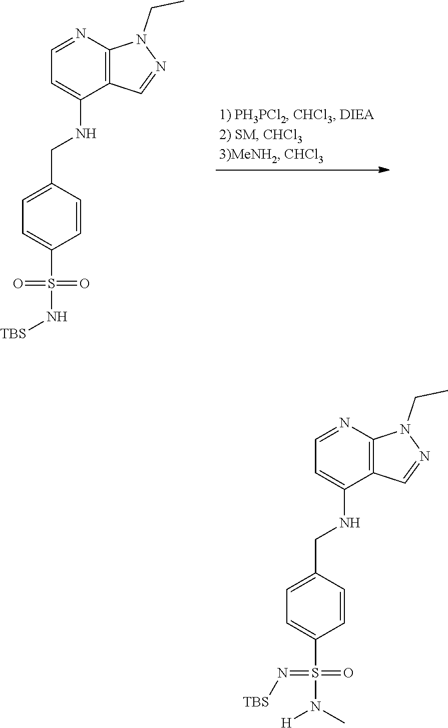

To a solution of triphenyldichlorophosphorane (1.12 g, 3.37 mmol) in chloroform (8 mL) was added N,N-diisopropylethylamine (1.6 g, 7.85 mmol) at 0° C. under nitrogen atmosphere. The reaction mixture was stirred at 25° C. for 10 minutes, followed by an addition of a solution of N-(tert-butyldimethylsilyl)-4-(((1-ethyl-1H-pyrazolo[3,4-b]pyridine-4-yl)amino) methyl) benzenesulfonamide (500 mg, 1.12 mmol) in chloroform (8 mL) at 0° C. The reaction mixture was then stirred at 0° C. for another 20 minutes, and a solution of methylamine (3.36 mL, 6.72 mmol, 2M in THF) in chloroform (5 mL) was added at 0° C. The resulted mixture was continued to stir for 30 minutes at 0° C. and 30 minutes at room temperature before it was filtered. The filtrate was concentrated under reduced pressure to give a residue, which was re-dissolved in ethyl acetate (100 mL), washed with brine (100 mL), dried over anhydrous sodium sulfate, and filtered. The filtrate was concentrated under reduced pressure to give a crude product, which was purified By column chromatography (silica gel, ethyl acetate:petroleum ether=0%: 100% to 50%: 50%) to afford N′-(tert-butyldimethylsilyl)-4-(((1-ethyl-1H-pyrazolo[3,4-b]pyridine-4-yl)amino)methyl)-N-methylbenzenesulfonimidamide (300 mg, 48%) as a yellow solid. LCMS (ESI) m/z=428.20 (M-CH₃NH).

### Step 3: 4-(((1-ethyl-1H-pyrazolo[3,4-b]pyridine-4-yl)amino)methyl)-N′-methylbenzenesulfonimidamide

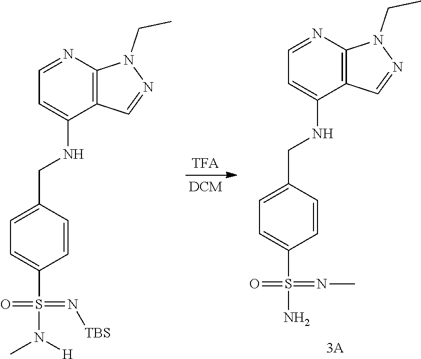

To a solution of N′-(tert-butyldimethylsilyl)-4-(((1-ethyl-1H-pyrazolo[3,4-b]pyridine-4-yl)amino)methyl)-N-methylbenzenesulfonimidamide (350 mg, 0.76 mmol) in DCM (10 mL) was added trifluoroacetic acid (3 mL) at room temperature. The reaction mixture was stirred at 25° C. for 1 hour. LCMS showed that the reaction worked completely, and that no starting material remained. The reaction mixture was then concentrated under reduced pressure to give a residue, which was re-dissolved in water (20 mL). The resulted solution was basified to pH=8 with a saturated solution of sodium bicarbonate in water and extracted with ethyl acetate (100 mL×3). The combined organic layers were washed with brine (50 mL), dried over anhydrous sodium sulfate, and filtered. The filtrate was concentrated under reduced pressure to give a crude product. The crude product was purified by Prep-HPLC (Column: Xbridge Prep OBD C18 Column, 30×150 mm, 5 m; Mobile Phase A: Water (10 mmol/L NH₄HCO₃), Mobile Phase: ACN; Flow rate: 60 mL/min; Gradient: 10% B to 30% B in 7 min, 30% B; Wave Length: 254 nm; RT: 6.10 min) to afford 4-(((1-ethyl-1H-pyrazolo[3,4-b]pyridine-4-yl)amino)methyl)-N′-methylbenzene sulfonimidamide (Compound 3A) (97.5 mg, 36%) as a white solid. LCMS (ESI) m/z=345.10 (M+H); ¹H NMR (300 MHz, DMSO-d6) δ 8.15 (s, 1H), 8.10-7.96 (m, 2H), 7.81 (d, J=8.3 Hz, 2H), 7.53 (d, J=8.1 Hz, 2H), 6.67 (brs, 1H), 6.10 (d, J=5.5 Hz, 1H), 4.61 (d, J=6.1 Hz, 2H), 4.34 (q, J=7.2 Hz, 2H), 4.19-4.03 (m, 1H), 2.34 (s, 3H), 1.36 (t, J=7.2 Hz, 3H).

Compounds 3B and 3C, listed below, were prepared according to similar procedures as described herein, particularly as described for Compound 3 (m/z means m/z (ES+)(M+H)⁺).

TABLE 3A

| Cmpd. | | | | |
|---|---|---|---|---|
| No. | Structure | Name | ¹H NMR | m/z |
| 3B | 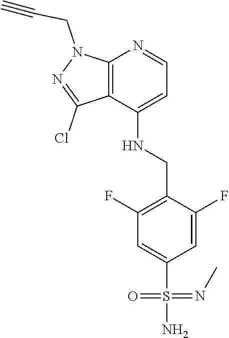 [SMILES: C#CCn1nc(Cl)c2c(NCc3c(F)cc(S(N)(=O)=NC)cc3F)ccnc21] | 4-(((3-chloro-1-(prop-2-yn- 1-y1)-1H-pyrazolo[3,4- b]pyridine-4- yl)amino)methyl)-3,5- difluoro-N- methylbenzene- sulfonimidamide | (300 MHz, DMSO-d₆) δ 8.15-8.14 (m, 1H), 7.51 (d, J -6.6 Hz, 2H), 7.05-6.94 (m, 2H), 6.34- 6.33 (m, 1H), 5.13 (s, 2H), 4.81-4.56 (m, 3H), 3.33 (s, 1H), 2.63-2.38 (m, 3H). | 424.95 |
| 3C | 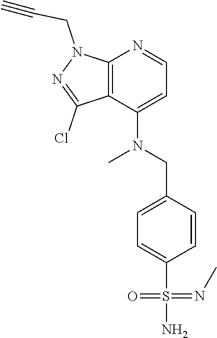 [SMILES: C#CCn1nc(Cl)c2c(N(C)Cc3ccc(S(N)(=O)=NC)cc3)ccnc21] | 4-(((3-chloro-1-(prop-2-yn- 1-y1)-1H-pyrazolo[3,4- b]pyridine-4- y1)(methyl)amino) methyl)-N′- methylbenzene- sulfonimidamide | (300 MHz, DMSO-d₆) δ 8.25-8.23 (m, 1H), 7.83- 7.80 (m, 2H), 7.44-7.41 (m, 2H), 6.58-6.56 (m, 1H), 5.12 (s, 2H), 4.87- 4.78 (m, 2H), 3.70-3.10 (m, 1H), 3.07 (s, 3H), 2.34 (s, 3H). | 403.05 |

### Synthesis of N′-cyano-4-(((1-ethyl-1H-pyrazolo[3,4-b]pyridine-4-yl)amino)methyl)benzenesulfonimidamide (Compound 3D)

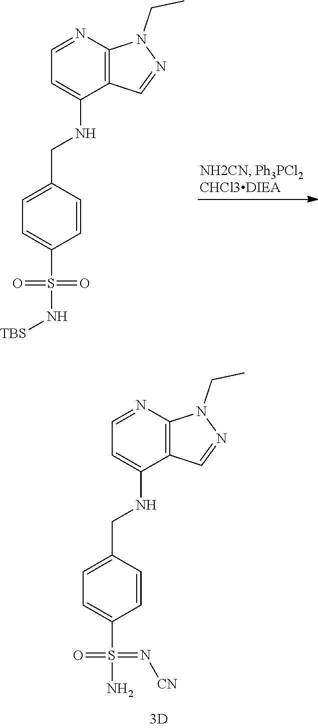

To a stirred mixture of triphenyldichlorophosphorane (1.12 g, 3.36 mmol) in chloroform (30 mL) under nitrogen atmosphere was added N,N-diisopropylethylamine (2.28 g, 11.19 mmol) at 0° C. After stirring at room temperature for 20 minutes, N-(tert-butyldimethylsilyl)-4-(((1-ethyl-1H-pyrazolo[3,4-b]pyridine-4-yl)amino)methyl) (see Ex. 3A, 500 mg, 1.12 mmol) was added at 0° C. The reaction mixture was stirred for 30 minutes, followed by an addition of cyanamide (252 mg, 6.72 mmol) at 0° C. The resulted mixture was stirred for 30 minutes at 0° C. and then 30 minutes at room temperature. The reaction mixture was quenched with water (50 mL), extracted with dichloromethane (50 mL×3), and the combined organic layers washed with water (50 mL) and brine (50 mL), dried over anhydrous sodium sulfate, filtered, and concentrated under vacuum. The residue was purified by column chromatography on silica gel eluting with methanol in dichloromethane (15%/85%). The product was further purified by Prep-HPLC (column: Xbridge Prep OBD C18 Column, 30×150 mm, 5 m; Mobile Phase A: Water (10 mmol/L NH₄HCO₃+0.1% NH₃·H₂O), Mobile Ise B: ACN; Flow rate: 60 mL/min; Gradient: 5% B to 30% B in 7 min; Wave Length: 254 nm; RT: 6.0 mi′) to give N′-cyano-4-(((1-ethyl-1H-pyrazolo[3,4-b]pyridine-4-yl)amino)methyl)benzenesulfonimidamide (Compound 3D) (72.4 mg, 18%) as a white solid. LCMS (ESI) m/z=356.05 (M+H); ¹H NMR (400 MHz, DMSO-d6) δ 8.15 (s, 1H), 8.10-7.91 (m, 2H), 7.79 (d, J=8.4 Hz, 2H), 7.47 (d, J=8.4 Hz, 2H), 6.33 (brs, 2H), 6.09 (d, J=5.2 Hz, 1H), 4.58 (d, J=6.0 Hz, 2H), 4.34 (q, J=7.2 Hz, 2H), 1.36 (t, J=7.2 Hz, 3H).

Compounds 3E and 3F, listed below, were prepared according to similar procedures as described herein (m/z means m/z (ES+)(M+H)⁺).

TABLE 3B

| Cmpd. | | | | |
|---|---|---|---|---|
| No. | Structure | Name | ¹H NMR | m/z |
| 3E | 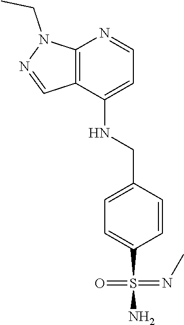 [SMILES: CCn1ncc2c(NCc3ccc([S@@](N)(=O)=NC)cc3)ccnc21] | (S)-4-(((1-ethy1-1H-pyrazolo[3,4- b]pyridine-4-yl)amino)methyl)-N′- methylbenzenesulfonimidamide | (300 MHz, DMSO-d₆) δ 8.14 (s, 1H), 8.01-7.95 (m, 2H), 7.85-7.79 (m, 2H), 7.53-7.51 (m, 2H), 6.65 (s, 1H), 6.10-6.05 (m, 1H), 4.61- 4.59 (m, 2H), 4.36-4.30 (m, 2H), 4.09 (s, 1H), 2.50-2.33 (m, 3H), 1.37-1.33 (m, 3H). | 345.15 |
| 3F | 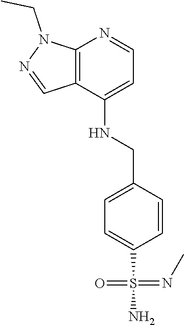 [SMILES: CCn1ncc2c(NCc3ccc([S@](N)(=O)=NC)cc3)ccnc21] | (R)-4-(((1-ethyl-1H-pyrazolo[3,4- b]pyridine-4-y1)amino)methyl)-N′- methylbenzenesulfonimidamide | (300 MHz, DMSO-d₆) δ 8.14 (s, 1H), 8.01-7.96 (m, 2H), 7.85-7.79 (m, 2H), 7.53-7.51 (m, 2H), 6.65 (s, 1H), 6.10-6.08 (m, 1H), 4.61- 4.59 (m, 2H), 4.36-4.31 (m, 2H), 4.09 (s, 1H), 2.50-2.33 (m, 3H), 1.37-1.33 (m, 3H). | 345.15 |

### Example 4

### Synthesis of 1-ethyl-N-((1-(S-methylsulfonimidoyl)piperidin-4-yl)methyl)-1H-pyrazolo[3,4-b]pyridine-4-amine (Compound 4A)

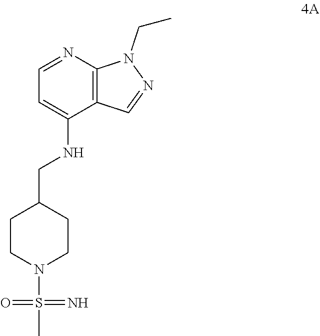 [SMILES: CCn1ncc2c(NCC3CCN(S(C)(=N)=O)CC3)ccnc21]

### Step 1: N-(tert-butyldimethylsilyl)methanesulfonamide

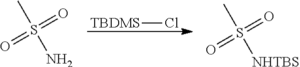

To a stirred mixture of methanesulfonamide (5.0 g, 52.56 mmol) and TBDMSCl (8.7 g, 57.82 mmol) in CHCl₃ (100 mL) at 0° C. was added TEA (8.0 g, 78.85 mmol). The reaction was stirred at 20° C. for 2 days. The reaction mixture was then quenched with water (200 mL) and extracted with ethyl acetate (100 mL×5). The combined organic layers were washed with brine (300 mL), dried over Na₂SO₄, and concentrated under vacuum to give N-(tert-butyldimethylsilyl) methanesulfonamide (9.3 g, 84%) as a light-yellow solid. ¹H NMR (400 MHz, Chloroform-d) δ 4.44 (s, 1H), 3.01 (s, 3H), 0.94 (s, 9H), 0.29 (s, 6H).

### Step 2: N-((1-(N-(tert-butyldimethylsilyl)-S-methylsulfonimidoyl)piperidin-4-yl)methyl)-1-ethyl-1H-pyrazolo[3,4-b]pyridine-4-amine

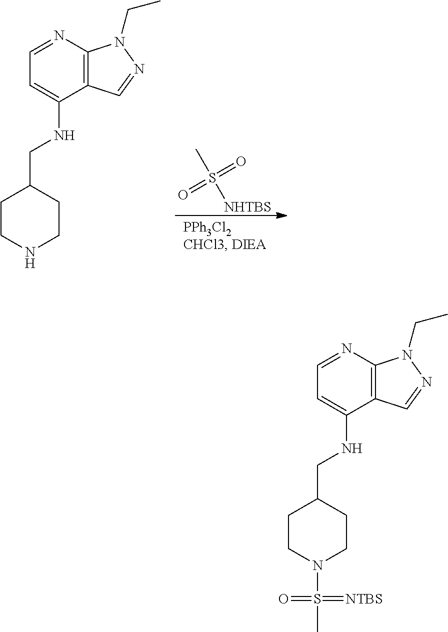

PPh₃Cl₂ (943 mg, 2.83 mmol) was dissolved in CHCl₃ (10 mL), then 106yri (1.22 g, 9.43 mmol) was added at 0° C. The reaction mixture was stirred under N₂ at 0° C. for 1 h. A solution of N-(tert-butyldimethylsilyl)methanesulfonamide (395 mg, 1.89 mmol) in CHCl₃ (2 mL) was added dropwise. After stirring at 0° C. for 1 h, a solution of 1-ethyl-N-(piperidin-4-ylmethyl)-1H-pyrazolo[3,4-b]pyridine-4-amine (600 mg, 2.03 mmol) in CHCl₃ (15 mL) was added dropwise. The reaction mixture was stirred at 20° C. for 2 h. The reaction mixture was quenched with water (50 mL) and extracted with ethyl acetate (60 mL×3). The combined organic layers were washed with brine (100 mL), dried over Na₂SO₄, and concentrated under vacuum. The resulted residue was purified by column chromatography (silica gel, ethyl acetate:petroleum ether=0%: 100% to 30%: 70%) to afford N-((1-(N-(tert-butyldimethylsilyl)-S-methylsulfonimidoyl)piperidin-4-yl)methyl)-1-ethyl-1H-pyrazolo[3,4-b]pyridine-4-amine (510 mg, 55%) as a white solid. LCMS (ESI) m/z=451.15 (M+H).

### Step 3: 1-ethyl-N-((1-(S-methylsulfonimidoyl)piperidin-4-yl)methyl)-1H-pyrazolo[3,4-b]pyridine-4-amine

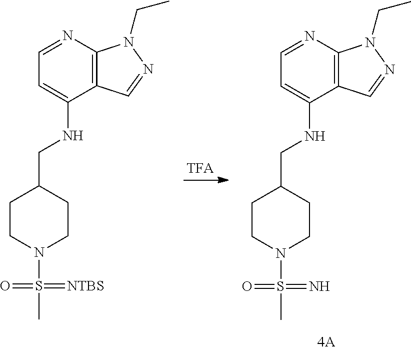

A mixture of N-((1-(N-(tert-butyldimethylsilyl)-S-methylsulfonimidoyl)piperidin-4-yl)methyl)-1-ethyl-1H-pyrazolo[3,4-b]pyridine-4-amine (440 mg, 0.98 mmol) in TFA (8 mL) was stirred at 20° C. for 1 h. The volatiles were then removed under vacuum and the resulted residue was purified by Prep-HPLC (Column: Xbridge Prep OBD C18 Column, 30×150 mm, 5 μm; Mobile Phase A: Water (10 mmol/L NH₄HCO₃+0.1% NH₃·H₂O), Mobile Phase B: ACN; Flow rate: 60 mL/min; Gradient: 8% B to 38% B in 7 min; Wave Length: 254 nm; RT (min): 5.12) to afford 1-ethyl-N-((1-(S-methylsulfonimidoyl)piperidin-4-yl)methyl)-1H-pyrazolo[3,4-b]pyridine-4-amine (Compound 4A) (97 mg, 29%) as a white solid. LCMS (ESI) m/z=337.10 (M+H); ¹H NMR (400 MHz, DMSO-d₆) δ 8.12 (s, 1H), 7.99 (d, J=5.2 Hz, 1H), 7.38 (t, J=5.6 Hz, 1H), 6.17 (d, J=5.6 Hz, 1H), 4.32 (q, J=7.2 Hz, 2H), 3.87-3.60 (m, 2H), 3.46 (s, 1H), 3.19 (t, J=6.0 Hz, 2H), 2.70 (s, 3H), 2.62-2.52 (m, 2H), 1.85 (d, J=12.4 Hz, 2H), 1.77-1.64 (m, 1H), 1.34 (t, J=7.2 Hz, 3H), 1.29-1.13 (m, 2H).

### Example 5

### Synthesis of N′-(cyclopropylmethyl)-4-((1-ethyl-1H-pyrazolo[3,4-b]pyridine-4-yl)amino)benzenesulfonimidamide (Compound 5A)

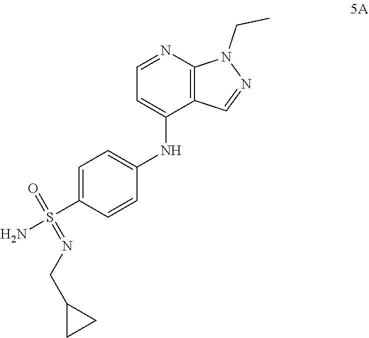 [SMILES: CCn1ncc2c(Nc3ccc(S(N)(=O)=NCC4CC4)cc3)ccnc21]

### Step 1: 4-chloro-1-ethyl-1H-pyrazolo[3,4-b]pyridine

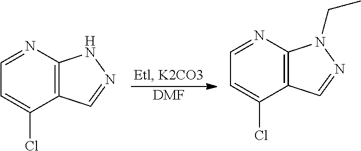

To a mixture of 4-chloro-1H-pyrazolo[3,4-b]pyridine (5.0 g, 32.56 mmol) and potassium carbonate (13.50 g, 97.68 mmol) in DMF (100 mL) was added iodoethane (10.16 g, 65.12 mmol) at 0° C. After stirring at room temperature for 2 hours, the reaction mixture was quenched with water (300 mL) and extracted with ethyl acetate (300 mL×3). The combined organic layers were washed with water (200 mL×2) and brine (200 mL×3) and dried over Na₂SO₄. The solid was filtered off and the filtrate was concentrated under reduced pressure. The residue was purified by column chromatography (silica gel, ethyl acetate:petroleum ether=0%: 100% to 10%: 90%) to give 4-chloro-1-ethyl-pyrazolo[3,4-b]pyridine (4.4 g, 72%) as yellow oil. LCMS (ESI) m/z=182.15, 184.15 (M+H).

### Step 2: 4-((1-ethyl-1H-pyrazolo[3,4-b]pyridine-4-yl)amino)benzenesulfonamide

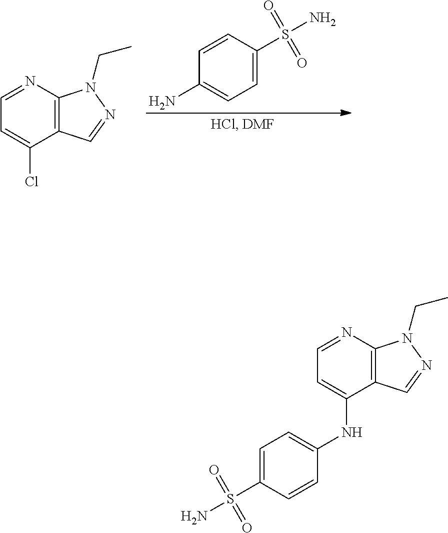 [SMILES: CCn1ncc2c(Cl)ccnc21 ; Nc1ccc(S(N)(=O)=O)cc1 ; CCn1ncc2c(Nc3ccc(S(N)(=O)=O)cc3)ccnc21]

To a mixture of 4-chloro-1-ethyl-pyrazolo[3,4-b]pyridine (500 mg, 2.75 mmol) and 4-aminobenzenesulfonamide (1.42 g, 8.26 mmol) in DMF (10 mL) was added conc. HCl (cat.). The reaction mixture was stirred at 130° C. for 15 hours. After cooling to room temperature, the reaction mixture was quenched with saturated aqueous sodium carbonate (10 mL) and extracted with ethyl acetate (50 mL×3). The combined organic layers were washed with water (50 mL×2) and brine (50 mL×3), then dried over Na₂SO₄. The solid was filtered off and the filtrate was concentrated under reduced pressure to give 4-((1-ethyl-1H-pyrazolo[3,4-b]pyridine-4-yl)amino)benzenesulfonamide (900 mg, crude) as a white solid. The crude product was used directly in next step without purification. LCMS (ESI) m/z=318.00 (M+H).

### Step 3: N-(tert-butyldimethylsilyl)-4-((1-ethyl-1H-pyrazolo[3,4-b]pyridine-4-yl)amino)benzenesulfonamide

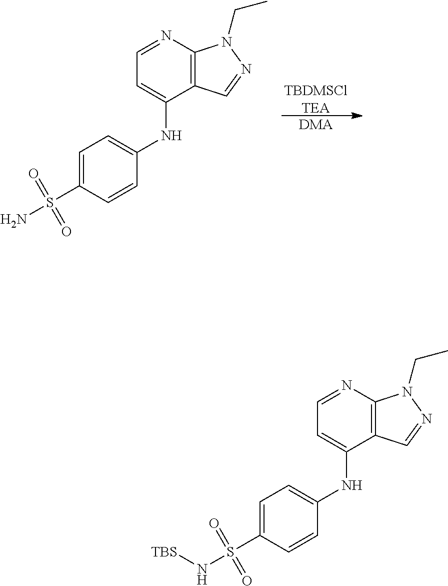

To a solution of 4-((1-ethyl-1H-pyrazolo[3,4-b]pyridine-4-yl)amino)benzenesulfonamide (900 mg, crude) in N,N-dimethylacetamide (20 mL) at 0° C. were added triethylamine (286 mg, 2.84 mmol) and tert-butyldimethylsilyl chloride (427 mg, 2.84 mmol). After stirring at room temperature for 15 hours, the reaction mixture was quenched with water (50 mL) and extracted with ethyl acetate (100 mL×3). The combined organic layers were washed with water (50 mL×2) and brine (50 mL×2), dried over Na₂SO₄. The solid was filtered off and the filtrate was concentrated under reduced pressure to give N-(tert-butyldimethylsilyl)-4-((1-ethyl-1H-pyrazolo[3,4-b]pyridine-4-yl)amino)benzenesulfonamide (650 mg, crude) as yellow oil. The crude product was used directly in next step without purification. LCMS (ESI) m/z=432.10 (M+H).

### Step 4: N′-(tert-butyldimethylsilyl)-N-(cyclopropylmethyl)-4-((1-ethyl-1H-pyrazolo [3,4-d]pyrimidin-4-yl)amino)benzenesulfonimidamide

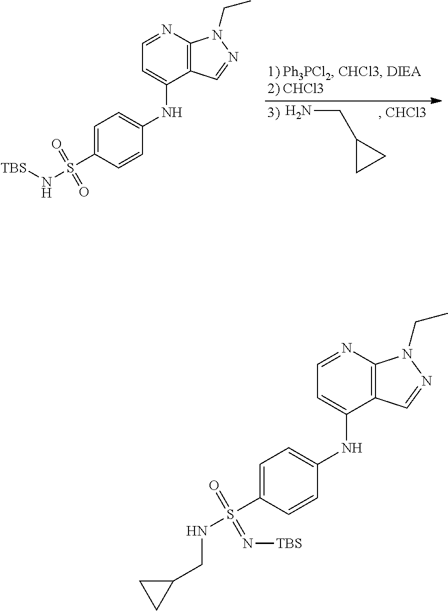

To a solution of triphenylphosphine dichloride (1.51 g, 4.52 mmol) in chloroform (6 mL) at 0° C. and under a N₂ atmosphere, was added N,N-diisopropylethylamine (2.14 g, 10.54 mmol). The reaction mixture was stirred at room temperature for 10 minutes. A solution of N-(tert-butyldimethylsilyl)-4-((1-ethyl-1H-pyrazolo[3,4-b]pyridine-4-yl)amino)benzenesulfonamide (650 mg, crude) in chloroform (2 mL) was added at 0° C. and the reaction mixture was stirred at 0° C. for 20 minutes. A solution of cyclopropylmethanamine (642 mg, 9.04 mmol) in chloroform (2 mL) was added to the reaction mixture at 0° C. The resulting mixture was stirred at 0° C. for 30 minutes and then room temperature for 30 minutes. The reaction mixture was quenched with water (150 mL) and extracted with DCM (200 mL×3). The combined organic layers were washed (100 mL×2) with water and brine (100 mL×2) and dried over Na₂SO₄. The solid was filtered off and the filtrate was concentrated under reduced pressure. The residue was purified (silica gel; eluting with 0 to 100% ethyl acetate in petroleum ether) to give a crude product, which was further purified by reverse-phase column chromatography (eluting with 10-100% acetonitrile in water) to afford N′-(tert-butyldimethylsilyl)-N-(cyclopropylmethyl)-4-((1-ethyl-1H-pyrazolo[3,4-d]pyrimidin-4-yl)amino)benzene sulfonimidamide (250 mg, 33%) as a white solid. LCMS (ESI) m/z=486.15 (M+H).

### Step 5: N′-(cyclopropylmethyl)-4-((1-ethyl-1H-pyrazolo[3,4-b]pyridine-4-yl)amino)benzenesulfonimidamide

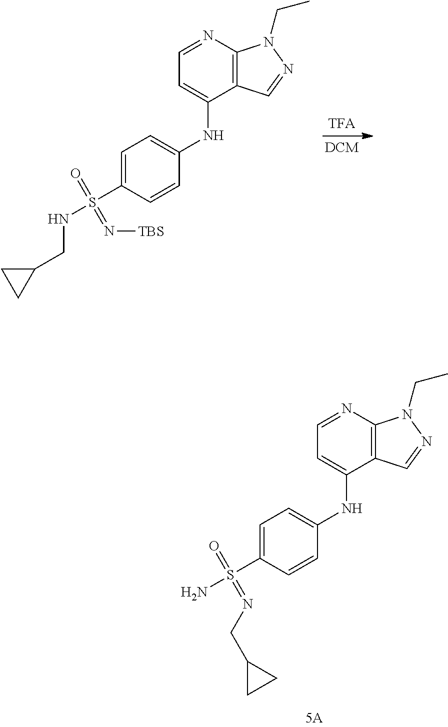

To a solution of N′-(tert-butyldimethylsilyl)-N-(cyclopropylmethyl)-4-((1-ethyl-1H-pyrazolo[3,4-d]pyrimidin-4-yl)amino)benzenesulfonimidamide (250 mg, 0.52 mmol) in DCM (6 mL) was added trifluoroacetic acid (2 mL). After stirring at room temperature for 1 hour, the reaction mixture was concentrated under reduced pressure. The residue was purified by reverse-phase Prep-HPLC (Column: Xbridge Shield RP18 OBD Column, 30×150 mm, 5 m; Mobile Phase A: Water (10 mmol/L NH₄HCO₃), Mobile Phase B: ACN; Flow rate: 60 mL/min; Gradient: 22% B to 42% B in 7 min; Wave Length: 254 nm; RT (mi′): 5.2) to give N′-(cyclopropylmethyl)-4-((1-ethyl-1H-pyrazolo[3,4-b]pyridine-4-yl)amino)benzenesulfonimidamide (Compound 5A) (88.6 mg, 45%) as a white solid. LCMS (ESI) m/z=371.05 (M+H); ¹H NMR (300 MHz, DMSO-d₆) δ 9.57 (s, 1H), 8.21-8.17 (m, 2H), 7.86 (d, J=8.7 Hz, 2H), 7.44 (d, J=8.7 Hz, 2H), 6.94 (s, 1H), 6.83 (d, J=5.4 Hz, 1H), 4.42 (q, J=7.2 Hz, 2H), 4.04 (s, 1H), 2.63 (d, J=6.8 Hz, 2H), 1.40 (t, J=7.2 Hz, 3H), 0.86-0.73 (m, 1H), 0.36-0.25 (m, 2H), 0.10-0.05 (m, 2H).

### Synthesis of Compounds 5B-5I

Compounds 5B-5I, listed below, were prepared according to similar procedures as described herein (m/z means m/z (ES+)(M+H)⁺).

TABLE 5

| Cmpd. | | | | |
|---|---|---|---|---|
| No. | Structure | Name | ¹H NMR | m/z |
| 5B | 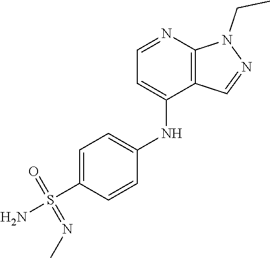 [SMILES: CCn1ncc2c(Nc3ccc(S(N)(=O)=NC)cc3)ccnc21] | 4-((1-ethy1-1H- pyrazolo [3,4-b]pyridine-4- y1)amino)-N′-methylbenzene sulfonimidamide | (300 MHz, DMSO-d6) δ ppm 9.90 (s, 1 H), 8.27 (d, J = 5.50 Hz, 1H), 8.21 (s, 1H), 7.92 (d, J = 8.80 Hz, 2H), 7.56 (d, J = 8.80 Hz, 2H), 6.93 (d, J = 5.50 Hz, 1H), 4.44 (q, J = 7.21 Hz, 2H), 2.53 (s, 3H), 1.41 (t, J = 7.15 Hz, 3H). | 331.05 |
| 5C | 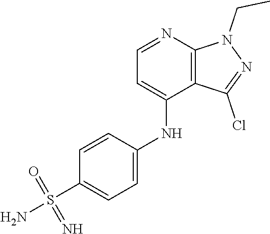 [SMILES: CCn1nc(Cl)c2c(Nc3ccc(S(=N)(N)=O)cc3)ccnc21] | 4-((3-chloro-1-ethy1-1H- pyrazolo[3,4-b]pyridine-4- yl)amino)benzene sulfonimidamide | (300 MHz, DMSO-d6) δ: 9.02 (br s, 1H), 8.29-8.38 (m, 1H), 7.89-7.97 (m, 3H), 7.47-7.57 (m, 2H), 6.84-6.95 (m, 1H), 4.33-4.49 (m, 2H), 1.33-1.46 (m, 3H). | 351.00 |
| 5D | 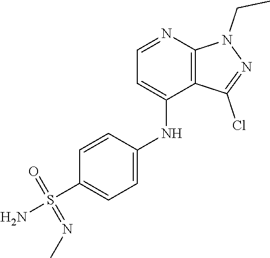 [SMILES: CCn1nc(Cl)c2c(Nc3ccc(S(N)(=O)=NC)cc3)ccnc21] | 4-((3-chloro-1-ethy1-1H- pyrazolo[3,4-b]pyridine-4- yl)amino)-N′-methylbenzene sulfonimidamide | (300 MHz, DMSO-d6) δ: 8.26 (d, J = 5.5 Hz, 1H), 7.83 (d, J = 8.6 Hz, 2H), 7.49 (d, J = 8.6 Hz, 2H), 6.83 (d, J = 5.5 Hz, 2H), 4.40 (q, J = 7.2 Hz, 2H), 2.38 (s, 3H), 1.40 (t, J = 7.2 Hz, 3H). | 365.00 |
| 5E | 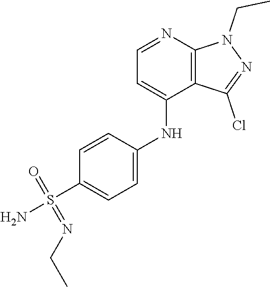 [SMILES: CCN=S(N)(=O)c1ccc(Nc2ccnc3c2c(Cl)nn3CC)cc1] | 4-((3-chloro-1-ethyl-1H- pyrazolo[3,4-b]pyridine-4- yl)amino)-N′-ethylbenzene sulfonimidamide | (300 MHz, DMSO-d6) δ: 8.71 (br s, 1H), 8.25 (d, J = 5.3 Hz, 1H), 7.84 (d, J = 8.8 Hz, 2H), 7.48 (d, J = 8.6 Hz, 2H), 6.82 (d, J = 5.3 Hz, 1H), 6.72 (br s, 1H), 4.40 (q, J = 7.2 Hz, 2H), 4.07 (br s, 1H), 2.76 (q, J = 7.1 Hz, 2H), 1.40 (t, J = 7.2 Hz, 3H), 0.96 (t, J = 7.2 Hz, 3H). | 379.00 |
| 5F | 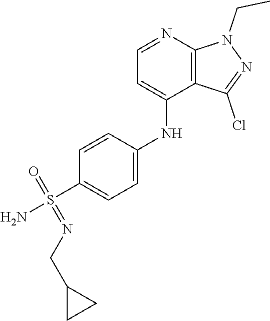 [SMILES: CCn1nc(Cl)c2c(Nc3ccc(S(N)(=O)=NCC4CC4)cc3)ccnc21] | 4-((3-chloro-1-ethyl-1H- pyrazolo[3,4-b]pyridine- 4-yl)amino)-N′- (cyclopropylmethyl) benzene sulfonimidamide | (300 MHz, DMSO-d6₎ δ ppm 8.25 (d, J = 5.50 Hz, 1H), 7.85 (d, J = 8.62 Hz, 2H), 7.46 (d, J = 8.62 Hz, 2H), 6.80 (d, J = 5.32 Hz, 1H), 4.40 (q, J = 7.21 Hz, 2H), 2.62 (d, J = 6.79 Hz, 2H), 1-40 (s, 3H), 0.71- 0-90 (m, 1H), 0.28-0.40 (m, 2H), 0.08 (q, J = 4.83 Hz, 2H). | 405.05 |
| 5G | 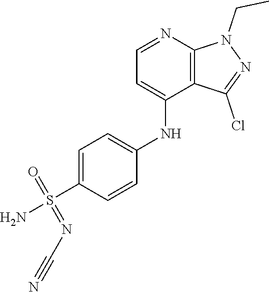 [SMILES: CCn1nc(Cl)c2c(Nc3ccc(S(N)(=O)=NC#N)cc3)ccnc21] | 4-((3-chloro-1-ethy1-1H- pyrazolo[3,4-b]pyridine-4- yl)amino)-N′-cyano benzenesulfonimidamide | (300 MHz, DMSO-d6) δ ppm 9.06 (s, 1 H) 8.34 (d, J = 5.50 Hz, 1 H) 7.88 (d, J = 8.80 Hz, 2 H) 7.56 (d, J = 8.99 Hz, 2 H) 6.97 (d, J = 5.32 Hz, 1 H) 4.42 (q, J = 7.15 Hz, 2 H) 1.41 (t, J = 7.24 Hz, 3 H) | 376 |
| 5H | 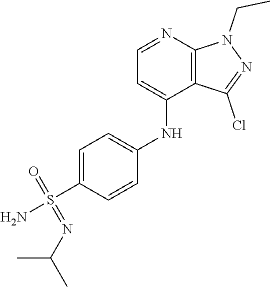 [SMILES: CCn1nc(Cl)c2c(Nc3ccc(S(N)(=O)=NC(C)C)cc3)ccnc21] | 4-((3-chloro-1-ethyl-1H- pyrazolo[3,4-b]pyridine-4- yl)amino)-N′- isopropylbenzene- sulfonimidamide | (300 MHz, DMSO-d6) δ 9.89-9.71 (m, 2H), 8.42-8.40 (m, 1H), 8.14-8.01 (m, 2H), 7.58-7.55 (m, 2H), 7.03-7.01 (m, 1H), 4.48-4.41 (m, 2H), 3.63-3.59 (m, 1H), 1.50-1.39 (m, 3H), 1.08-1.06 (m, 3H), 0.98-0.95 (m, 3H). | 393.05 |
| 5I | 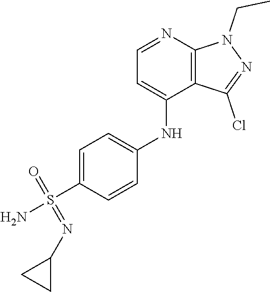 [SMILES: CCn1nc(Cl)c2c(Nc3ccc(S(N)(=O)=NC4CC4)cc3)ccnc21] | 4-((3-chloro-1-ethy1-1H- pyrazolo[3,4-b]pyridine-4- yl)amino)-N′- cyclopropylbenzene- sulfonimidamide | (300 MHz, DMSO-d6) δ 8.75-8.67 (m, 1H), 8.26-8.24 (m, 1H), 7.88-7.72 (m, 2H), 7.49-7.47 (m, 2H), 7.39-6.95 (m, 1H), 6.83-6.81 (m, 1H), 4.50-4.38 (m, 2H), 4.36- 4.3.97 (m, 0.5H), 2.21-2.14 (m, 1H), 1.42-1.21 (m, 3H), 0.42-0.36 (m, 4H). | 391.05 |

### Example 6

### Synthesis of (R)-(4-(((3-chloro-1-(prop-2-yn-1-yl)-1H-pyrazolo[3,4-b]pyridine-4-yl)amino)methyl)phenyl)(imino)(methyl)-λ⁶-sulfanone (Compound 6A)

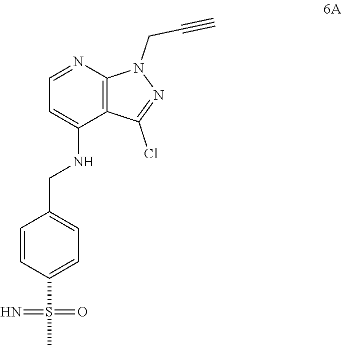

### And

### (S)-(4-(((3-chloro-1-(prop-2-yn-1-yl)-1H-pyrazolo[3,4-b]pyridine-4-yl)amino)methyl)phenyl)(imino)(methyl)-λ⁶-sulfanone (Compound 6B)

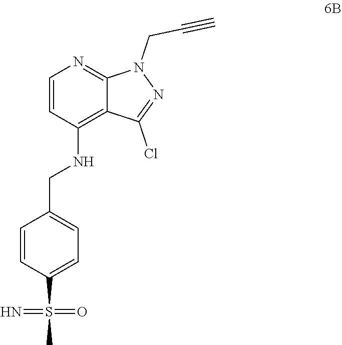 [SMILES: C#CCn1nc(Cl)c2c(NCc3ccc([S@@](C)(=N)=O)cc3)ccnc21]

### Step 1: 3-chloro-N-(4-(methylthio)benzyl)-1-(3-(trimethylsilyl)prop-2-yn-1-yl)-1H-pyrazolo[3,4-b]pyridine-4-amine

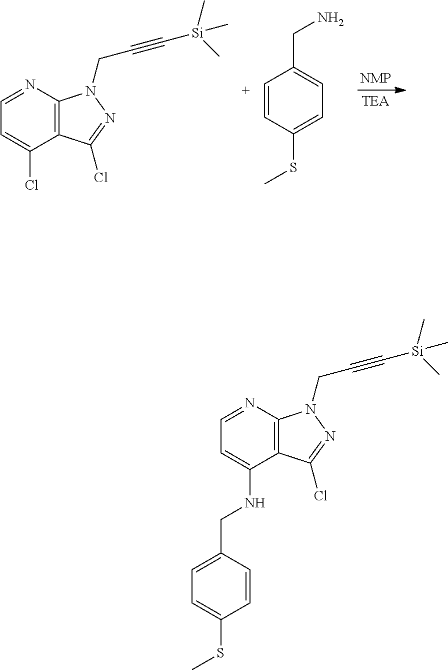

To a solution of 3,4-dichloro-1-(3-(trimethylsilyl)prop-2-yn-1-yl)-1H-pyrazolo[3,4-b]pyridine (1.5 g, 5.03 mmol) and (4-(methylthio)phenyl)methanamine (1.54 g, 10.06 mmol) in NMP (20 mL) was added triethylamine (1.53 g, 15.09 mmol). The reaction mixture was stirred at 130° C. for 3 h. The cooled reaction mixture was quenched with water (100 mL) and extracted with ethyl acetate (100 mL×3). The organic layers were combined, washed with brine (120 mL), dried over sodium sulfate, filtered, and concentrated under vacuum. The residue was purified (silica gel, ethyl acetate:petroleum ether=56%: 44%) to give 3-chloro-N-(4-(methylthio)benzyl)-1-(3-(trimethylsilyl)prop-2-yn-1-yl)-1H-pyrazolo[3,4-b]pyridine-4-amine (1.65 g, 77%) as a yellow oil. LCMS (ESI) m/z=415.05, 417.05 (M+H); ¹H NMR (400 MHz, DMSO-d₆) δ 8.02 (d, J=5.6 Hz, 1H), 7.37-7.29 (m, 3H), 7.23 (d, J=8.3 Hz, 2H), 6.15 (d, J=5.7 Hz, 1H), 5.17 (s, 2H), 4.53 (d, J=6.1 Hz, 2H), 2.44 (s, 3H), 0.12 (s, 9H).

### Step 2: 3-chloro-N-(4-(methylsulfinyl)benzyl)-1-(3-(trimethylsilyl)prop-2-yn-1-yl)-1H-pyrazolo[3,4-b]pyridine-4-amine

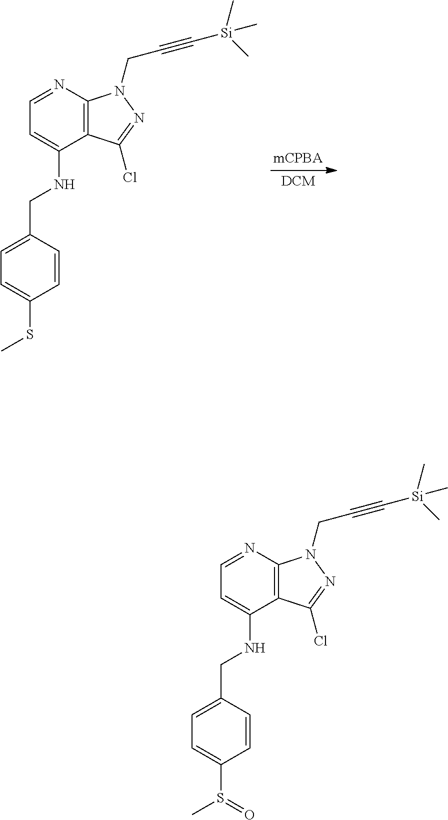 [SMILES: CSc1ccc(CNc2ccnc3c2c(Cl)nn3CC#C[Si](C)(C)C)cc1 ; CS(=O)c1ccc(CNc2ccnc3c2c(Cl)nn3CC#C[Si](C)(C)C)cc1]

To a solution of 3-chloro-N-(4-(methylthio)benzyl)-1-(3-(trimethylsilyl)prop-2-yn-1-yl)-1H-pyrazolo[3,4-b]pyridine-4-amine (1.6 g, 3.86 mmol) in DCM (20 mL) was added 3-chlorobenzoperoxoic acid (783 mg, 3.86 mmol, 85% purity). The reaction mixture was stirred at 20° C. for 5 h. The reaction mixture was diluted with DCM (60 mL) and then washed with saturated aqueous sodium thiosulfate (80 mL), saturated aqueous sodium bicarbonate (80 mL) and brine (80 mL). The organic layer was dried over sodium sulfate, filtered, and concentrated under vacuum. The residue was purified (silica gel; eluting with 7% methanol in dichloromethane) to give 3-chloro-N-(4-(methylsulfinyl)benzyl)-1-(3-(trimethylsilyl)prop-2-yn-1-yl)-1H-pyrazolo[3,4-b]pyridine-4-amine (1.35 g, 80%) as a white solid. LCMS (ESI) m/z=431.00, 433.00 (M+H); ¹H NMR (400 MHz, DMSO-d₆) δ 8.03 (d, J=5.6 Hz, 1H), 7.66 (d, J=8.4 Hz, 2H), 7.58 (d, J=8.3 Hz, 2H), 7.43 (t, J=6.3 Hz, 1H), 6.17 (d, J=5.7 Hz, 1H), 5.18 (s, 2H), 4.66 (d, J=6.3 Hz, 2H), 2.72 (s, 3H), 0.12 (s, 9H).

### Step 3: (4-(((3-chloro-1-(3-(trimethylsilyl)prop-2-yn-1-yl)-1H-pyrazolo[3,4-b]pyridine-4-yl)amino)methyl)phenyl)(imino)(methyl)-λ⁶-sulfanone

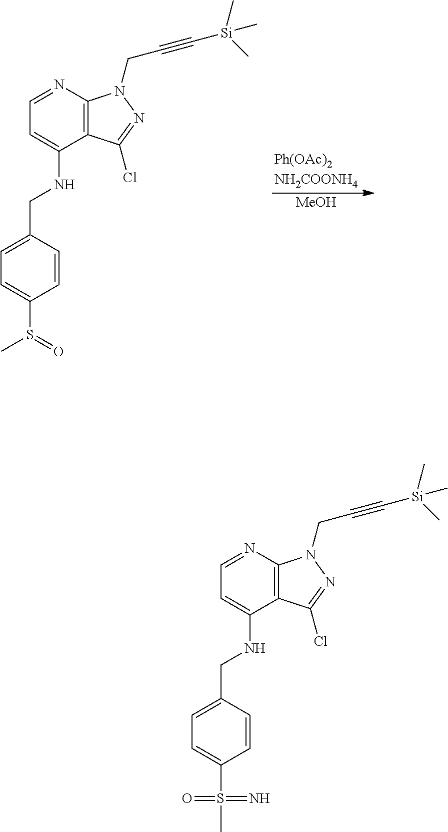 [SMILES: "MeOH" ; CS(=O)c1ccc(CNc2ccnc3c2c(Cl)nn3CC#C[Si](C)(C)C)cc1 ; C[Si](C)(C)C#CCn1nc(Cl)c2c(NCc3ccc(S(C)(=N)=O)cc3)ccnc21]

To a solution of 3-chloro-N-(4-(methylsulfinyl)benzyl)-1-(3-(trimethylsilyl)prop-2-yn-1-yl)-1H-pyrazolo[3,4-b]pyridine-4-amine (1.25 g, 2.90 mmol) in methanol (20 mL) were added PhI(Oac)₂ (2.80 g, 8.70 mmol) and ammonium carbamate (906 mg, 11.60 mmol). The reaction mixture was stirred at 20° C. for 1 h. The reaction mixture was quenched with saturated aqueous sodium bicarbonate (80 mL) and extracted with ethyl acetate (80 mL×3). The organic layers were combined, washed with brine (120 mL), dried over sodium sulfate, filtered, and concentrated under vacuum. The residue was purified (silica gel; eluting with 5% methanol in dichloromethane) to afford (4-(((3-chloro-1-(3-(trimethylsilyl)prop-2-yn-1-yl)-1H-pyrazolo[3,4-b]pyridine-4-yl)amino)methyl)phenyl)(imino)(methyl)-λ⁶-sulfanone (1.1 g, 71%) as a yellow solid. LCMS (ESI) m/z=446.05, 448.05 (M+H); ¹H NMR (300 MHz, DMSO-d₆) δ 8.03 (d, J=5.6 Hz, 1H), 7.89 (d, J=8.0 Hz, 2H), 7.59 (d, J=8.0 Hz, 2H), 7.46 (t, J=6.6 Hz, 1H), 6.13 (d, J 25=5.7 Hz, 1H), 5.18 (s, 2H), 4.68 (d, J=6.2 Hz, 2H), 3.05 (s, 3H), 0.13 (s, 9H). Step 4: (4-(((3-chloro-1-(prop-2-yn-1-yl)-1H-pyrazolo[3,4-b]pyridine-4-yl)amino)methyl)phenyl)(imino)(methyl)-λ⁶-sulfanone

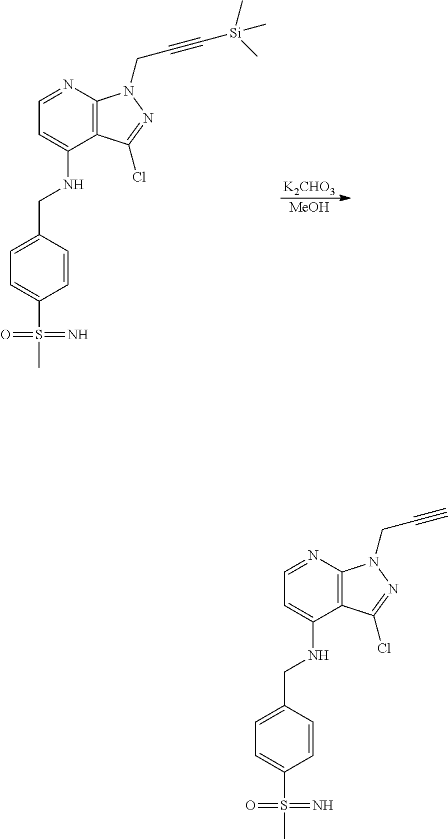 [SMILES: "MeOH" ; C[Si](C)(C)C#CCn1nc(Cl)c2c(NCc3ccc(S(C)(=N)=O)cc3)ccnc21 ; "K2CHO3" ; C#CCn1nc(Cl)c2c(NCc3ccc(S(C)(=N)=O)cc3)ccnc21]

To a mixture of (4-(((3-chloro-1-(3-(trimethylsilyl)prop-2-yn-1-yl)-1H-pyrazolo[3,4-b]pyridine-4-yl)amino)methyl)phenyl)(imino)(methyl)-λ⁶-sulfanone (400 mg, 0.90 mmol) in methanol (10 mL) was added potassium carbonate (372 mg, 2.69 mmol). The reaction mixture was stirred at 20° C. for 3 h. The reaction mixture was quenched with water (50 mL) and then extracted with ethyl acetate (50 mL×3). The organic layers were combined, washed with brine (50 mL), dried over sodium sulfate, filtered, and concentrated under vacuum. The residue was purified (Prep-TLC; eluting with 6% methanol in dichloromethane) to give (4-(((3-chloro-1-(prop-2-yn-1-yl)-1H-pyrazolo[3,4-b]pyridine-4-yl)amino)methyl)phenyl)(imino)(methyl)-λ⁶-sulfanone (300 mg, 0.79 mmol, 89%) as a white solid. LCMS (ESI) m/z=373.95, 375.95 (M+H); H NMR (300 MHz, DMSO-d₆) δ 8.03 (d, J=5.6 Hz, 1H), 7.89 (d, J=8.4 Hz, 2H), 7.59 (d, J=8.4 Hz 2H), 7.45 (t, J=6.3 Hz, 1H), 6.14 (d, J=5.7 Hz, 1H), 5.13 (d, J=2.5 Hz, 2H), 4.69 (d, J 15=6.3 Hz, 2H), 4.15 (s, 1H), 3.36 (t, J=2.5 Hz, 1H), 3.04 (s, 3H).

### Step 5: (R)-(4-(((3-chloro-1-(prop-2-yn-1-yl)-1H-pyrazolo[3,4-b]pyridine-4-yl)amino)methyl)phenyl)(imino)(methyl)-λ⁶-sulfanone (6A) and (S)-(4-(((3-chloro-1-(prop-2-yn-1-yl)-1H-pyrazolo[3,4-b]pyridine-4-yl)amino)methyl)phenyl)(imino)(methyl)-λ⁶-sulfanone (6B)

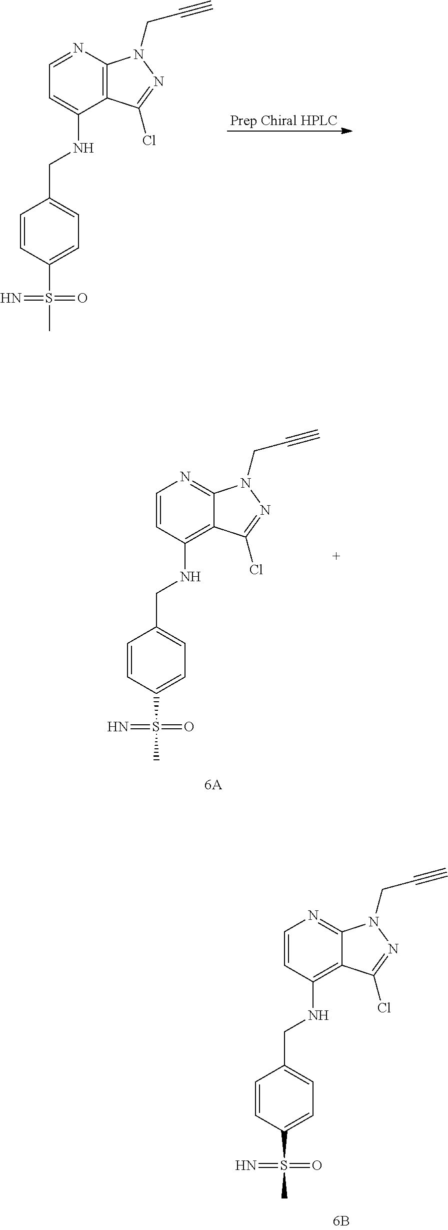 [SMILES: C#CCn1nc(Cl)c2c(NCc3ccc(S(C)(=N)=O)cc3)ccnc21 ; "Prep Chiral HPLC" ; C#CCn1nc(Cl)c2c(NCc3ccc([S@](C)(=N)=O)cc3)ccnc21 ; C#CCn1nc(Cl)c2c(NCc3ccc([S@@](C)(=N)=O)cc3)ccnc21]

(4-(((3-chloro-1-(prop-2-yn-1-yl)-1H-pyrazolo[3,4-b]pyridine-4-yl)amino)methyl) phenyl)(imino)(methyl)-λ⁶-sulfanone (100 mg, 0.27 mmol) was purified by Prep Chiral HPLC (Column: CHIRALPAK IC, 2×25 cm, 5 m; mobile Phase A: EtOH, Mobile Phase B: MTB (0.5% 2 M NH₃-MeOH); Flow rate: 20 mL/min; Gradient: 70% B to 70% B in 9 min; Wave Length: 220/254 nm).

### RT 5.94 min:

(R)-(4-(((3-chloro-1-(prop-2-yn-1-yl)-1H-pyrazolo[3,4-b]pyridine-4-yl)amino) methyl)phenyl)(imino)(methyl)-λ⁶-sulfanone (Compound 6A, 21.9 mg, 22%), white solid. LCMS (ESI) m/z=374.00, 376.00 (M+H); ¹H NMR (300 MHz, DMSO-d₆) δ 8.03 (d, J=5.4 Hz, 1H), 7.89 (d, J=8.1 Hz, 2H), 7.58 (d, J=8.1 Hz, 2H), 7.44 (t, J=6.3 Hz, 1H), 6.13 (d, J=5.7 Hz, 1H), 5.13 (s, 2H), 4.68 (d, J=6.3 Hz, 2H), 4.15 (s, 1H), 3.36 (s, 1H), 3.03 (s, 3H).

### RT 7.54 min:

(S)-(4-(((3-chloro-1-(prop-2-yn-1-yl)-1H-pyrazolo[3,4-b]pyridine-4-yl)amino) methyl)phenyl)(imino)(methyl)-λ⁶-sulfanone (Compound 6B, 16.1 mg, 16%), white solid. LCMS (ESI) m/z=373.95, 375.95 (M+H); ¹H NMR (300 MHz, DMSO-d₆) δ 8.03 (d, J=5.4 Hz, 1H), 7.88 (d, J=8.1 Hz, 2H), 7.58 (d, J=7.9 Hz, 2H), 7.44 (t, J=6.3 Hz, 1H), 6.13 (d, J=5.4 Hz, 1H), 5.13 (s, 2H), 4.68 (d, J=6.3 Hz, 2H), 4.15 (s, 1H), 3.35 (s, 1H), 3.03 (s, 3H).

### Example 7

### Synthesis of 4-(((1-ethyl-1H-pyrazolo[3,4-b]pyridine-4-yl)oxy)methyl)-N′-methylbenzenesulfonimidamide (Compound 7A)

4-(((1-ethyl-1H-pyrazolo[3,4-b]pyridine-4-yl)oxy)methyl)-N′-methylbenzene sulfonimidamide (Compound 7A), listed below, were prepared according to similar procedures as described herein (m/z means m/z (ES+)(M+H)⁺).

TABLE 7

| Cmpd. | | | | |
|---|---|---|---|---|
| No. | Structure | Name | ¹H NMR | m/z |
| 7A | 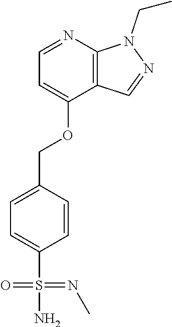 [SMILES: CCn1ncc2c(OCc3ccc(S(N)(=O)=NC)cc3)ccnc21] | 4-(((1-ethy1-1H- pyrazolo [3,4-b]pyridine- 4-yl)oxy)methyl)-N′- methylbenzene- sulfonimidamide | (300 MHz, DMSO-d6) δ 8.40 (d, J = 5.4 Hz, 1H), 8.20 (s, 1H), 7.90 (d, J = 8.4 Hz, 2H), 7.70 (d, J = 8.4 Hz, 2H), 6.83 (d, J = 7.6 Hz, 1H), 6.77 (s, br, 1H), 5.51 (s, 2H), 4.60 (q, J = 7.2 Hz, 2H), 4.24 (s, br, 1H), 2.38 (s, 3H), 1.41 (t, J = 7.2 Hz, 3H). | 346.10 |

### Example 8

### Synthesis of 2-(3-chloro-4-((4-(S-methylsulfonimidoyl)benzyl)oxy)-1H-pyrazolo[3,4-b]pyridine-1-yl)acetonitrile (Compound 8A)

2-(3-chloro-4-((4-(S-methylsulfonimidoyl)benzyl)oxy)-1H-pyrazolo[3,4-b]pyridine-1-yl)acetonitrile (Compound 8A), listed below, were prepared according to similar procedures as described herein (m/z means m/z (ES+)(M+H)⁺).

TABLE 8

| Cmpd. | | | | |
|---|---|---|---|---|
| No. | Structure | Name | ¹H NMR | m/z |
| 8A | 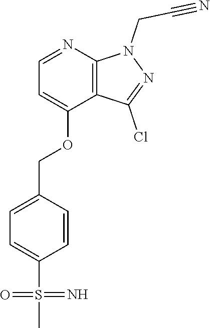 [SMILES: CS(=N)(=O)c1ccc(COc2ccnc3c2c(Cl)nn3CC#N)cc1] | 2-(3-chloro-4-((4-(S- methylsulfonimidoyl)benzyl) oxy)-1H-pyrazolo[3,4-b] pyridine-1-yl)acetonitrile | (300 MHz, DMSO-d6) δ ppm 8.56 (br d, J = 5.47 Hz, 1H), 8.00 (br d, J = 7.99 Hz, 2H), 7.75 (br d, J = 7.81 Hz, 2H), 7.04 (br d, J = 5.30 Hz, 1H), 5.72 (s, 2H), 5.56 (s, 2H), 4.25 (br s, 1H), 3.09 (s, 3H). | 375.95 |

### Example 9

### Synthesis of Compounds 9A-9J

Compounds 9A-9T, listed below, was prepared according to similar procedures as described herein (m/z means m/z (ES+)(M+H)⁺).

TABLE 9

| Cmpd. | | | | |
|---|---|---|---|---|
| No. | Structure | Name | ¹H NMR | m/z |
| 9A | 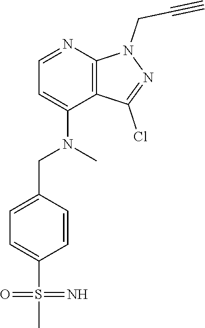 [SMILES: C#CCn1nc(Cl)c2c(N(C)Cc3ccc(S(C)(=N)=O)cc3)ccnc21] | (4-(((3-chloro-1- (prop-2-yn-1-yl)-1H- pyrazolo[3,4-b] pyridine-4-yl) (methyl)amino) methyl)phenyl) (imino)(methyl)-λ⁶- sulfanone | (300 MHz, DMSO-d6) δ ppm 8.25 (br s, 1H), 7.92 (br s, 2H), 7.49 (br s, 2H), 6.59 (br s, 1H), 5.22 (br s, 2H), 4.81 (br s, 2H), 4.20 (br s, 1H), 3.39 (br s, 1H), 3.07 (br s, 3 H), 3.01 (br s, 3H). | 388.00 |
| 9B | 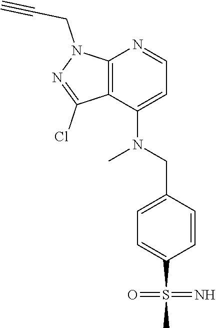 [SMILES: C#CCn1nc(Cl)c2c(N(C)Cc3ccc([S@](C)(=N)=O)cc3)ccnc21] | (R)-(4-(((3-chloro-1- (prop-2-yn-1-yl)-1H- pyrazolo[3,4- b]pyridine-4- yl)(methyl)amino) methyl)phenyl)(imino) (methyl)-λ6-sulfanone | (400 MHz, DMSO-d6) δ 8.25-8.24 (m, 1H), 7.92- 7.90 (m, 2H), 7.50-7.48 (m, 2H), 6.59-6.57 (m, 1H), 5.22-5.21 (m, 2H), 4.80 (s, 2H), 4.20 (s, 1H), 3.38-3.33 (m, 1H), 3.17- 3.00 (m, 6H). | 390.05 |
| 9C | 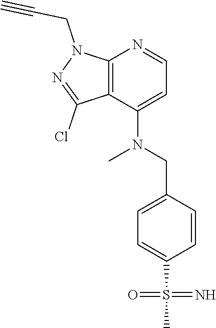 [SMILES: C#CCn1nc(Cl)c2c(N(C)Cc3ccc([S@@](C)(=N)=O)cc3)ccnc21] | (S)-(4-(((3-chloro-1- (prop-2-yn-1-yl)-1H- pyrazolo[3,4- b]pyridine-4- yl)(methyl)amino) methyl)phenyl)(imino) (methyl)-λ6-sulfanone | (300 MHz, DMSO-d6) δ 8.25-8.23 (m, 1H), 7.92- 7.90 (m, 2H), 7.50-7.48 (m, 2H), 6.63-6.57 (m, 1H), 5.21 (s, 2H), 4.80 (s, 2H), 4.15-3.37 (m, 1H), 3.19-3.00 (m, 6H), 1.10- 0.86 (m, 1H). | 388.05 |
| 9D | 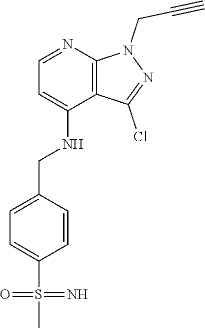 [SMILES: C#CCn1nc(Cl)c2c(NCc3ccc(S(C)(=N)=O)cc3)ccnc21] | (4-(((3-chloro-1- (prop-2-yn-1-yl)-1H- pyrazolo[3,4- b]pyridine-4-yl) amino)methyl) phenyl)(imino) (methyl)-λ⁶-sulfanone | (300 MHz, DMSO-d6) δ ppm 8.03 (d, J = 5.7 Hz, 1H), 7.89 (d, J = 8.4 Hz, 2H), 7.59 (d, J = 8.4 Hz, 2H), 7.45 (m, 1H), 6.14 (d, J = 5.7 Hz, 1H), 5.13 (d, J = 2.4 Hz, 2H), 4.69 (d, J = 6.3 Hz, 2H), 4.15 (s, 1H), 3.35 (m, 1H), 3.04 (s, 3H). | 373.95 |
| 9E | 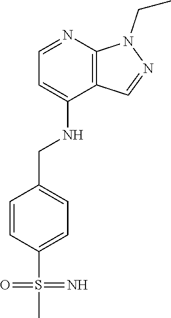 [SMILES: CCn1ncc2c(NCc3ccc(S(C)(=N)=O)cc3)ccnc21] | (4-(((1-ethyl-1H- pyrazolo[3,4- b]pyridine-4-yl) amino)methyl) phenyl)(imino) (methyl)-λ⁶-sulfanone | HCl salt (300 MHz, methanol-d4) δ ppm 8.41 (s, 1H), 8.07 (d, J = 5.7 Hz, 1H), 7.75 (d, J = 6.3 Hz, 2H), 7.66 (d, J = 6.3 Hz, 2H), 6.61 (d, J = 5.7 Hz, 1H), 4.95 (q, J = 5.4 Hz, 2H), 4.86 (s, 2H), 2.81 (s, 3H), 1.53 (t, J = 5.4 Hz, 3H). | 330.05 |
| 9F | 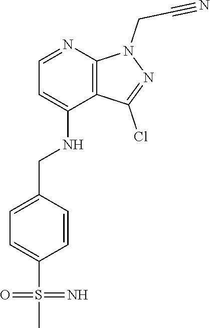 [SMILES: CS(=N)(=O)c1ccc(CNc2ccnc3c2c(Cl)nn3CC#N)cc1] | 2-(3-chloro-4-((4-(S- methylsulfonimidoyl) benzyl)amino)-1H- pyrazolo[3,4-b] pyridine-1-yl) acetonitrile | (300 MHz, DMSO-d6) δ ppm 8.08 (d, J = 5.7 Hz, 1H), 7.89 (d, J = 8.1 Hz, 2H), 7.59 (d, J = 8.1 Hz, 2H), 7.54 (m, 1H), 6.20 (d, J = 5.7 Hz, 1H), 5.58 (s, 2H), 4.70 (d, J = 6.0 Hz, 2H), 4.16 (s, 1H), 3.04 (s, 3H). | 375.00 |
| 9G | 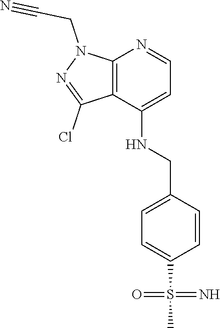 [SMILES: C[S@](=N)(=O)c1ccc(CNc2ccnc3c2c(Cl)nn3CC#N)cc1] | (S)-2-(3-chloro-4-((4- (S- methylsulfonimidoyl) benzyl)amino)-1H- pyrazolo [3,4-b]pyridine-1-yl) acetonitrile | (300 MHz, DMSO-d6) δ ppm 7.88-8.08 (m, 3H), 7.58-7.60 (m, 3H), 6.20 (s, 1H), 5.56 (s, 2H), 4.70 (bs, 2H), 4.15 (s, 1H), 3.03 (s, 3H). | 374.9 |
| 9H | 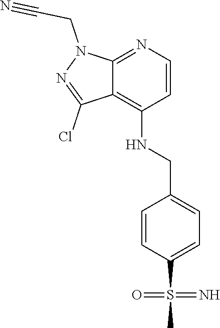 [SMILES: C[S@@](=N)(=O)c1ccc(CNc2ccnc3c2c(Cl)nn3CC#N)cc1] | (R)-2-(3-chloro-4- ((4-(S- methylsulfonimidoyl) benzyl)amino)-1H- pyrazolo[3,4- b]pyridine-1- yl)acetonitrile | (400 MHz, DMSO-d6) δ 8.07 (s, 1H), 7.89-7.79 (m, 2H), 7.59-7.40 (m, 3H), 6.20 (s, 1H), 5.58-5.46 (m, 2H), 4.70-4.61 (m, 2H), 4.24-4.16 (m, 1H), 3.21- 3.04 (m, 3H). | 375.00 |
| 9I | 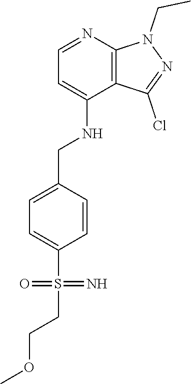 [SMILES: CCn1nc(Cl)c2c(NCc3ccc(S(=N)(=O)CCOC)cc3)ccnc21] | (4-(((3-chloro-1- ethyl-1H-pyrazolo [3,4-b]pyridine-4-yl) amino)methyl) phenyl)(imino)(2- methoxyethyl)-λ⁶- sulfanone | (300 MHz, DMSO-d6) δ ppm 7.99 (d, J = 4.2 Hz, 1H), 7.85 (d, J = 6.3 Hz, 2H), 7.57 (d, 6.0 Hz, 2H), 7.37 (m, 1H), 6.06 (d, J = 4.2 Hz, 1H), 4.67 (d, J = 4.8 Hz, 2H), 4.31 (q, J = 5.4 Hz, 2H), 4.26 (s, 1H), 3.60- 3.50 (m, 2H), 3.41-3.31 (m, 2H), 3.05 (s, 3H), 1.35 (t, J = 5.4 Hz, 3H). | 407.95 |
| 9J | 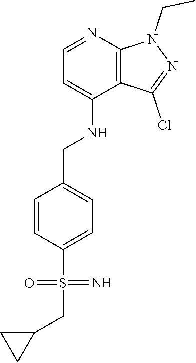 [SMILES: CCn1nc(Cl)c2c(NCc3ccc(S(=N)(=O)CC4CC4)cc3)ccnc21] | (4-(((3-chloro-1- ethyl-1H-pyrazolo [3,4-b]pyridine-4-yl) amino)methyl) phenyl) (cyclopropylmethyl) (imino)-λ⁶-sulfanone | (400 MHz, DMSO-d6) δ ppm 7.98 (d, J = 5.50 Hz, 1 H) 7.86 (d, J = 8.25 Hz, 2 H) 7.57 (d, J = 8.25 Hz, 2 H) 7.37 (t, J = 6.25 Hz, 1 H) 6.04 (d, J = 5.75 Hz, 1 H) 4.68 (d, J = 6.25 Hz, 2 H) 4.31 (q, J = 7.17 Hz, 2 H) 4.11 (s, 1 H) 2.99-3.11 (m, 2 H) 1.35 (t, J = 7.25 Hz, 3 H) 0.79-0.89 (m, 1 H) 0.29-0.38 (m, 2 H) −0.07-−0.01 (m, 2 H) | 404.1 |
| 9K | 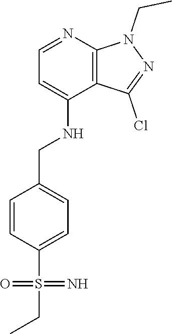 [SMILES: CCn1nc(Cl)c2c(NCc3ccc(S(=N)(=O)CC)cc3)ccnc21] | (4-(((3-chloro-1- ethyl-1H-pyrazolo [3,4-b]pyridine-4-yl) amino)methyl) phenyl)(ethyl) (imino)-λ⁶-sulfanone | (400 MHz, DMSO-d6) δ ppm 8.00 (d, J = 5.50 Hz, 1 H) 7.84 (d, J = 8.25 Hz, 2 H) 7.58 (d, J = 8.25 Hz, 2 H) 7.37 (t, J = 6.25 Hz, 1 H) 6.07 (d, J = 5.50 Hz, 1 H) 4.68 (d, J = 6.25 Hz, 2 H) 4.31 (q, J = 7.09 Hz, 2 H) 4.14 (s, 1 H) 3.09 (q, J = 7.34 Hz, 2 H) 1.35 (t, J = 7.13 Hz, 3 H) 1.04 (t, J = 7.38 Hz, 3 H) | 378.2 |
| 9L | 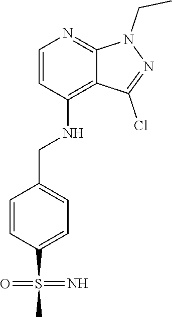 [SMILES: CCn1nc(Cl)c2c(NCc3ccc([S@](C)(=N)=O)cc3)ccnc21] | (R)-(4-(((3-chloro-1- ethyl-1H-pyrazolo [3,4-b]pyridine-4-yl) amino)methyl) phenyl)(imino) (methyl)-λ⁶-sulfanone | (400 MHz, DMSO-d6) δ ppm 8.00 (d, J = 5.75 Hz, 1 H) 7.89 (d, J = 8.25 Hz, 2 H) 7.59 (d, J = 8.25 Hz, 2 H) 7.37 (t, J = 6.25 Hz, 1 H) 6.08 (d, J = 5.50 Hz, 1 H) 4.67 (d, J = 6.25 Hz, 2 H) 4.31 (q, J = 7.09 Hz, 2 H) 4.17 (s, 1 H) 3.04 (s, 3 H) 1.35 (t, J = 7.25 Hz, 3 H) | 364.05 |
| 9M | 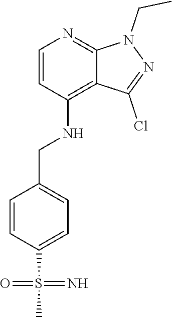 [SMILES: CCn1nc(Cl)c2c(NCc3ccc([S@@](C)(=N)=O)cc3)ccnc21] | (S)-(4-(((3-chloro-1- ethyl-1H-pyrazolo [3,4-b]pyridine-4-yl) amino)methyl) phenyl)(imino) (methyl)-λ⁶-sulfanone | (400 MHz, DMSO-d6) δ ppm 8.00 (d, J = 5.75 Hz, 1 H) 7.89 (d, J = 8.25 Hz, 2 H) 7.59 (d, J = 8.25 Hz, 2 H) 7.37 (t, J = 6.25 Hz, 1 H) 6.08 (d, J = 5.50 Hz, 1 H) 4.67 (d, J = 6.25 Hz, 2 H) 4.31 (q, J = 7.09 Hz, 2 H) 4.17 (s, 1 H) 3.04 (s, 3 H) 1.35 (t, J = 7.25 Hz, 3 H) | 364.05 |
| 9N | 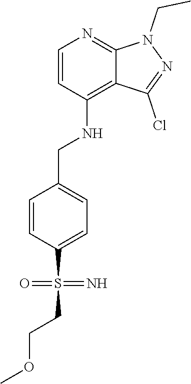 [SMILES: CCn1nc(Cl)c2c(NCc3ccc([S@](=N)(=O)CCOC)cc3)ccnc21] | (R)-(4-(((3-chloro-1- ethyl-1H-pyrazolo [3,4-b]pyridine-4-yl) amino)methyl) phenyl)(imino)(2- methoxyethyl)-λ⁶- sulfanone | (400 MHz, DMSO-d6) δ ppm 8.00 (d, J = 5.75 Hz, 1 H) 7.89 (d, J = 8.25 Hz, 2 H) 7.59 (d, J = 8.25 Hz, 2 H) 7.37 (t, J = 6.25 Hz, 1 H) 6.08 (d, J = 5.50 Hz, 1 H) 4.67 (d, J = 6.25 Hz, 2 H) 4.31 (q, J = 7.09 Hz, 2 H) 4.17 (s, 1 H) 3.04 (s, 3 H) 1.35 (t, J = 7.25 Hz, 3 H) | 408 |
| 9O | 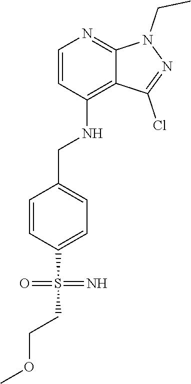 [SMILES: CCn1nc(Cl)c2c(NCc3ccc([S@@](=N)(=O)CCOC)cc3)ccnc21] | (S)-(4-(((3-chloro-1- ethyl-1H- pyrazolo[3,4- b]pyridine-4- yl)amino)methyl) phenyl)(imino)(2- methoxyethyl)-λ6- sulfanone | (400 MHz, DMSO-d6) δ ppm 8.00 (d, J = 5.75 Hz, 1 H) 7.89 (d, J = 8.25 Hz, 2 H) 7.59 (d, J = 8.25 Hz, 2 H) 7.37 (t, J = 6.25 Hz, 1 H) 6.08 (d, J = 5.50 Hz, 1 H) 4.67 (d, J = 6.25 Hz, 2 H) 4.31 (q, J = 7.09 Hz, 2 H) 4.17 (s, 1 H) 3.04 (s, 3 H) 1.35 (t, J = 7.25 Hz, 3 H) | 364.05 |
| 9P | 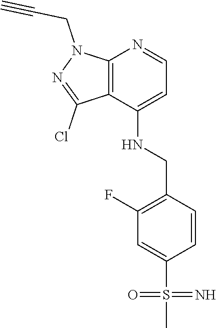 [SMILES: C#CCn1nc(Cl)c2c(NCc3ccc(S(C)(=N)=O)cc3F)ccnc21] | (4-(((3-chloro-1- (prop-2-yn-1-yl)-1H- pyrazolo[3,4- b]pyridine-4- yl)amino)methyl)-3- fluorophenyl)(imino) (methyl)-λ6- sulfanone | (400 MHz, DMSO-d6) δ 8.07-8.06 (m, 1H), 7.75- 7.65 (m, 2H), 7.57-7.56 (m, 1H), 7.33 (s, 1H), 6.18- 6.16 (m, 1H), 5.14 (s, 2H0, 4.72 (s, 2H), 4.33 (s, 1H), 3.36-3.33 (m, 1H), 3.08- 3.00 (m, 3H). | 392.00 |
| 9Q | 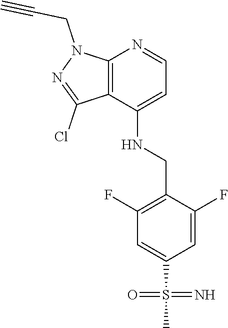 [SMILES: C#CCn1nc(Cl)c2c(NCc3c(F)cc([S@@](C)(=N)=O)cc3F)ccnc21] | (S)-(4-(((3-chloro-1- (prop-2-yn-1-yl)-1H- pyrazolo[3,4- b]pyridine-4- yl)amino)methyl)- 3,5- difluorophenyl) (imino)(methyl)-λ6- sulfanone | (300 MHz, DMSO-d6) δ 8.15-8.13 (m, 1H), 7.65- 7.63 (m, 2H), 7.07 (m, 1H), 6.33-6.32 (m, 1H), 5.12-5.11 (m, 2H), 4.73- 4.71 (m, 2H), 4.50 (s, 1H), 3.35-3.33 (m, 1H), 3.13 (s, 3H). | 410.00 |
| 9R | 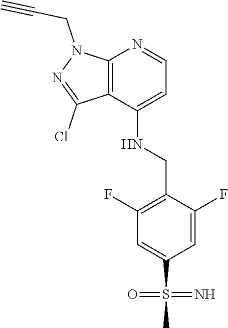 [SMILES: C#CCn1nc(Cl)c2c(NCc3c(F)cc([S@](C)(=N)=O)cc3F)ccnc21] | (R)-(4-(((3-chloro-1- (prop-2-yn-1-yl)-1H- pyrazolo[3,4- b]pyridine-4- yl)amino)methyl)- 3,5- difluorophenyl) (imino)(methyl)-λ6- sulfanone | (300 MHz, DMSO-d6) δ 8.15-8.13 (m, 1H), 7.65- 7.63 (m, 2H), 7.09-7.05 (m, 1H), 6.33-6.32 (m, 1H), 5.12-5.11 (m, 2H), 4.73-4.71 (m, 2H), 4.50 (s, 1H), 3.35-3.33 (m, 1H), 3.12 (s, 3H). | 410.00 |
| 9S | 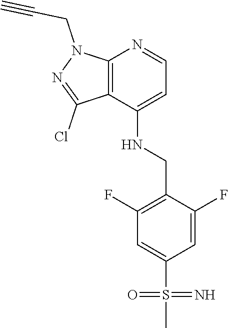 [SMILES: C#CCn1nc(Cl)c2c(NCc3c(F)cc(S(C)(=N)=O)cc3F)ccnc21] | (4-(((3-chloro-1- (prop-2-yn-1-yl)-1H- pyrazolo[3,4- b]pyridine-4- yl)amino)methyl)- 3,5- difluorophenyl) (imino)(methyl)-λ6- sulfanone | (300 MHz, DMSO-d6) δ 8.15-8.13 (m, 1H), 7.65- 7.63 (m, 2H), 7.07-7.00 (m, 1H), 6.33-6.32 (m, 1H), 5.12-5.11 (m, 2H), 4.73-4.71 (m, 1H), 4.50 (s, 1H), 3.35-3.32 (m, 1H), 3.12 (s, 3H). | 409.95 |
| 9T | 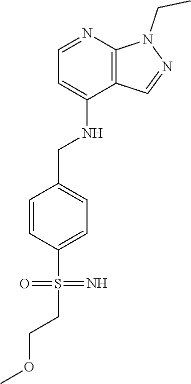 [SMILES: CCn1ncc2c(NCc3ccc(S(=N)(=O)CCOC)cc3)ccnc21] | (4-(((1-ethyl-1H- pyrazolo[3,4- b]pyridine-4- yl)amino)methyl)- phenyl)(imino)(2- methoxyethyl)-λ6- sulfanone | ¹H NMR (300 MHz, DMSO-d₆) δ ppm 8.14 (s, 1 H), 8.05 (t, J = 6.20 Hz, 1 H), 7.96 (d, J = 5.32 Hz, 1 H), 7.86 (d, J = 8.25 Hz, 2 H), 7.56 (d, J = 8.07 Hz, 2 H), 6.08 (d, J = 5.50 Hz, 1 H), 4.64 (d, J = 5.90 Hz, 2 H), 4.34 (q, J = 7.09 Hz, 2 H), 4.26 (s, 1 H), 3.48- 3.66 (m, 2 H), 3.35-3.43 (m, 2 H), 3.05 (s, 3 H), 1.36 (t, J = 7.15 Hz, 3 H) | 374.15 |

### Example 10

### Synthesis of Compounds 10A-10F

Compounds 10A through 10F, listed below, were prepared according to similar procedures as described herein (m/z means m/z (ES+)(M+H)⁺).

TABLE 10

| Cmpd. | | | | |
|---|---|---|---|---|
| No. | Structure | Name | ¹H NMR | m/z |
| 10A | 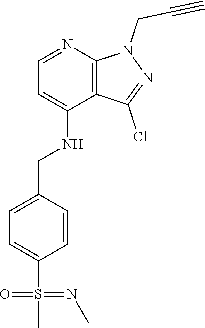 [SMILES: C#CCn1nc(Cl)c2c(NCc3ccc(S(C)(=O)=NC)cc3)ccnc21] | (4-(((3-chloro-1-(prop-2-yn-1-yl)- 1H-pyrazolo[3,4-b]pyridin-4- yl)amino)methyl)phenyl)(methyl) (methylimino)-λ6-sulfanone | (300 MHz, DMSO- d6) δ ppm 8.04 (d, J = 5.7 Hz, 1H), 7.79 (d, J = 7.8 Hz, 2H), 7.61 (d, J = 7.8 Hz, 2H), 7.44 (m, 1H), 6.17 (d, J = 5.4 Hz, 1H), 5.13 (s, 2H), 4.69 (d, J = 6.0 Hz, 2H), 3.35 (s, 1H), 3.08 (s, 3H), 2.44 (s, 3H). | 388.00 |
| 10B | 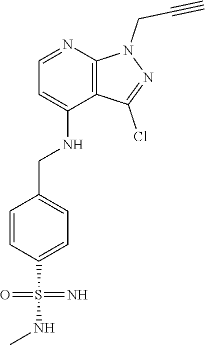 [SMILES: C#CCn1nc(Cl)c2c(NCc3ccc([S@@](=N)(=O)NC)cc3)ccnc21] | (R)-4-(((3-chloro-1-(prop-2-yn-1- yl)-1H-pyrazolo[3,4-b]pyridine- 4-yl)amino)methyl)-N- methylbenzenesulfonimidamide | ¹H NMR (300 MHz, DMSO-d₆) δ ppm 8.03 (d, J = 5.48 Hz, 1 H), 7.80 (d, J = 8.10 Hz, 2 H), 7.52 (d, J = 8.00 Hz, 2 H), 7.42 (t, J = 5.90 Hz, 1 H), 6.66 (br s, 1 H), 6.13 (d, J = 5.74 Hz, 1 H), 5.13 (s, 2 H), 4.66 (d, J = 6.00 Hz, 2 H), 4.09 (br s, 1 H), 3.36 (br s, 1 H), 2.33 (s, 3 H) | 389.00 |
| 10C | 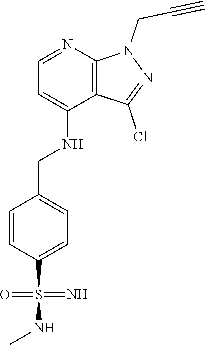 [SMILES: C#CCn1nc(Cl)c2c(NCc3ccc([S@](=N)(=O)NC)cc3)ccnc21] | (S)-4-(((3-chloro-1-(prop-2-yn-1- yl)-1H-pyrazolo[3,4-b]pyridine- 4-yl)amino)methyl)-N- methylbenzenesulfonimidamide | ¹H NMR (300 MHz, DMSO-d₆) δ ppm 8.03 (d, J = 5.30 Hz, 1 H), 7.80 (d, J = 7.60 Hz, 2 H), 7.52 (d, J = 7.90 Hz, 2 H), 7.37-7.48 (m, 1 H), 6.66 (br s, 1 H), 6.13 (d, J = 5.50 Hz, 1 H), 5.13 (br s, 2 H), 4.66 (d, J = 5.50 Hz, 2 H), 4.09 (br s, 1 H), 3.36 (s, 1 H), 2.33 (s, 3 H) | 389.00 |
| 10D | 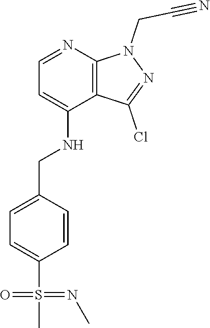 [SMILES: CN=S(C)(=O)c1ccc(CNc2ccnc3c2c(Cl)nn3CC#N)cc1] | 2-(3-chloro-4-((4-(N,S- dimethylsulfonimidoyl)benzyl) amino)-1H-pyrazolo[3,4-b] pyridine-1-yl)acetonitrile | (300 MHz, DMSO- d6) δ ppm 8.08 (d, J = 5.7 Hz, 1H), 7.79 (d, J = 7.5 Hz, 2H), 7.62 (d, J = 7.5 Hz, 2H), 7.56 (m, 1H), 6.24 (d, J = 5.7 Hz, 1H), 5.59 (s, 2H), 4.70 (d, J = 6.3 Hz, 2H), 3.08 (s, 3H), 2.44 (s, 3H). | 389.00 |
| 10E | 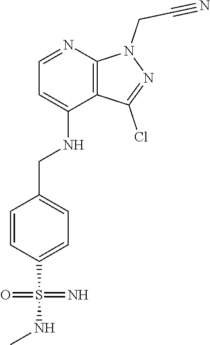 [SMILES: CN[S@](=N)(=O)c1ccc(CNc2ccnc3c2c(Cl)nn3CC#N)cc1] | (S)-4-(((3-chloro-1- (cyanomethyl)-1H-pyrazolo[3,4- b]pyridine-4-yl)amino)methyl)- N- methylbenzenesulfonimidamide | ¹H NMR (300 MHz, DMSO-d₆) δ ppm 8.07 (d, J = 5.57 Hz, 1 H), 7.80 (d, J = 8.08 Hz, 2 H), 7.53 (d, J = 7.80 Hz, 3 H), 6.64 (br s, 1 H), 6.19 (d, J = 5.65 Hz, 1 H), 5.58 (s, 2 H), 4.67 (d, J = 4.40 Hz, 2 H), 4.13 (br s, 1 H), 2.33 (s, 3 H) | 390.05 |
| 10F | 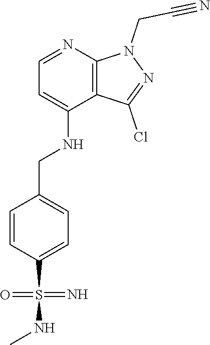 [SMILES: CN[S@@](=N)(=O)c1ccc(CNc2ccnc3c2c(Cl)nn3CC#N)cc1] | (R)-4-(((3-chloro-1- (cyanomethyl)-1H-pyrazolo[3,4- b]pyridine-4-yl)amino)methyl)- N- methylbenzenesulfonimidamide | ¹H NMR (300 MHz, DMSO-d₆) δ ppm 8.07 (d, J = 5.57 Hz, 1 H), 7.80 (d, J = 8.08 Hz, 2 H), 7.53 (d, J = 7.80 Hz, 3 H), 6.64 (br s, 1 H), 6.19 (d, J = 5.65 Hz, 1 H), 5.58 (s, 2 H), 4.67 (d, J = 4.40 Hz, 2 H), 4.13 (br s, 1 H), 2.33 (s, 3H) | 390.00 |

### Example 11

### Synthesis of Compounds 11A-11C

Compounds 11A through 11C, listed below, were prepared according to similar procedures as described herein (m/z means m/z (ES+)(M+H)⁺).

TABLE 11

| Cmpd. | | | | |
|---|---|---|---|---|
| No. | Structure | Name | ¹H NMR | m/z |
| 11A | 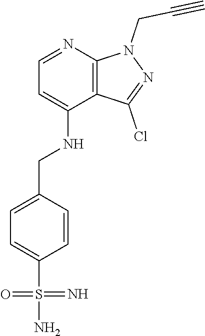 [SMILES: C#CCn1nc(Cl)c2c(NCc3ccc(S(=N)(N)=O)cc3)ccnc21] | 4-(((3-chloro-1-(prop-2-yn-1- yl)-1H-pyrazolo[3,4-b]pyridine- 4-yl)amino)methyl) benzenesulfonimidamide | HCl salt (300 MHz, methanol-d4) δ ppm 9.80- 9.01 (br, 2H), 9.00 (s, 1H), 8.33 (s, 1H), 8.17-8.10 (m, 1H), 8.10-8.01 (m, 2H), 7.80-7.65 (m, 2H), 6.34 (m, 1H), 5.36 (s, 2H), 4.88 (d, J = 6.3 Hz, 2H), 3.48 (s, 1 H). | 375.00 |
| 11B | 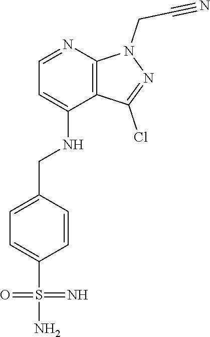 [SMILES: N#CCn1nc(Cl)c2c(NCc3ccc(S(=N)(N)=O)cc3)ccnc21] | 4-(((3-chloro-1-(cyanomethyl)- 1H-pyrazolo[3,4-b]pyridine-4- yl)amino)methyl) benzenesulfonimidamide | (300 MHz, MeOD-d4) δ ppm 8.11-8.18 (m, 3H), 7.80 (d, J = 8.4 Hz, 2H), 6.62 (d, J = 7.2 Hz, 1 H), 5.59 (s, 2H), 5.02 (s, 2H). | 376.00 |
| 11C | 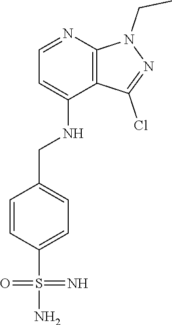 [SMILES: CCn1nc(Cl)c2c(NCc3ccc(S(=N)(N)=O)cc3)ccnc21] | 4-(((3-chloro-1-ethyl-1H- pyrazolo[3,4-b]pyridine-4- yl)amino)methyl) benzenesulfonimidamide | (400 MHz, DMSO-d6) δ ppm 8.13 (d, J = 8.50 Hz, 2 H) 8.11 (d, J = 7.25 Hz, 1 H) 7.80 (d, J = 8.75 Hz, 2 H) 6.60 (d, J = 7.25 Hz, 1 H) 5.04 (s, 2 H) 4.42 (q, J = 7.25 Hz, 2 H) 1.51 (t, J = 7.13 Hz, 3 H) | 365.15 |

### Example 12

### Synthesis of Compounds 12A-12K

Compounds 12A through 12K, listed below, were prepared according to similar procedures as described herein (m/z means m/z (ES+)(M+H)⁺).

TABLE 12

| Cmpd. | | | | |
|---|---|---|---|---|
| No. | Structure | Name | ¹H NMR | m/z |
| 12A | 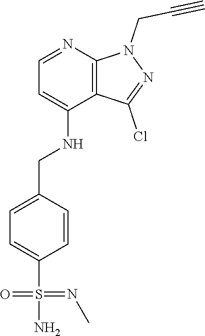 [SMILES: C#CCn1nc(Cl)c2c(NCc3ccc(S(N)(=O)=NC)cc3)ccnc21] | 4-(((3-chloro-1-(prop-2-yn-1-yl)- 1H-pyrazolo[3,4-b]pyridine-4-yl) amino)methyl)-N′-methyl benzenesulfonimidamide | (300 MHz, DMSO-d6) δ ppm 8.03 (d, J = 5.7 1H), 7.80 (d, J = 7.5, 2H), 7.53 (d, J = 7.8 2H), 7.41 (t, J = 5.7 Hz, 1 H), 6.65 (br s, 1H), 6.13 (d, J = 5.4 Hz, 1H), 5.13 (s, 2H), 4.66 (d, J = 6 Hz 2 H), 4.09 (br s, 1 H), 3.34-3.36 (m, 1 H), 2.33 (s, 3H). | 388.95 |
| 12B | 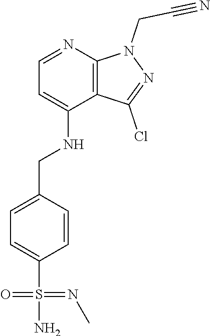 [SMILES: CN=S(N)(=O)c1ccc(CNc2ccnc3c2c(Cl)nn3CC#N)cc1] | 4-(((3-chloro-1-(cyanomethyl)- 1H-pyrazolo[3,4-b]pyridine-4-yl) amino)methyl)-N′-methyl benzenesulfonimidamide | (300 MHz, DMSO-d6) δ ppm 8.07 (d, J = 5.7 1H), 7.80 (d, J = 7.8 Hz, 2H), 7.41-7.55 (m, 3H), 6.65 (br s, 1H), 6.20 (d, J = 5.4 Hz, 1H), 5.64 (s, 2H), 4.67 (d, J = 5.7 Hz 2 H), 4.09 (br s, 1 H), 2.33 (s, 3H). | 390.00 |
| 12C | 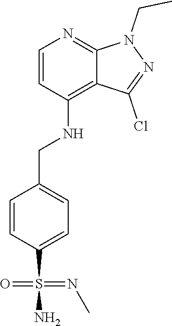 [SMILES: CCn1nc(Cl)c2c(NCc3ccc([S@@](N)(=O)=NC)cc3)ccnc21] | (S)-4-(((3-chloro-1-ethyl-1H- pyrazolo[3,4-b]pyridine-4-yl) amino)methyl)-N′-methyl- benzenesulfonimidamide | (300 MHz, DMSO-d6) δ ppm 8.00 (d, J = 5.50 Hz, 1 H) 7.81 (d, J = 8.07 Hz, 2 H) 7.53 (d, J = 8.07 Hz, 2 H) 7.34 (br t, J = 6.14 Hz, 1 H) 6.08 (d, J = 5.50 Hz, 1 H) 4.65 (br d, J = 6.24 Hz, 2 H) 4.31 (q, J = 7.15 Hz, 2 H) 2.34 (s, 3 H) 1.35 (t, J = 7.24 Hz, 3 H) | 379 |
| 12D | 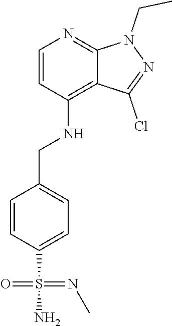 [SMILES: CCn1nc(Cl)c2c(NCc3ccc([S@](N)(=O)=NC)cc3)ccnc21] | (R)-4-(((3-chloro-1-ethyl-1H- pyrazolo[3,4-b]pyridine-4-yl) amino)methyl)-N′-methyl benzenesulfonimidamide | (300 MHz, DMSO-d6) δ ppm 8.00 (d, J = 5.50 Hz, 1 H) 7.81 (d, J = 8.07 Hz, 2 H) 7.53 (d, J = 8.07 Hz, 2 H) 7.34 (br t, J = 6.14 Hz, 1 H) 6.08 (d, J = 5.50 Hz, 1 H) 4.65 (br d, J = 6.24 Hz, 2 H) 4.31 (q, J = 7.15 Hz, 2 H) 2.34 (s, 3 H) 1.35 (t, J = 7.24 Hz, 3 H) | 379 |
| 12E | 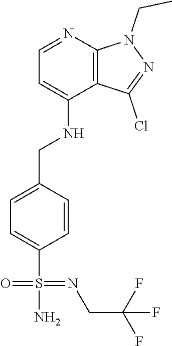 [SMILES: CCn1nc(Cl)c2c(NCc3ccc(S(N)(=O)=NCC(F)(F)F)cc3)ccnc21] | 4-(((3-chloro-1-ethyl-1H- pyrazolo[3,4-b]pyridine-4-yl) amino)methyl)-N′-(2,2,2- trifluoroethyl) benzenesulfonimidamide | (400 MHz, DMSO-d6) δ ppm 8.00 (d, J = 5.50 Hz, 1 H), 7.84 (d, J = 8.25 Hz, 2 H), 7.55 (d, J = 8.25 Hz, 2 H), 7.35 (t, J = 6.13 Hz, 1 H), 6.83- 7.23 (m, 2 H), 6.05 (d, J = 5.50 Hz, 1 H), 4.65 (d, J = 6.00 Hz, 2 H), 4.31 (q, J = 7.25 Hz, 2 H), 3.57 (q, J = 9.76 Hz, 2 H), 1.35 (t, J = 7.13 Hz, 3 H) | 447 |
| 12F | 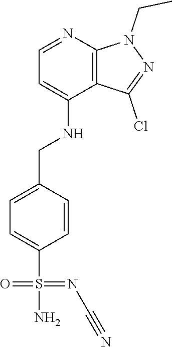 [SMILES: CCn1nc(Cl)c2c(NCc3ccc(S(N)(=O)=NC#N)cc3)ccnc21] | 4-(((3-chloro-1-ethyl-1H- pyrazolo[3,4-b]pyridine-4-yl) amino)methyl)-N′-cyano benzenesulfonimidamide | (400 MHz, DMSO-d6) δ ppm 7.77-8.14 (m, 5 H) 7.63 (br d, J = 7.63 Hz, 2 H) 7.38 (br t, J = 5.54 Hz, 1 H) 6.05 (br d, J = 5.48 Hz, 1 H) 4.69 (br d, J = 5.48 Hz, 2 H) 4.31 (q, J = 6.79 Hz, 2 H) 1.35 (br t, J = 6.97 Hz, 3 H) | 390 |
| 12G | 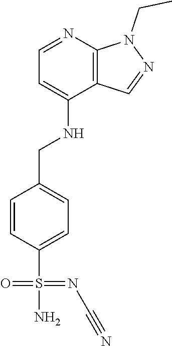 [SMILES: CCn1ncc2c(NCc3ccc(S(N)(=O)=NC#N)cc3)ccnc21] | N′-cyano-4-(((1-ethyl-1H- pyrazolo[3,4-b]pyridine-4- yl)amino)methyl) benzenesulfonimidamide | ¹H NMR (400 MHz, DMSO-d₆) δ ppm 8.15 (s, 1 H), 8.00 (t, J = 6.00 Hz, 1 H), 7.97 (d, J = 5.38 Hz, 1 H), 7.78 (d, J = 8.31 Hz, 2 H), 7.47 (d, J = 8.31 Hz, 2 H), 6.34 (br s, 2 H), 6.09 (d, J = 5.38 Hz, 1 H), 4.58 (d, J = 5.60 Hz, 2 H), 4.34 (q, J = 7.34 Hz, 2 H), 1.36 (t, J = 7.21 Hz, 3 H) | 356.05 |
| 12H | 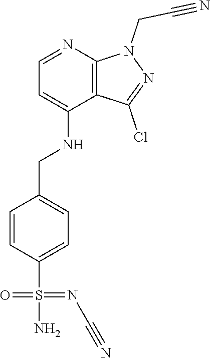 [SMILES: N#CCn1nc(Cl)c2c(NCc3ccc(S(N)(=O)=NC#N)cc3)ccnc21] | 4-(((3-chloro-1-(cyanomethyl)- 1H-pyrazolo[3,4-b]pyridine-4- yl)amino)methyl)-N′- cyanobenzenesulfonimidamide | ¹H NMR (300 MHz, DMSO-d₆) δ ppm 8.07 (d, J = 5.48 Hz, 1 H), 8.00 (br s, 2 H), 7.87 (d, J = 7.99 Hz, 2 H), 7.63 (d, J = 8.00 Hz, 2 H), 7.57 (t, J = 6.20 Hz, 1 H), 6.18 (d, J = 5.65 Hz, 1 H), 5.58 (s, 2 H), 4.71 (d, J = 6.10 Hz, 2 H) | 400.94 |
| 12I | 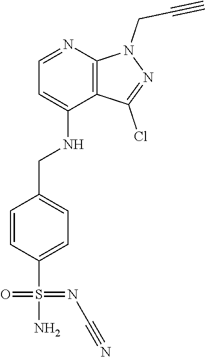 [SMILES: C#CCn1nc(Cl)c2c(NCc3ccc(S(N)(=O)=NC#N)cc3)ccnc21] | 4-(((3-chloro-1-(prop-2-yn-1-yl)- 1H-pyrazolo[3,4-b]pyridine-4- yl)amino)methyl)-N′- cyanobenzenesulfonimidamide | ¹H NMR (300 MHz, DMSO-d₆) δ ppm 8.03 (d, J = 5.56 Hz, 1 H), 7.85 (d, J = 7.90 Hz, 2 H), 7.59 (d, J = 8.08 Hz, 2 H), 7.18- 7.50 (m, 3 H), 6.11 (d, J = 5.57 Hz, 1 H), 5.13 (s, 2 H), 4.68 (d, J = 6.10 Hz, 2 H), 3.36 (s, 1 H) | 400.00 |
| 12J | 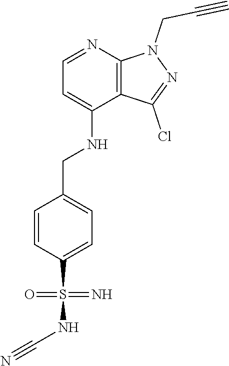 [SMILES: C#CCn1nc(Cl)c2c(NCc3ccc([S@](=N)(=O)NC#N)cc3)ccnc21] | (R)-4-(((3-chloro-1-(prop-2-yn-1- yl)-1H-pyrazolo[3,4-b]pyridine- 4-yl)amino)methyl)-N- cyanobenzenesulfonimidamide | ¹H NMR (300 MHz, DMSO-d₆) δ ppm 8.77 (s, 2 H), 8.03 (d, J = 5.56 Hz, 1 H), 7.85 (d, J = 7.90 Hz, 2 H), 7.59 (d, J = 8.08 Hz, 2 H), 7.18-7.50 (m, 1 H), 6.11 (d, J = 5.57 Hz, 1 H), 5.13 (s, 2 H), 4.68 (d, J = 6.10 Hz, 2 H), 3.36 (s, 1 H) | 400.10 |
| 12K | 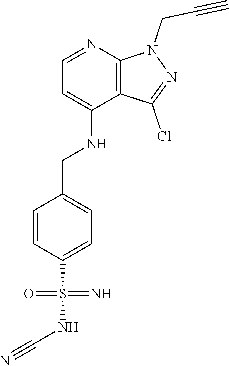 [SMILES: C#CCn1nc(Cl)c2c(NCc3ccc([S@@](=N)(=O)NC#N)cc3)ccnc21] | (S)-4-(((3-chloro-1-(prop-2-yn-1- yl)-1H-pyrazolo[3,4-b]pyridine- 4-yl)amino)methyl)-N- cyanobenzenesulfonimidamide | ¹H NMR (300 MHz, DMSO-d₆) δ ppm 8.77 (s, 2 H), 8.03 (d, J = 5.56 Hz, 1 H), 7.85 (d, J = 7.90 Hz, 2 H), 7.59 (d, J = 8.08 Hz, 2 H), 7.18-7.50 (m, 1 H), 6.11 (d, J = 5.57 Hz, 1 H), 5.13 (s, 2 H), 4.68 (d, J = 6.10 Hz, 2 H), 3.36 (s, 1 H) | 400.00 |

### Example 13

### Synthesis of Compounds 13A-13H

Compounds 13A through 13H, listed below, were prepared according to similar procedures as described herein (m/z means m/z (ES+)(M+H)⁺)

TABLE 13

| Cmpd. | | | | |
|---|---|---|---|---|
| No. | Structure | Name | ¹H NMR | m/z |
| 13A | 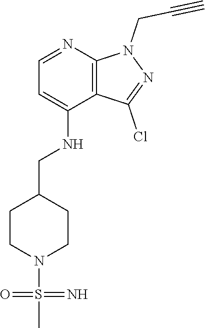 [SMILES: C#CCn1nc(Cl)c2c(NCC3CCN(S(C)(=N)=O)CC3)ccnc21] | 3-chloro-N-((1-(S- methylsulfonimidoyl)piperidin-4-yl) methyl)-1-(prop-2-yn-1-yl)-1H- pyrazolo[3,4-b]pyridine-4-amine | (300 MHz, DMSO-d6) δ ppm 8.11 (d, J = 5.4, 1H), 6.67-6.72 (m, 1H), 6.38 (d, J = 5.7 Hz, 1H), 5.75 (br s, 1H), 5.13 (s, 2H), 3.64-3.74 (m, 2H), 3.36 (s, 1H), 3.26-3.30 (m, 2H), 2.92 (s, 3H), 2.62-2.73 (m, 2H), 1.80- 1.85 (m, 3H), 1.26-1.30 (m, 2H). | 381.05 |
| 13B | 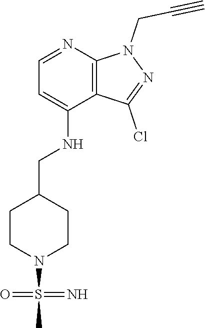 [SMILES: C#CCn1nc(Cl)c2c(NCC3CCN([S@](C)(=N)=O)CC3)ccnc21] | (S)-3-chloro-N-((1-(S- methylsulfonimidoyl)piperidin-4-yl) methyl)-1-(prop-2-yn-1-yl)-1H- pyrazolo[3,4-b]pyridine-4-amine | (300 MHz, DMSO-d6) δ ppm 8.1 (d, J = 5.7, 1H), 6.63-6.71 (m, 1H), 6.37 (d, J = 5.7 Hz, 1H), 5.10-5.13 (m, 2H), 4.05 (br s, 1H), 3.62-3.73 (m, 2H), 3.35-3.37 (m, 1H), 3.25-3.29 (m, 2H), 2.77 (s, 3H), 2.54-2.65 (m, 2H), 1.79-1.83 (m, 3H), 1.22-1.27 (m, 2H). | 381.15 |
| 13C | 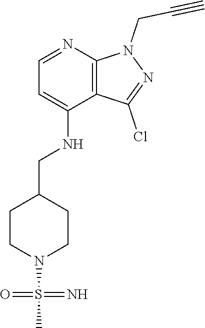 [SMILES: C#CCn1nc(Cl)c2c(NCC3CCN([S@@](C)(=N)=O)CC3)ccnc21] | (R)-3-chloro-N-((1-(S- methylsulfonimidoyl)piperidin-4- yl)methyl)-1-(prop-2-yn-1-yl)-1H- pyrazolo[3,4-b]pyridine-4-amine | (300 MHz, DMSO-d6) δ ppm 8.1 (d, J = 5.7, 1H), 6.63-6.71 (m, 1H), 6.37 (d, J = 5.7 Hz, 1H), 5.10-5.13 (m, 2H), 4.05 (br s, 1H), 3.62-3.75 (m, 2H), 3.35-3.37 (m, 1H), 3.25-3.29 (m, 2H), 2.77 (s, 3H), 2.54-2.65 (m, 2H), 1.79-1.83 (m, 3H), 1.22-1.27 (m, 2H). | 381.05 |
| 13D | 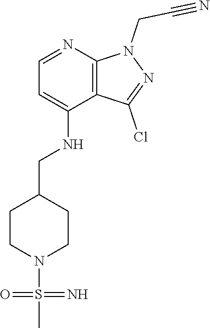 [SMILES: CS(=N)(=O)N1CCC(CNc2ccnc3c2c(Cl)nn3CC#N)CC1] | 2-(3-chloro-4-(((1-(S- methylsulfonimidoyl)piperidin-4-yl) methyl)amino)-1H-pyrazolo[3,4-b] pyridine-1-yl)acetonitrile | (300 MHz, DMSO-d6) δ ppm 8.13 (d, J = 5.7, 1H), 6.78 (t, J = 5.7 Hz, 1H), 6.43 (d, J = 5.7 Hz, 1 H), 5.57 (s, 2H), 3.61-3.72 (m, 2H), 3.48 (s, 1H), 3.26-3.30 (m, 2H), 2.69 (s, 3H), 2.5-2.61 (m, 2H), 1.77-1.82 (m, 3H), 1.21- 1.25 (m, 2H). | 382.0 |
| 13E | 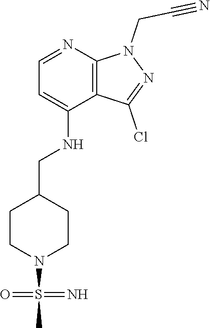 [SMILES: C[S@@](=N)(=O)N1CCC(CNc2ccnc3c2c(Cl)nn3CC#N)CC1] | (S)-2-(3-chloro-4-(((1-(S- methylsulfonimidoyl)piperidin-4-yl) methyl)amino)-1H-pyrazolo[3,4-b] pyridine-1-yl)acetonitrile | (300 MHz, DMSO-d6) δ ppm 8.14 (d, J = 5.7, 1H), 6.77 (t, J = 5.7 Hz, 1H), 6.43 (d, J = 5.7 Hz, 1 H), 5.57 (s, 2H), 3.62-3.73 (m, 2H), 3.48 (s, 1H), 3.26-3.30 (m, 2H), 2.69 (s, 3H), 2.50-2.61 (m, 2H), 1.78-1.82 (m, 3H), 1.22-1.26 (m, 2H). | 382.00 |
| 13F | 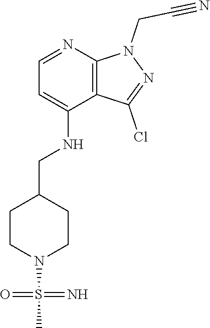 [SMILES: C[S@](=N)(=O)N1CCC(CNc2ccnc3c2c(Cl)nn3CC#N)CC1] | (R)-2-(3-chloro-4-(((1-(S- methylsulfonimidoyl)piperidin-4- yl)methyl)amino)-1H-pyrazolo[3,4- b]pyridine-1-yl)acetonitrile | (300 MHz, DMSO-d6) δ ppm 8.14 (d, J = 5.7, 1H), 6.77 (t, J = 5.7 Hz, 1H), 6.43 (d, J = 5.7 Hz, 1 H), 5.57 (s, 2H), 3.62-3.73 (m, 2H), 3.48 (s, 1H), 3.26-3.30 (m, 2H), 2.69 (s, 3H), 2.50-2.61 (m, 2H), 1.78-1.82 (m, 3H), 1.22-1.26 (m, 2H). | 382.00 |
| 13G | 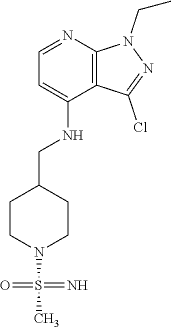 [SMILES: CCn1nc(Cl)c2c(NCC3CCN([S@@](C)(=N)=O)CC3)ccnc21] | (R)-3-chloro-1-ethyl-N-((1-(S- methylsulfonimidoyl)piperidin-4-yl) methyl)-1H-pyrazolo[3,4-b]pyridine- 4-amine | (300 MHz, DMSO-d6) δ ppm 8.07 (d, J = 5.69 Hz, 1 H) 6.57 (br t, J = 5.69 Hz, 1 H) 6.30 (d, J = 5.69 Hz, 1 H) 4.30 (q, J = 7.27 Hz, 2 H) 3.58-3.77 (m, 2 H) 3.48 (d, J = 0.92 Hz, 1 H) 3.26 (br t, J = 6.42 Hz, 2 H) 2.69 (d, J = 1.47 Hz, 3 H) 2.54-2.63 (m, 2 H) 1.74-1.87 (m, 3 H) 1.35 (t, J = 7.24 Hz, 3 H) 1.17- 1.30 (m, 2 H) | 371 |
| 13H | 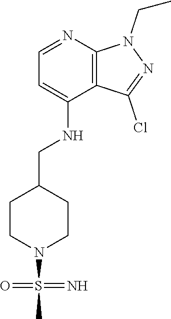 [SMILES: CCn1nc(Cl)c2c(NCC3CCN([S@](C)(=N)=O)CC3)ccnc21] | (S)-3-chloro-1-ethyl-N-((1-(S- methylsulfonimidoyl)piperidin-4- yl)methyl)-1H-pyrazolo[3,4- b]pyridine-4-amine | (300 MHz, DMSO-d6) δ ppm 8.07 (d, J = 5.69 Hz, 1 H) 6.57 (t, J = 5.69 Hz, 1 H) 6.30 (d, J = 5.69 Hz, 1 H) 4.30 (q, J = 7.27 Hz, 2 H) 3.58-3.77 (m, 2 H) 3.48 (d, J = 0.92 Hz, 1 H) 3.26 (t, J = 6.42 Hz, 2 H) 2.69 (d, J = 1.47 Hz, 3 H) 2.54-2.63 (m, 2 H) 1.74- 1.87 (m 3 H) 1.35 (t, J = 7.24 Hz, 3 H) 1.17- 1.30 (m, 2 H) | 371.00 |

### Example 14

### Synthesis of Compounds 14A-14K

Compounds 14A through 14K, listed below, were prepared according to similar procedures as described herein (m/z means m/z (ES+)(M+H)⁺).

TABLE 14

| Cmpd. | | | | |
|---|---|---|---|---|
| No. | Structure | Name | ¹H NMR | m/z |
| 14A | 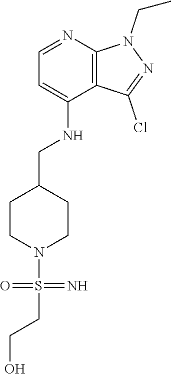 [SMILES: CCn1nc(Cl)c2c(NCC3CCN(S(=N)(=O)CCO)CC3)ccnc21] | 2-(4-(((3-chloro-1-ethyl- 1H-pyrazolo[3,4-b] pyridine-4-yl)amino) methyl)piperidine-1- sulfonimidoyl)ethan-1-ol | (300 MHz, DMSO-d6) δ ppm 8.06 (d, J = 5.4 Hz, 1H), 6.54 (m, 1H), 6.29 (d, J = 5.7 Hz, 1H), 4.90-4.94 (m, 1H), 4.31 (q, J = 6.9 Hz, 2H), 3.33-3.79 (m, 5H), 3.22-3.27 (m, 2H), 2.89-3.15 (m, 2H), 2.51-2.71 (m, 2H), 1.70-1.90 (m, 3H), 1.35 (t, J = 6.9 Hz, 3H), 1.11-1.35 (m, 2H). | 401.10 |
| 14B | 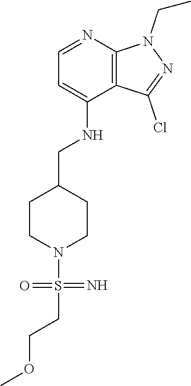 [SMILES: CCn1nc(Cl)c2c(NCC3CCN(S(=N)(=O)CCOC)CC3)ccnc21] | 3-chloro-1-ethyl-N-((1-(2- methoxyethylsulfonimidoyl) piperidin-4-yl)methyl)- 1H-pyrazolo[3,4-b] pyridine-4-amine | (300 MHz, DMSO-d6) δ ppm 8.07 (d, J = 5.7 Hz, 1H), 6.53-6.57 (m, 1H), 6.29 (d, J = 5.4 Hz, 1H), 4.32 (q, J = 7.2 Hz, 2H), 3.63-3.73 (m, 5H), 3.23- 3.30 (m, 5H), 3.03-3.19 (m, 2H), 2.51-2.70 (m, 2H), 2.70-2.85 (m, 3H), 1.37 (t, J = 7.2 Hz, 3H), 1.12-1.30 (m, 3H). | 415.05 |
| 14C | 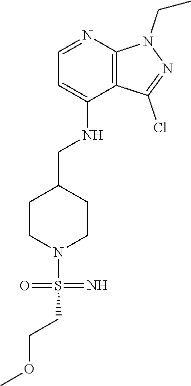 [SMILES: CCn1nc(Cl)c2c(NCC3CCN([S@@](=N)(=O)CCOC)CC3)ccnc21] | (R)-3-chloro-1-ethyl-N- ((1-(2- methoxyethylsulfonimidoyl) piperidin-4-yl)methyl)- 1H-pyrazolo[3,4-b] pyridine-4-amine | (300 MHz, DMSO-d6) δ ppm 8.07 (d, J = 5.56 Hz, 1 H) 6.54 (br t, J = 5.97 Hz, 1 H) 6.29 (d, J = 5.65 Hz, 1 H) 4.30 (q, J = 7.18 Hz, 2 H) 3.54-3.77 (m, 5 H) 3.31 (s, 2 H) 3.21-3.29 (m, 3 H) 2.99-3.20 (m, 2 H) 2.57-2.77 (m 2 H) 1.77 (br d, J = 10.68 Hz, 3 H) 1.34 (t, J = 7.18 Hz, 3 H) 1.13-1.29 (m, 2 H) | 415.2 |
| 14D | 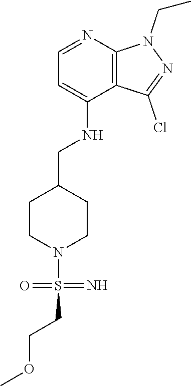 [SMILES: CCn1nc(Cl)c2c(NCC3CCN([S@](=N)(=O)CCOC)CC3)ccnc21] | (S)-3-chloro-1-ethyl-N- ((1-(2- methoxyethylsulfonimidoyl) piperidin-4-yl)methyl)- 1H-pyrzolo[3,4-b] pyridine-4-amine | (300 MHz, DMSO-d6) δ ppm 8.07 (d, J = 5.65 Hz, 1 H) 6.54 (br t, J = 5.97 Hz, 1 H) 6.29 (d, J = 5.65 Hz, 1 H) 4.30 (q, J = 7.21 Hz, 2 H) 3.55-3.77 (m, 5 H) 3.31 (br s, 1 H) 3.25 (s, 4 H) 3.01-3.19 (m, 2 H) 2.57-2.75 (m, 2 H) 1.77 (br d, J = 10.68 Hz, 3 H) 1.34 (t, J = 7.18 Hz, 3 H) 1.14-1.28 (m, 2 H) | 415.2 |
| 14E | 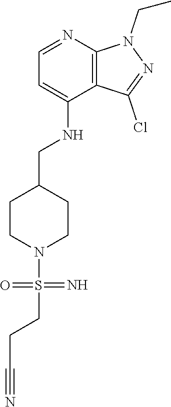 [SMILES: CCn1nc(Cl)c2c(NCC3CCN(S(=N)(=O)CCC#N)CC3)ccnc21] | 3-(4-(((3-chloro-1-ethyl- 1H-pyrazolo[3,4-b] pyridine-4-yl)amino) methyl)piperidine-1- sulfonimidoyl) propanenitrile | (300 MHz, DMSO-d6) δ ppm 8.07 (d, J = 5.69 Hz, 1 H), 6.57 (t, J = 5.90 Hz, 1 H), 6.30 (d, J = 5.69 Hz, 1 H), 4.30 (q, J = 7.27 Hz, 2 H), 3.92 (s, 1 H), 3.69 (t, J = 13.20 Hz, 2 H), 3.14- 3.30 (m, 4 H), 2.83-2.94 (m, 2 H), 2.71 (q, J = 10.09 Hz, 2 H), 1.69- 1.89 (m, 3 H), 1.35 (t, J = 7.15 Hz, 3 H), 1.13-1.28 (m, 2 H) | 410.1 |
| 14F | 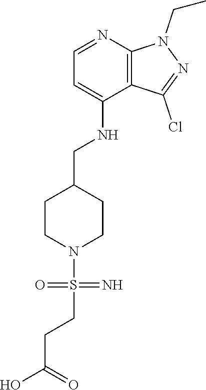 [SMILES: CCn1nc(Cl)c2c(NCC3CCN(S(=N)(=O)CCC(=O)O)CC3)ccnc21] | 3-(4-(((3-chloro-1-ethyl- 1H-pyrazolo[3,4-b] pyridine-4-yl)amino) methyl)piperidine-1- sulfonimidoyl)propanoic acid | (300 MHz, DMSO-d6) δ ppm 12.43 (br s, 1 H), 8.07 (d, J = 5.69 Hz, 1 H), 6.56 (t, J = 6.10 Hz, 1 H), 6.30 (d, J = 5.69 Hz, 1 H), 4.30 (q, J = 7.15 Hz, 2 H), 3.61-3.77 (m, 2 H), 3.21- 3.29 (m, 2 H), 2.99-3.16 (m, 2 H), 2.57-2.75 (m, 4 H), 1.67-1.91 (m, 3 H), 1.35 (t, J = 7.24 Hz, 3 H), 1.11- 1.30 (m, 2 H) | 429.1 |
| 14G | 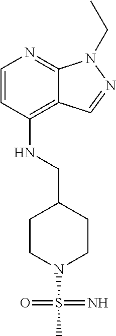 [SMILES: CCn1ncc2c(NCC3CCN([S@@](C)(=N)=O)CC3)ccnc21] | (R)-1-ethyl-N-((1-(S- methylsulfonimidoyl) piperidin-4-yl)methyl)-1H- pyrazolo[3,4-b]pyridine-4- amine | (300 MHz, DMSO-d6) δ ppm 8.10 (s, 1 H), 8.00 (m, 1 H), 7.35 (m, 1H), 6.15 (m, 1H), 4.30-4.35 (m, 2H), 3.60-3.70 (m, 2H), 3.45 (m, 1H), 3.15-3.20 (m, 2H), 2.70 (s, 3H), 2.50-2.60 (m, 2H), 1.85-1.90 (m, 2H), 1.70 (m, 1H), 1.30-1.40 (m, 3H), 1.10-1.30 (m, 2H) | 337.1 |
| 14H | 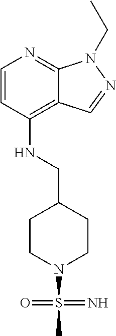 [SMILES: CCn1ncc2c(NCC3CCN([S@](C)(=N)=O)CC3)ccnc21] | (S)-1-ethyl-N-((1-(S- methylsulfonimidoyl) piperidin-4-yl)methyl)-1H- pyrazolo[3,4-b]pyridine-4- amine | (300 MHz, DMSO-d6) δ ppm 8.10 (s, 1 H), 8.00 (m, 1 H), 7.35 (m, 1H), 6.15 (m, 1H), 4.30-4.35 (m, 2H), 3.60-3.70 (m, 2H), 3.45 (m, 1H), 3.15-3.20 (m, 2H), 2.70 (s, 3H), 2.50-2.60 (m, 2H), 1.85-1.90 (m, 2H), 1.70 (m, 1H), 1.30-1.40 (m, 3H), 1.10-1.30 (m, 2H) | 337.1 |
| 14I | 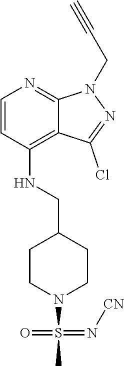 [SMILES: C#CCn1nc(Cl)c2c(NCC3CCN([S@@](C)(=O)=NC#N)CC3)ccnc21] | (S)-N-((4-(((3-chloro-1- (prop-2-yn-1-yl)-1H- pyrazolo[3,4-b]pyridine-4- yl)amino)methyl)piperidin- 1-yl)(methyl)(oxo)-λ6- sulfaneylidene)cyanamide | (300 MHz, DMSO-d6) δ ppm 8.10 (m, 1H), 6.70 (m, 1H), 6.40 (m, 1H), 5.10 (s, 2H), 3.60-3.70 (m, 2H), 3.20-3.40 (m, 6H), 2.80-2.95 (m, 2H), 1.80-2.00 (m, 3H), 1.20-1.40 (m, 2H) | 406.1 |
| 14J | 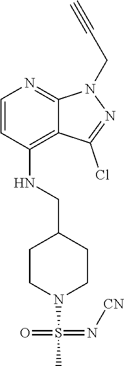 [SMILES: C#CCn1nc(Cl)c2c(NCC3CCN([S@](C)(=O)=NC#N)CC3)ccnc21] | (R)-N-((4-(((3-chloro-1- (prop-2-yn-1-yl)-1H- pyrazolo[3,4-b]pyridine-4- yl)amino)methyl)piperidin- 1-yl)(methyl)(oxo)-λ6- sulfaneylidene)cyanamide | (300 MHz, DMSO-d6) δ ppm 8.10 (m, 1H), 6.70 (m, 1H), 6.40 (m, 1H), 5.10 (s, 2H), 3.60-3.70 (m, 2H), 3.20-3.40 (m, 6H), 2.80-2.95 (m, 2H), 1.80-2.00 (m, 3H), 1.20-1.40 (m, 2H) | 406.1 |
| 14K | 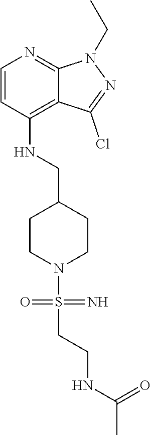 [SMILES: CCn1nc(Cl)c2c(NCC3CCN(S(=N)(=O)CCNC(C)=O)CC3)ccnc21] | N-(2-(4-(((3-chloro-1- ethyl-1H-pyrazolo[3,4- b]pyridine-4- yl)amino)methyl)piperidine- 1- sulfonimidoyl)ethyl) acetamide | (300 MHz, DMSO-d6) δ ppm 8.10 (m, 1H), 7.90-8.00 (m, 1H), 6.50 (m, 1H), 6.30 (m, 1H), 4.20-4.35 (m, 2H), 3.60-3.80 (m, 3H), 3.20- 3.40 (m, 4H), 2.80-3.00 (m, 2H), 2.60-2.70 (m, 2H), 1.70-1.85 (m, 6H), 1.10-1.40 (m, 5H) | 406.1 |

### Example 15

### Synthesis of Compounds 15A-15I

Compounds 15A through 15I, listed below, were prepared according to similar procedures as described herein (m/z means m/z (ES+)(M+H)⁺)

TABLE 15

| Cmpd. | | | | |
|---|---|---|---|---|
| No. | Structure | Name | ¹H NMR | m/z |
| 15A | 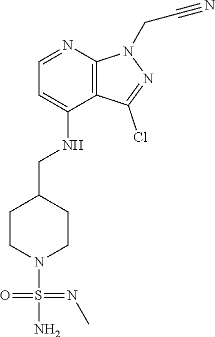 [SMILES: CN=S(N)(=O)N1CCC(CNc2ccnc3c2c(Cl)nn3CC#N)CC1] | 4-(((3-chloro-1- (cyanomethyl)-1H-pyrazolo [3,4-b]pyridine-4-yl)amino) methyl)-N′- methylpiperidine-1- sulfonimidamide | HCl salt (300 MHz, DMSO- d6) δ ppm 9.10-9.45 (m, 3H), 8.20 (d, J = 6.3 Hz, 1H), 7.90 (bs, 1H), 7.39 (bs, 1H), 6.61 (d, J = 6.3 Hz, 1H), 5.73 (s, 2H), 3.69-3.73 (m, 2H), 3.37-3.41 (m, 2H), 2.98-3.05 (m, 2H), 2.66 (s, 3H), 1.83- 1.87 (m, 3H), 1.24-1.35 (m, 2H). | 397.1 |
| 15B | 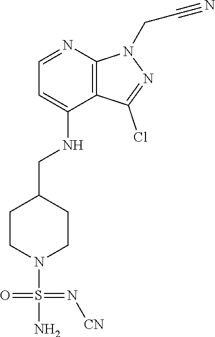 [SMILES: N#CCn1nc(Cl)c2c(NCC3CCN(S(N)(=O)=NC#N)CC3)ccnc21] | 4-(((3-chloro-1- (cyanomethyl)-1H-pyrazolo [3,4-b]pyridine-4-yl)amino) methyl)-N′- cyanopiperidine-1- sulfonimidamide | (300 MHz, DMSO-d6) δ ppm 8.13 (d, J = 5.7 Hz, 1H), 7.99 (bs, 2H), 6.78-6.82 (m, 1H), 6.43 (d, J = 6.0 Hz, 1H), 5.57 (s, 2H), 3.61-3.64 (m, 2H), 3.26-3.30 (m, 2H), 2.68-2.72 (m, 2H), 1.80-1.92 (m, 3H), 1.20-1.40 (m, 2H). | 408.0 |
| 15C | 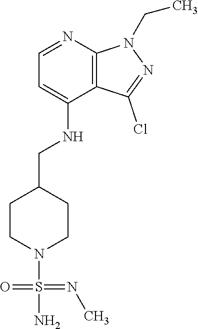 [SMILES: CCn1nc(Cl)c2c(NCC3CCN(S(N)(=O)=NC)CC3)ccnc21] | 4-(((3-chloro-1-ethyl-1H- pyrazolo[3,4-b]pyridine-4- yl)amino)methyl)-N′- methylpiperidine-1- sulfonimidamide | (400 MHz, DMSO-d6) δ ppm 9.15-9.61 (m, 2 H) 7.90-8.31 (m, 2 H) 6.68- 6.83 (m, 1 H) 4.42-4.59 (m, 2 H) 3.72 (br d, J = 12.26 Hz, 2 H) 3.49 (br d, J = 5.75 Hz, 2 H) 3.02 (br s, 2 H) 2.66 (s, 3 H) 1.75-1.99 (m, 3 H) 1.38 (t, J = 7.13 Hz, 5 H). 1H NMR (400 MHz, METHANOL-d4) δ ppm 8.14 (d, J = 7.25 Hz, 1 H) 6.84 (br dd, J = 6.25, 4.00 Hz, 1 H) 4.40 (qd, J = 7.00, 2.75 Hz, 2 H) 3.88 (br d, J = 5.75 Hz, 2 H) 3.60 (br d, J = 6.00 Hz, 2 H) 3.06 (br t, J = 10.76 Hz, 2 H) 2.81 (s, 3 H) 1.94- 2.12 (m, 3 H) 1.50 (t, J = 7.25 Hz, 5 H) | 386.1 |
| 15D | 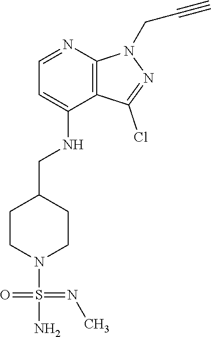 [SMILES: C#CCn1nc(Cl)c2c(NCC3CCN(S(N)(=O)=NC)CC3)ccnc21] | 4-(((3-chloro-1-(prop-2-yn- 1-yl)-1H-pyrazolo[3,4- b]pyridine-4- yl)amino)methyl)-N′- methylpiperidine-1- sulfonamidamide | (400 MHz, DMSO-d6) δ ppm 9.00 (br s, 2H), 8.10 (m, 1H), 6.80 (m, 1H), 6.40 (m, 1H), 5.15 (s, 2H), 4.60- 4.75 (m, 2H), 3.40 (m, 1H), 3.20-3.30 (m, 2H), 2.85- 3.00 (m, 2H), 2.70 (s, 3H), 1.80-2.00 (m, 3H), 1.20- 1.40 (m, 2H) | 396.0 |
| 15E | 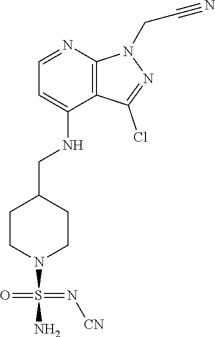 [SMILES: N#CCn1nc(Cl)c2c(NCC3CCN([S@@](N)(=O)=NC#N)CC3)ccnc21] | (S)-4-(((3-chloro-1- (cyanomethyl)-1H- pyrazolo[3,4-b]pyridine-4- yl)amino)methyl)-N′- cyanopiperidine-1- sulfonimidamide | (400 MHz, DMSO-d6) δ ppm 8.15 (m, 1H), 8.00 (br s, 2H), 6.80 (m, 1H), 6.45 (m, 1H), 5.55 (s, 2H), 3.60- 3.70 (m, 2H), 3.20-3.40 (m, 2H), 2.60-2.75 (m, 2H), 1.70-1.90 (m, 3H), 1.20-1.40 (m, 2H) | 408.1 |
| 15F | 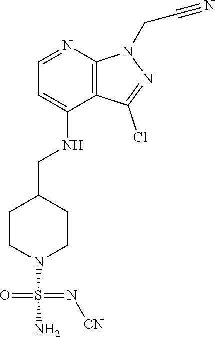 [SMILES: N#CCn1nc(Cl)c2c(NCC3CCN([S@](N)(=O)=NC#N)CC3)ccnc21] | (R)-4-(((3-chloro-1- (cyanomethyl)-1H- pyrazolo[3,4-b]pyridine-4- yl)amino)methyl)-N′- cyanopiperidine-1- sulfonimidamide | (400 MHz, DMSO-d6) δ ppm 8.15 (m, 1H), 8.00 (br s, 2H), 6.80 (m, 1H), 6.45 (m, 1H), 5.55 (s, 2H), 3.60- 3.70 (m, 2H), 3.20-3.40 (m, 2H), 2.60-2.75 (m, 2H), 1.70-1.90 (m, 3H), 1.20-1.40 (m, 2H) | 408.1 |
| 15G | 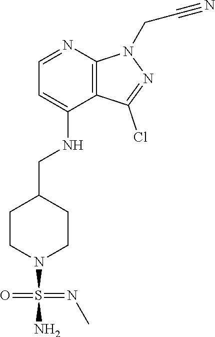 | (S)-4-(((3-chloro-1- (cyanomethyl)-1H- pyrazolo[3,4-b]pyridine-4- yl)amino)methyl)-N′- methylpiperidine-1- sulfonimidamide | (400 MHz, DMSO-d6) δ ppm 8.15 (m, 1H), 6.70- 6.80 (m, 1H), 6.30-6.40 (m, 1H), 5.55 (s, 2H), 3.10- 3.70 (m, 6H), 2.50 (s, 3H), 1.60-1.80 (m, 3H), 1.10- 1.30 (m, 2H) | 381.1 (MH⁺ − O) |
| 15H | 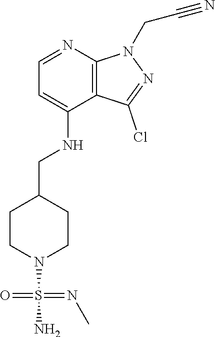 [SMILES: CN=[S@@](N)(=O)N1CCC(CNc2ccnc3c2c(Cl)nn3CC#N)CC1] | (R)-4-(((3-chloro-1- (cyanomethyl)-1H- pyrazolo[3,4-b]pyridine-4- yl)amino)methyl)-N′- methylpiperidine-1- sulfonimidamide | (400 MHz, DMSO-d6) δ ppm 8.15 (m, 1H), 6.70- 6.80 (m, 1H), 6.30-6.40 (m, 1H), 5.55 (s, 2H), 3.10- 3.70 (m, 6H), 2.50 (s, 3H), 1.60-1.80 (m, 3H), 1.10- 1.30 (m, 2H) | 397.1 |
| 15I | 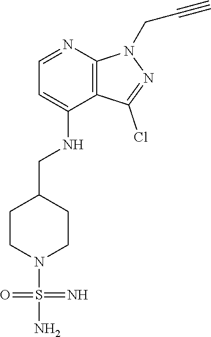 [SMILES: C#CCn1nc(Cl)c2c(NCC3CCN(S(=N)(N)=O)CC3)ccnc21] | 4-(((3-chloro-1-(prop-2-yn- 1-yl)-1H-pyrazolo[3,4- b]pyridine-4- yl)amino)methyl)piperidine- 1-sulfonimidamide | (400 MHz, DMSO-d6) δ ppm 8.90 (br s, 3H), 8.20 (m, 1H), 7.75 (m, 1H), 6.65 (m, 1H), 5.40 (s, 2H), 4.70- 4.75 (m, 2H), 3.40-3.50 (m, 3H), 2.80-2.90 (m, 2H), 1.80-1.90 (m, 3H), 1.30-1.40 (m, 2H) | 382.0 |

### Example 16

### Synthesis of N′-(cyclopropylmethyl)-4-((1-ethyl-1H-pyrazolo[3,4-b]pyridine-4-yl)oxy) benzenesulfonimidamide (Compound 16A)

N′-(cyclopropylmethyl)-4-((1-ethyl-1H-pyrazolo[3,4-b]pyridine-4-yl)oxy) benzenesulfonimidamide (Compound 16A), listed below, was prepared according to similar procedures as described herein (m/z means m/z (ES+)(M+)⁺).

TABLE 16

| Cmpd. | | | | |
|---|---|---|---|---|
| No. | Structure | Name | ¹H NMR | m/z |
| 16A | 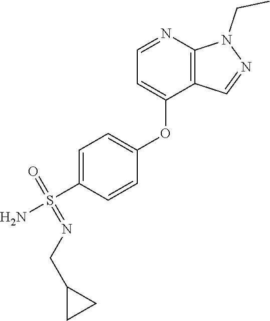 [SMILES: CCn1ncc2c(Oc3ccc(S(N)(=O)=NCC4CC4)cc3)ccnc21] | N′-(cyclopropylmethyl)-4- ((1-ethyl-1H-pyrazolo [3,4-b]pyridine-4-yl)oxy) benzenesulfonimidamide | (300 MHz, DMSO-d6) δ ppm 8.46 (d, J = 5.1 Hz, 1H), 8.03 (d, J = 8.7 Hz, 2H), 7.77 (s, 1H), 7.49 (d, J = 8.7 Hz, 2H), 6.63 (d, J = 5.4 Hz, 1H), 4.49 (q, J = 7.2 Hz, 2 H), 2.74 (d, J = 6.9 Hz, 2H), 1.42 (t, J = 7.2 Hz, 3H), 0.81-0.84 (m, 1H), 0.35- 0.37 (m, 2H), 0.10-0.12 (m, 2H). | 372.05 |

### Example 17

ENPP1 cGAMP Biochemical Assay

Compounds were added to 384 well Proxiplate (PerkinElmer, Waltham MA) at 100 nl/well from DMSO stocks using Echo (Labcyte, San Jose CA). Recombinant human ENPP1 protein (R&D Systems, Minneapolis MN) was prepared in assay buffer (50 mM Tris pH7.5, 1 mM TCEP pH7.0, 2 mM MgCl₂ and 0.005% Tween20) and added at 10 μl/well. The plate was sealed, centrifuged at 1000 rpm for 1 minute and incubated at room temperature for 30 minutes before 5 μl substrate 2′3′-cGAMP (R&D Systems, Minneapolis MN) in assay buffer was added to each well to a final concentration of 0.5 nM ENPP1 and 1000 nM 2′3′-cGAMP. The plate was sealed and centrifuged at 1000 rpm for 1 minute again. After incubation at 25° C. for 60 minutes, Terbium labelled AMP/GMP antibody and AMP/GMP HiLyte647 Tracer (BellBrook Labs, Fitchburg WI) in detection buffer (50 mM Tris pH7.5, 20 mM EDTA and 0.1% Prionex) were added to the reaction mixture to a final concentration of 2 nM (5 μl/well). The assay plate was incubated at room temperature for 120 minutes. TR-FRET signal was detected using Envision (PerkinElmer, Waltham MA).

ENPP3 cGAMP Biochemical Assay

Compounds were added to 384 well Proxiplate (PerkinElmer, Waltham MA) at 100 nl/well from DMSO stocks using Echo (Labcyte, San Jose CA). Recombinant human ENPP3 protein (Acro Biosystems, Newwark DE) was prepared in assay buffer (50 mM Tris pH7.5, 1 mM TCEP pH7.0, 2 mM MgCl₂, 2 mM CaCl₂), 2 mM ZnCl₂ and 0.005% Tween20) and added at 2 μl/well. The plate was sealed, centrifuged at 1000 rpm for 1 minute and incubated at room temperature for 30 minutes before 2 μl substrate 2′3′-cGAMP (R&D Systems, Minneapolis MN) in assay buffer was added to each well to a final concentration of 70.5 nM ENPP3 and 120 μM 2′3′-cGAMP. The plate was sealed and centrifuged at 1000 rpm for 1 minute again. After incubation at 25° C. for 60 minutes, Terbium labelled AMP/GMP antibody and AMP/GMP HiLyte647 Tracer (BellBrook Labs, Fitchburg WI) in detection buffer (50 mM Tris pH7.5, 20 mM EDTA and 0.1% Prionex) were added to the reaction mixture to a final concentration of 475 nM and 3 nM respectively. The assay plate was incubated at room temperature for 120 minutes. TR-FRET signal was detected using Envision (PerkinElmer, Waltham MA).

### Carbonic Anhydrase 1 Enzymatic Assay

Test compound and/or vehicle was preincubated with carbonic anhydrase I enzyme (15 μg/ml) isolated from human erythrocyte in reaction buffer (50 mM MOPS solution pH 7.5) for 15 minutes at 25° C. The reaction was initiated by addition of 500 μM 4-nitrophenyl acetate and incubated for 30 minutes. The plate was read on a spectrophotometer (Absorbance: 400 nm)

### Carbonic Anhydrase 2 Enzymatic Assay

Test compound and/or vehicle was preincubated with carbonic anhydrase II enzyme (15 μg/ml) isolated from human erythrocyte in reaction buffer (50 mM MOPS solution pH 7.5) for 15 minutes at 25° C. The reaction was initiated by addition of 500 μM 4-nitrophenyl acetate and incubated for 30 minutes. The plate was read on a spectrophotometer (Absorbance: 400 nm)

The activity of the compounds of the invention are presented in Table 17, wherein:
“A” denotes an IC₅₀ of less than 10 nM;
“B” denotes an IC₅₀ of from 10 nM to less than 100 nM;
“C” denotes an IC₅₀ of from 100 nM to less than 1000 nM; and
“D” denotes an IC₅₀ of 1000 nM or more.

TABLE 17
ENPP1, ENPP3 and Carbonic Anhydrase
I and II Activity (IC₅₀ (nM))

| Cmpd. | ENPP1 | ENPP3 | CA I | CA II |
|---|---|---|---|---|
| No. | IC₅₀ (nM) | IC₅₀ (nM) | IC₅₀ (nM) | IC₅₀ (nM) |
| 1A | B | C | C | D |
| 1B | B | B | C | C |
| 1C | B | D | | |
| 2A | B | D | C | D |
| 2B | B | C | B | D |
| 2C | C | C | | |
| 2D | B | D | | |
| 3A | C | D | D | D |
| 3B | A | C | | |
| 3C | A | C | | |
| 3D | A | C | | |
| 3E | A | D | | |
| 3F | C | D | | |
| 4A | B | D | D | D |
| 5A | B | C | | |
| 5B | B | D | | |
| 5C | B | C | | |
| 5D | A | C | | |
| 5E | A | C | | |
| 5F | A | C | | |
| 5G | B | D | | |
| 5H | A | C | | |
| 5I | A | D | | |
| 6A | B | D | | |
| 6B | A | B | | |
| 7A | C | D | | |
| 8A | A | C | | |
| 9A | A | B | | |
| 9B | A | B | | |
| 9C | A | D | | |
| 9D | A | B | | |
| 9E | D | D | | |
| 9F | A | B | | |
| 9G | C | D | | |
| 9H | A | B | | |
| 9I | A | C | | |
| 9J | A | C | | |
| 9K | A | C | | |
| 9L | A | C | | |
| 9M | B | D | | |
| 9N | A | D | | |
| 9O | A | C | | |
| 9P | A | B | | |
| 9Q | A | D | | |
| 9R | A | B | | |
| 9S | A | B | | |
| 9T | A | D | | |
| 10A | C | D | | |
| 10B | A | D | | |
| 10C | A | B | | |
| 10D | C | D | | |
| 10B | | | | |
| 10E | A | C | | |
| 10F | B | D | | |
| 11A | A | B | | |
| 11B | A | C | | |
| 11C | A | D | | |
| 12A | A | B | | |
| 12B | A | C | | |
| 12C | B | D | | |
| 12D | B | D | | |
| 12E | B | D | | |
| 12F | A | B | | |
| 12G | A | C | D | D |
| 12H | A | A | C | D |
| 12I | A | A | C | D |
| 12J | A | A | C | D |
| 12K | A | A | C | C |
| 13A | A | B | | |
| 13B | A | B | | |
| 13C | A | D | | |
| 13D | A | B | | |
| 13C | | | | |
| 13E | A | B | | |
| 13D | | | | |
| 13F | C | D | | |
| 13G | B | D | | |
| 13E | | | | |
| 13H | B | D | | |
| 14A | A | D | | |
| 14B | B | D | | |
| 14C | B | D | | |
| 14D | B | D | | |
| 14E | B | D | D | D |
| 14F | A | C | | |
| 14G | D | D | | |
| 14H | A | D | | |
| 14I | A | D | | |
| 14J | C | D | | |
| 14K | A | D | | |
| 15A | A | B | | |
| 15B | A | A | | |
| 15C | A | D | | |
| 15D | A | B | D | D |
| 15E | A | A | C | D |
| 15F | A | A | C | C |
| 15G | A | C | D | D |
| 15H | A | C | D | D |
| 15I | A | C | | |
| 16A | D | D | | |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by disclosure.

This application claims the benefit of priority to U.S. Provisional Application No. 63/341,991, filed May 13, 2022, which application is hereby incorporated by reference in its entirety.

1. A compound having the structure of Formula (I): 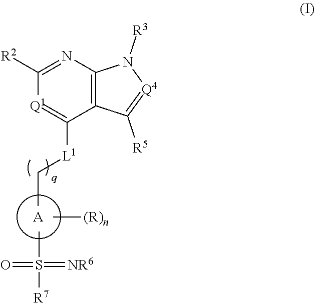
or a pharmaceutically acceptable salt thereof, wherein
Q¹ is CR¹;
Q⁴ is N;
L¹ is —NR^{A}—
A is phenyl or piperidinyl;
R¹ is H;
R² is H;
R³ is —C₁₋₄alkyl-R^{3a}, C₁₋₄haloalkyl-R^{3a}, C₂₋₄alkenyl, or —C₂₋₄alkynyl;
R^{3a} is H, halo, or —CN;
R⁵ is H, halo, C₁₋₄alkyl, or C₁₋₄haloalkyl;
R⁶ is H, or C₁₋₄alkyl;
R⁷ is —C₁₋₄alkyl-R^{7c};
R^{7c} is H, —CN, or halo;
R is, at each occurrence, independently —CN, halo, C₁₋₄alkyl, or C₁₋₄haloalkyl;
R^{A} is H, C₁₋₄alkyl, or C₁₋₄haloalkyl;
n is 0, 1, 2, or 3; and
q is 1.

2-5. (canceled)

6. The compound of claim 1, having the structure of Formula (II): 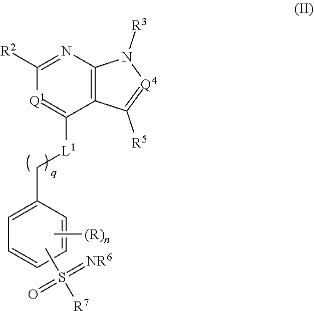
or a pharmaceutically acceptable salt thereof, wherein
R^{3a} is H, or —CN.

7-15. (canceled)

16. The compound of claim 6, having the structure of any one of Formula (V-A), Formula (V-B), or Formula (V-C): 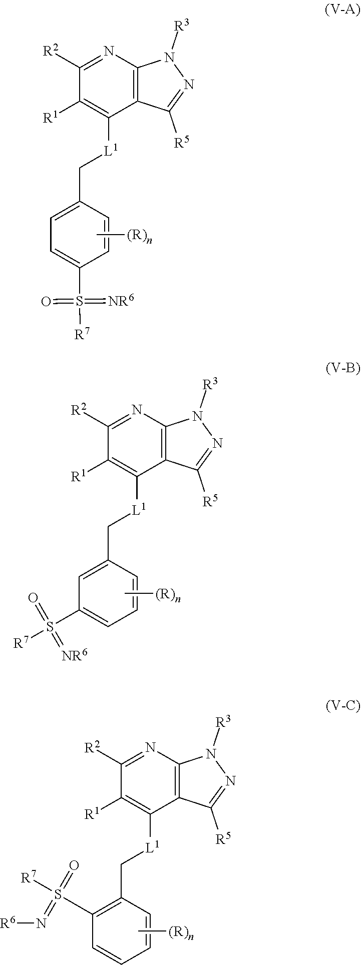
or a pharmaceutically acceptable salt thereof.

17-23. (canceled)

24. The compound of claim 1, having the structure of Formula (VII): 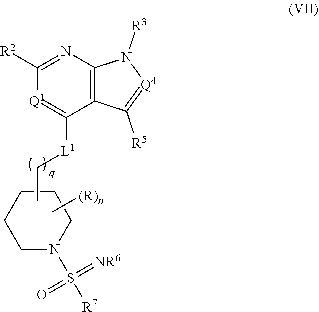
or a pharmaceutically acceptable salt thereof, wherein
R^{3a} is H or —CN.

25-31. (canceled)

32. The compound of claim 24, having the structure of any one of Formula (X-A), Formula (X-B), or Formula (X-C), 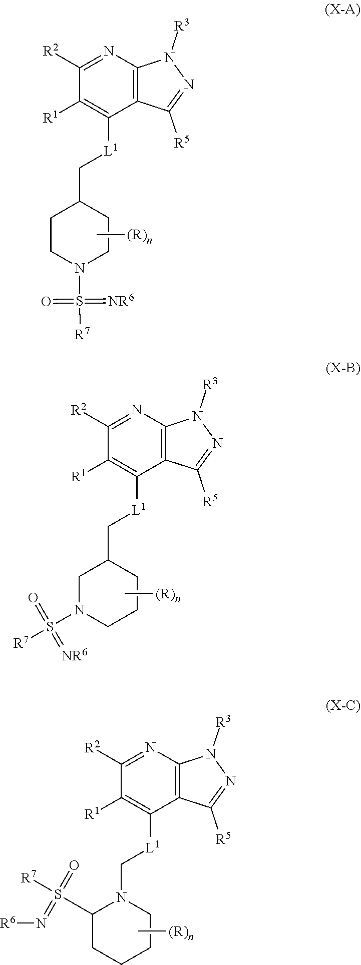
or a pharmaceutically acceptable salt thereof.

33-58. (canceled)

59. The compound of claim 1, wherein R³ is:

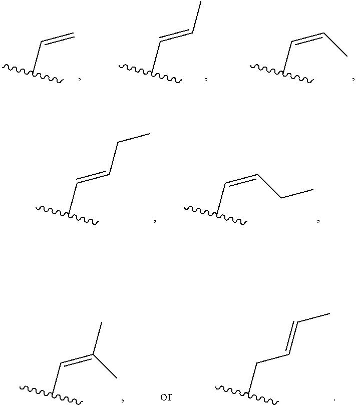

60. (canceled)

61. The compound of claim 1, wherein R³ is:

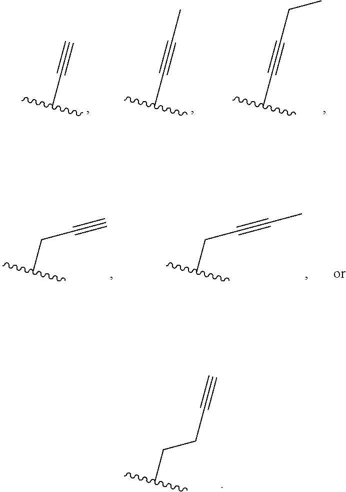

62. The compound of claim 61, wherein R³ is:

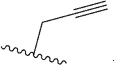 [SMILES: *CC#C]

63-80. (canceled)

81. The compound of claim 1, wherein R^{7c} is H.

82. The compound of claim 1, wherein R⁷ is methyl or ethyl.

83. The compound of claim 1, salt wherein R^{7c} is —CN.

84. The compound of claim 1, wherein R⁷ is —CH₂CN or —CH₂CH₂CN.

85-90. (canceled)

91. A compound having the structure of any one of the compounds of Table 1, or a pharmaceutically acceptable salt thereof.

92. A pharmaceutical composition comprising a compound of claim 1, or a pharmaceutically acceptable salt thereof; and a pharmaceutically acceptable carrier, diluent, or excipient.

93. (canceled)

94. A method for inhibiting the activity of ENNP1 comprising contacting ENPP1 with a compound of claim 1, or a pharmaceutically acceptable salt thereof.

95. A method of treating a subject having a disease or disorder associated with the activity of ENPP1 comprising administering to the subject in need thereof a therapeutically effective amount of a compound of claim 1, or a pharmaceutically acceptable salt thereof.

96. The method of claim 95, wherein the disease or disorder associated with the activity of ENPP1 is an infection, a proliferative disorder, cardiovascular disease, diabetes, insulin resistance, chondrocalcinosis, calcium pyrophosphate deposition disorder, pseudogout, or hypophosphatasia.

97. A method of treating a subject having a proliferative disorder comprising administering to the subject in need thereof a therapeutically effective amount of a compound of claim 1, or a pharmaceutically acceptable salt thereof.

98. The method of claim 97, wherein the proliferative disorder is cancer.

99. The method of claim 98, wherein the cancer is a solid tumor.

100. The method of claim 99, wherein the solid tumor cancer is adrenal cancer, liver cancer, kidney cancer, bladder cancer, breast cancer, colon cancer, gastric cancer, ovarian cancer, cervical cancer, uterine cancer, esophageal cancer, colorectal cancer, prostate cancer, pancreatic cancer, small cell lung cancer, non-small cell lung cancer, thyroid cancer, carcinomas, sarcomas, glioblastomas, melanoma, a head and neck tumor.

101-144. (canceled)
